(19) United States
(12) Patent Application Publication
(10) Pub. No.: US 2003/0153905 A1
(43) Pub. Date: Aug. 14, 2003

(54) Selective ablation system

(76) Inventors: Stuart Denzil Edwards, Corral De Tierra, CA (US); Thom Wehman, Cupertino, CA (US); Ted Kucklick, Los Gatos, CA (US); Peter H. Muller, Los Gatos, CA (US); Bruno Strul, Portola Valley, CA (US); Brian Wong, Irvine, CA (US)

Correspondence Address: GLENN PATENT GROUP, 3475 EDISON WAY, SUITE L, MENLO PARK, CA, 94025, US

(21) Appl. No.: 10/059,098

(22) Filed: Jan. 25, 2002

(51) Int. Cl. A61B 18/18

(52) U.S. Cl. 606/41

(57) ABSTRACT

Structures, processes, and mechanisms are provided for the ablation of hollow organs. Ablation structures, having deployable electrically conductive probes, are placed within a hollow organ, such as a stomach. The ablation structure typically includes a distension mechanism, whereby the hollow organ is controllably distended. The electrically conductive probes are then deployed, such that the probes extend make electrical contact with the tissue of the hollow organ, typically by extending through a mucosal layer of the hollow organ. The electrically conductive probes are typically deployed by extension of movable electrically conductive probes, from a first protected position to a second extended position. In alternate embodiments of the ablation system, the ablation apparatus includes means for vacuum-directed contact between the tissue and the electrically conductive probes. When, the electrically conductive probes are deployed to make electrical contact with the tissue of the hollow organ, the probes are typically used for monopolar or bipolar ablation, including mapping and/or ablation (zapping).

### FIELD OF THE INVENTION

The invention relates to the field of ablation systems. More particularly, the invention relates to the measurement of impedance and the application of energy for hollow organ ablation applications and systems.

### BACKGROUND OF THE INVENTION

Obesity is directly associated with disorders such as osteoarthritus (especially in the hips), sciatica, varicose veins, thromboembolism, ventral and hiatal hernias, hypertension, insulin resistance, and hyperinsulinemia.

All these conditions can be ameliorated by treatment of obesity, providing the weight loss is significant and enduring.

The known art of treating obesity includes behavioral strategies, various different pharmaceutical interventions and surgery.

One problem in the known art of behavioral strategies is patient compliance. Extremely high levels of patient compliance over a long period of time are required to produce significant weight loss.

Problems in the known art of pharmaceutical intervention include drug dependence and side effects. Treatment with amphetamine analogs requires habitual use of an addictive drug to produce a significant weight loss. Treatment with drugs such as dexfenfluramine and fenfluramine is frequently associated with primary pulmonary hypertension and cardiac valve abnormalities. Drugs such as sibutramine cause a substantial increase in blood pressure in a large number of patients.

The known art of surgical treatment of obesity includes operative procedures such as end-to-end anastomosis of about 38 cm of proximal jejunum to 10 cm of terminal ileum and other variants of jejunoileal manipulation. While such procedures are extremely effective, the overall rates of surgical mortality and associated hepatic dysfunction are so high that this treatment is only indicated for younger patients who are morbidly obese.

It would be advantageous to provide a structure and process, whereby the acquisition of data, such as impedance, voltage, current, biological nerve signals, and/or temperature can readily be performed on a hollow organ with a series of electrodes or deployable probes. The development of such a measurement system would constitute a major technological advance.

It would also be advantageous to provide a ablation structure and process, whereby ablation can readily be performed on a hollow organ with a series of electrodes or deployable probes, such as for the ablation of diseased tissues or to increase the relative muscle tone of sphincters. The development of such a measurement system would constitute a major technological advance. The development of such an ablation system would constitute a further technological advance.

Furthermore, it would be advantageous to provide a method and system for the treatment of obesity, such as to create a sense of satiety in a patient, that produces reasonably rapid weight loss, long term results, low surgical mortality, and few side effects, which can be performed under local anesthesia. The development of such a system would constitute a further technological advance.

### SUMMARY OF THE INVENTION

Systems are provided for the ablation of hollow organs. An ablation structure, having deployable electrically conductive probes, is placed within a hollow organ, such as a stomach. The ablation structure typically includes a distension mechanism, whereby the hollow organ is controllably distended. The electrically conductive probes are then deployed, such that the probes make electrical contact with the tissue of the hollow organ, typically by extending through a mycosal layer of the hollow organ. The electrically conductive probes are typically deployed by an extension of movable electrically conductive probes, from a first protected position to a second extended position. In alternate embodiments of the ablation system, the ablation apparatus includes means for vacuum-directed contact between the tissue and the electrically conductive probes. When the electrically conductive probes are deployed to make electrical contact with the tissue of the hollow organ, the probes are preferably used for the procurement of mapping data, as well as for the application of ablation energy. The ablation system also preferably comprises one or more thermal sensors in thermal contact with the electrically conductive probes.

### BRIEF DESCRIPTION OF THE DRAWINGS

FIG. 1 is simplified diagram of a compliant ablation system;

FIG. 2 is a first perspective view of an expandable ablation apparatus having deployable needles;

FIG. 3 is a perspective view of a hand piece attached to an expandable ablation apparatus having deployable needles;

FIG. 4 is a side perspective view of an expandable ablation apparatus having deployable needles;

FIG. 5 is a partial detailed perspective view of deployable needles for an expandable ablation apparatus;

FIG. 6 is a partial cross sectional view of a deployable needle for an expandable ablation apparatus;

FIG. 7 is a first partial perspective view of an expandable ablation apparatus having a poppet needle array in a protected position;

FIG. 8 is a second partial perspective view of an expandable ablation apparatus having a poppet needle array in an extended position;

FIG. 9 is a partial cutaway view of an expandable ablation apparatus located within a hollow organ;

FIG. 10 is a partial cross sectional view of a poppet needle in a protected position in relation to tissue;

FIG. 11 is a partial cross sectional view of a poppet needle in an extended position in relation to tissue;

FIG. 12 is a partial cross sectional view of a self-sheathing needle and balloon system;

FIG. 13 is a partial cutaway perspective view of a self-sheathing needle and balloon system;

FIG. 14 is a perspective view of a self-sheathing needle and balloon system in an expended position;

FIG. 15 is a detailed cross sectional view of an ablation needle having vacuum actuation for tissue contact;

FIG. 16 is a detailed partial cross sectional view of an ablation structure having a vacuum ablation needle, without vacuum activation;

FIG. 17 is a detailed partial cross sectional view of an ablation structure having a vacuum ablation needle, with vacuum activation;

FIG. 18 is a detailed partial cross sectional view of an ablation structure having a hydraulic piston ablation needle, without hydraulic activation;

FIG. 19 is a detailed partial cross sectional view of an ablation structure having a hydraulic piston ablation needle, with hydraulic activation;

FIG. 20 is a perspective view of a balloon ablation structure having a deployable piston needle array;

FIG. 21 is a perspective view of a basket ablation structure having a deployable piston needle array;

FIG. 22 is a partial cross sectional view of an ablation structure having a distending structure, before needle deployment;

FIG. 23 is a partial cross sectional view of an ablation structure having a distending structure, after needle deployment;

FIG. 24 is a perspective view of an ablation structure having an expandable distension balloon structure, before needle deployment;

FIG. 25 is a functional view of an ablation structure having an expandable distension balloon structure and an integrated advancement and retrieval mechanism;

FIG. 26 is a partial cross sectional view of a balloon structure having a deployable needle and conductive solution ports;

FIG. 27 is a functional side view of internal electrical connections for an ablation system having extendable electrodes;

FIG. 28 is a flow diagram of first embodiment of a staged balloon ablation process;

FIG. 29 shows the insertion of a gastro tube in a first embodiment of a staged balloon ablation process;

FIG. 30 is a detailed perspective view of an expandable funnel end of a gastro tube;

FIG. 31 shows the expansion of the funnel end of a gastro tube in a first embodiment of a staged balloon ablation process;

FIG. 32 is a detailed perspective view of an expanded funnel end of a gastro tube;

FIG. 33 shows the insertion of a staged balloon assembly though a gastro tube in the first embodiment of a staged balloon ablation process;

FIG. 34 shows inflation of a first outer balloon and stomach distension in the first embodiment of a staged balloon ablation process;

FIG. 35 shows inflation of a probe needle balloon in the first embodiment of a staged balloon ablation process;

FIG. 36 is a detail view of inflation of a probe needle balloon in the first embodiment of a staged balloon ablation process;

FIG. 37 shows inflation of an inner probe needle deployment balloon in the first embodiment of a staged balloon ablation process;

FIG. 38 is a detail view of needle deployment in the first embodiment of a staged balloon ablation process;

FIG. 39 shows selective ablation through deployed needles in the first embodiment of a staged balloon ablation process;

FIG. 40 is a detail view of selective ablation through a deployed needle in the first embodiment of a staged balloon ablation process;

FIG. 41 shows deflation of the inner probe needle deployment balloon and the probe needle balloon in the first embodiment of a staged balloon ablation process;

FIG. 42 shows the removal of the deflated inner probe needle deployment balloon and the probe needle balloon in the first embodiment of a staged balloon ablation process;

FIG. 43 shows the deflation of a first outer balloon in the first embodiment of a staged balloon ablation process;

FIG. 44 shows the removal of the deflated first outer balloon in the first embodiment of a staged balloon ablation process;

FIG. 45 shows funnel-end retraction for the gastro tube in the first embodiment of a staged balloon ablation process;

FIG. 46 shows the removal of the gastro tube in the first embodiment of a staged balloon ablation process;

FIG. 47 is a flow diagram of second embodiment of a staged balloon ablation process;

FIG. 48 shows the insertion of a gastro tube in a second embodiment of a staged balloon ablation process;

FIG. 49 is a detailed perspective view of an expandable funnel end of a gastro tube;

FIG. 50 shows the expansion of the funnel end of a gastro tube in a second embodiment of a staged balloon ablation process;

FIG. 51 is a detailed perspective view of an expanded funnel end of a gastro tube;

FIG. 52 shows the insertion of a staged balloon assembly though a gastro tube in the second embodiment of a staged balloon ablation process;

FIG. 53 shows inflation of a first outer balloon and stomach distension in the second embodiment of a staged balloon ablation process;

FIG. 54 shows the introduction of saline solution into the first outer balloon in the second embodiment of a staged balloon ablation process;

FIG. 55 shows inflation of a probe needle balloon in the second embodiment of a staged balloon ablation process;

FIG. 56 is a detail view of inflation of a probe needle balloon in the second embodiment of a staged balloon ablation process;

FIG. 57 shows inflation of an inner probe needle deployment balloon in the second embodiment of a staged balloon ablation process;

FIG. 58 is a detail view of needle deployment in the second embodiment of a staged balloon ablation process;

FIG. 59 shows selective ablation through deployed needles in the second embodiment of a staged balloon ablation process;

FIG. 60 is a detail view of selective ablation through a deployed needle in the second embodiment of a staged balloon ablation process;

FIG. 61 shows deflation of the inner probe needle deployment balloon and the probe needle balloon in the second embodiment of a staged balloon ablation process;

FIG. 62 shows the removal of the deflated inner probe needle deployment balloon and the probe needle balloon in the second embodiment of a staged balloon ablation process;

FIG. 63 shows the deflation of the outer balloon and the removal of saline solution in the second embodiment of a staged balloon ablation process;

FIG. 64 shows the removal of the deflated first outer balloon in the second embodiment of a staged balloon ablation process;

FIG. 65 shows funnel-end retraction and removal for the gastro tube in the second embodiment of a staged balloon ablation process;

FIG. 66 is a partial perspective view of bi-polar surface connections for an ablation balloon;

FIG. 67 is a partial plan view of conductive traces on a polymer substrate;

FIG. 68 is a detailed partial perspective view of overlapping conductive traces and an ablation zone;

FIG. 69 is a partial perspective view of an ablation balloon having overlaid bi-polar surface connections located within a stomach;

FIG. 70 is a schematic plan view of an alternate embodiment for bi-polar surface conductors;

FIG. 71 is a detailed schematic plan view of bi-polar surface conductors having coolant ports with a defined ablation zone;

FIG. 72 is a perspective assembly view of an alternate ablation apparatus having vacuum deployment;

FIG. 73 is a partial cross sectional view of an alternate ablation apparatus having vacuum probe needle deployment;

FIG. 74 is a detailed partial cross sectional view of vacuum probe needle deployment;

FIG. 75 is a perspective view of an octopus basket arm ablation apparatus;

FIG. 76 is a perspective view of a balloon arm ablation;

FIG. 77 is a detail view of vacuum needle deployment for an ablation apparatus;

FIG. 78 is a perspective view of an inflatable bladder needle driver ablation apparatus;

FIG. 79 is a partial perspective cutaway view of an inflatable bladder in a first undeployed position;

FIG. 80 is a partial perspective cutaway view of an inflatable bladder in a second deployed position;

FIG. 81 is a partial perspective view of inflatable bladder needle driver ablation apparatus located within a stomach, and further comprising a distending balloon;

FIG. 82 is a perspective view of an RF needle tack strip and a protective sleeve;

FIG. 83 is a partial cross sectional view of an RF needle tack strip having an inflatable bladder in a first undeployed position with a channel;

FIG. 84 is a partial cross sectional view of an RF needle tack strip having an inflatable bladder in a second deployed position with a channel;

FIG. 85 is a perspective view of an RF needle tack strip having a flex circuit and an etched thermocouple array;

FIG. 86 is a partial cross sectional view of an RF needle tack strip having a flex circuit and an etched thermocouple array;

FIG. 87 is a perspective assembly view of a needle driver apparatus having externally-mounted tack strip probes;

FIG. 88 is a perspective assembly view of a mandrel needle driver apparatus having tack strip probes;

FIG. 89 is a perspective view of a mandrel needle driver apparatus having tack strip probes;

FIG. 90 is a partial cross sectional view of an RF needle tack strip having an inflatable driver in a first undeployed position within a channel;

FIG. 91 is a partial cross sectional view of an RF needle tack strip having an inflatable driver in a second deployed position within and extending from a channel;

FIG. 92 is a partial cross sectional view of a hypotube ablation needle;

FIG. 93 is a perspective view of a hypotube tack strip;

FIG. 94 is a perspective view of a center punch-up tack strip;

FIG. 95 is a perspective view of a side punch-up tack strip;

FIG. 96 is a perspective view of a spot welded hypotube tack strip;

FIG. 97 is a perspective view of a spot welded flat needle tack strip;

FIG. 98 is a partial cutaway view of an ablation region established within the tissue of a hollow organ;

FIG. 99 is a perspective view of a formed needle probe;

FIG. 100 is a perspective view of an integrated spring needle probe;

FIG. 101 is a partial cutaway view of an integrated spring needle probe located between an inner activation balloon and an outer distension balloon;

FIG. 102 is a partial perspective view of an integrated spring needle probe;

FIG. 103 is a partial perspective view of an alternate integrated spring needle probe;

FIG. 104 is a partial cutaway view of a leaf spring needle probe in an undeployed position;

FIG. 105 is a partial cutaway view of a leaf spring needle probe in a deployed position;

FIG. 106 is a partial cutaway view of an elastomer spring needle probe in an undeployed position;

FIG. 107 is a partial cutaway view of an elastomer needle probe in a deployed position;

FIG. 108 is a partial cutaway view of a coil spring needle probe in an undeployed position;

FIG. 109 is a partial cutaway view of a coil spring needle probe in a deployed position;

FIG. 110 is a simplified functional block diagram of the deployable ablation system;

FIG. 111 is a partial cutaway view of an expandable ablation device within a pleated hollow organ;

FIG. 112 is a partial cutaway view of a partially expanded ablation device within a distended pleated hollow organ;

FIG. 113 is a partial cutaway view of an ablation substantially across a meridian region within a distended pleated hollow organ;

FIG. 114 is a partial cutaway view of selective ablation over a portion of a distended pleated hollow organ;

FIG. 115 is a partial cutaway view showing deflation and rotation of a compliant ablation device within pleated hollow organ;

FIG. 116 is a partial cutaway view of selective ablation over a portion of a distended pleated hollow organ from a repositioned compliant ablation device;

FIG. 117 is a functional block diagram showing bipolar ablation within a hollow organ;

FIG. 118 is a functional block diagram showing monopolar ablation within a hollow organ;

FIG. 119 is a side view of a compliant probe balloon having longitudinal probe groups;

FIG. 120 is a side view of a compliant probe balloon having latitudinal probe groups;

FIG. 121 is a side view of a compliant probe balloon having longitudinal quadrant probe groups; and

FIG. 122 is a side view of a compliant probe balloon having latitudinal quadrant probe groups.

### DETAILED DESCRIPTION OF PREFERRED EMBODIMENTS

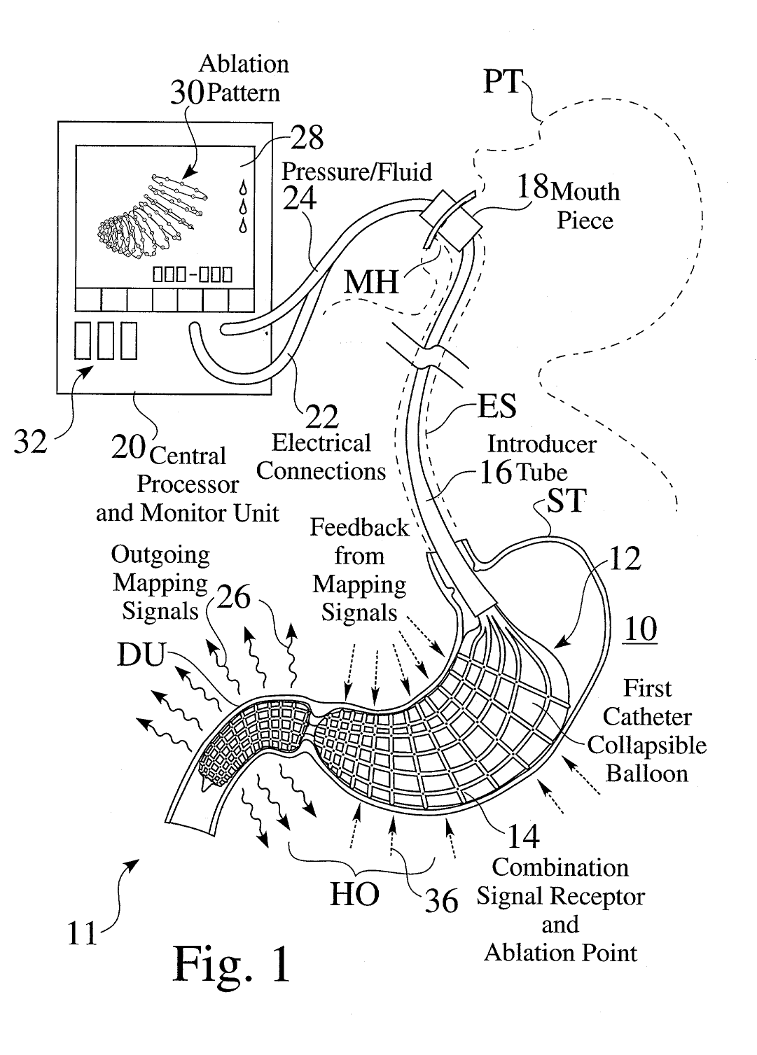

FIG. 1 is simplified diagram of a compliant ablation system 11. A deployable ablation apparatus 10, comprising a compliant balloon structure 12, is located within a hollow organ HO. In FIG. 1, the exemplary hollow organ is shown as a stomach ST, extending into a duodenum DU. The compliant balloon 12 comprises one or more deployable electrically conductive probes 14, i.e. needles 14, which controllably come into contact with the tissue TI of the hollow organ HO. It will be appreciated by those skilled in the art that such probe may comprise any active element, e.g. a source of radiation such as an RF or microwave emitter or a laser.

The compliant balloon structure 12 is typically inserted into the hollow organ HO, such as through a hollow introducer tube 16. For the compliant ablation system 10 shown in FIG. 1, the introducer tube 16 further comprises a mouthpiece 18, whereby the introducer tube 16 can readily be inserted into the mouth MH and through the esophagus ES of a patient PT.

The ablation apparatus 10 is typically connected to an external processor and monitor unit 20, having electrical connections 22. In some embodiments, one or more pressure and/or fluid connections 24 are also provided, such as to provide distension of the hollow organ HO, or to provide deployment of the electrically conductive probes 14 into the tissue TI of the hollow organ HO.

In FIG. 1, the electrical connections 22 provide mapping signals 26, such as but not limited to impedance, current, voltage, temperature, or biological nerve signals. The external processor and monitor unit 20 preferably comprises a display 28, whereby mapping signals or control parameters, such as an ablation map 30 can be displayed, based upon the mapping signal data 26. The external processor and monitor unit 20 also preferably comprises user controls 32, such as but not limited to the control of pressure or fluid to distend the hollow organ HO, the deployment of the electrically conductive probes 14, the acquisition of mapping signal data 26, and/or the application of energy through one or more of the electrically conductive probes 14, for ablation 36 of at least a portion of the tissue TI of the hollow organ HO.

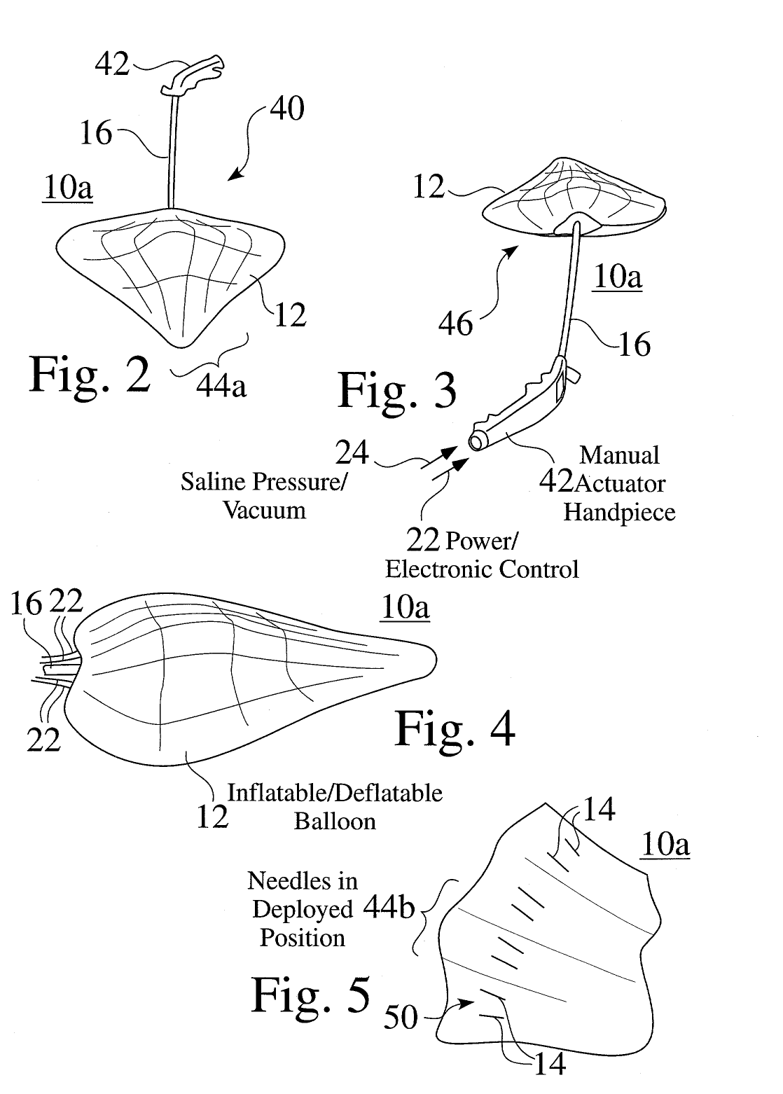

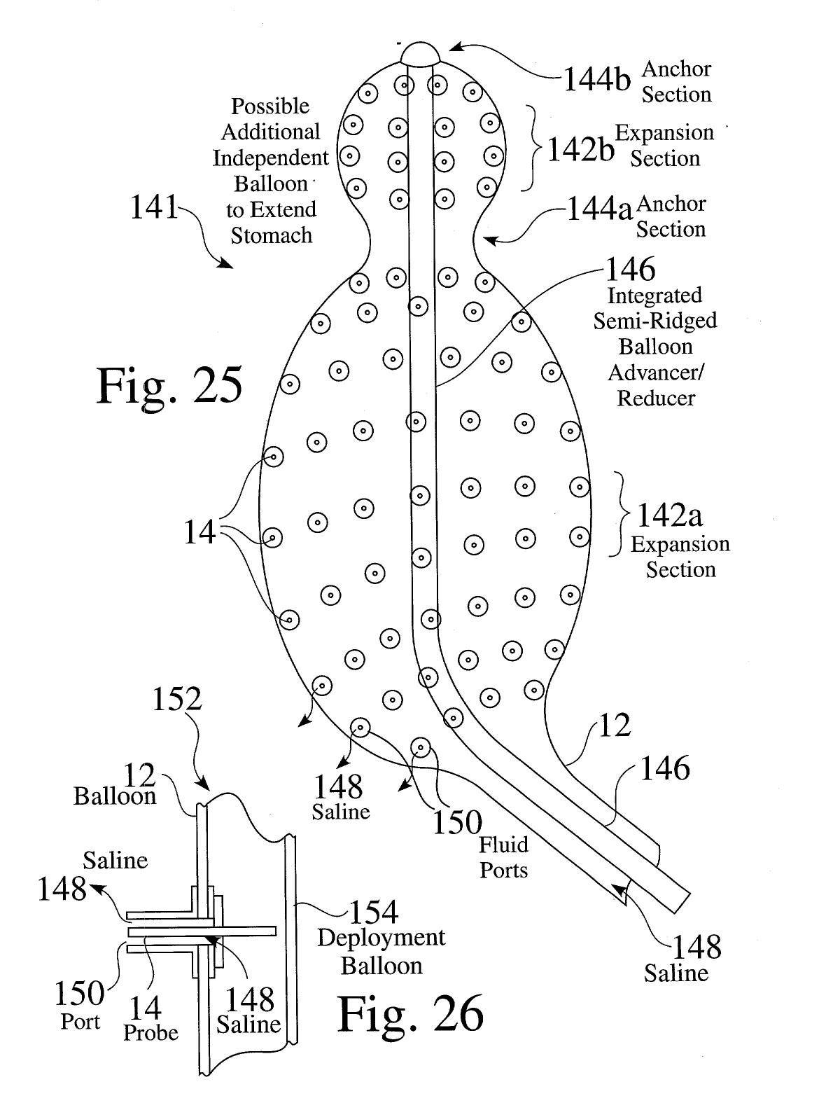

FIG. 2 is a first perspective view 40 of an expandable ablation apparatus 10a having a handpiece 42 connected to the introducer tube 16. FIG. 3 is a perspective view 46 of a handpiece 42 for a expandable ablation apparatus 10a having deployable needles 14. The compliant balloon structure 12 includes deployable needles 14 (FIG. 5), which are substantially protected in a first undeployed position 44a, such that the tips 50 (FIG. 5) of the electrically conductive probes 14 do not make contact with a hollow organ HO during installation or removal procedures. As seen in FIG. 3, the handpiece 44 provides modular connectivity for external devices, such as for electrical connections 22 and pressure or vacuum connections 24. The handpiece 44 may similarly include connections for other sensors, such as for temperature sensors 458 (FIG. 85), or for process fluid connections, such as for saline 148 (FIG. 25, FIG. 26). FIG. 4 is a side perspective view of an expandable ablation apparatus 10a having deployable needles 14. FIG. 5 is a partial detailed perspective view of deployable needles 14 for an expandable ablation apparatus 10a, wherein needles 14 are extended in a second deployed position 44b, such that the tips 50 of the electrically conductive probe needles 14 can make contact with the tissue TI of a hollow organ HO, such as to provide mapping signals 26, and/or to provide ablation energy signals 36.

FIG. 6 is a partial cross sectional schematic view 52 of a deployable electrically conductive probe needle 14 for an expandable ablation apparatus 10. The electrically conductive probe needle 14 is mounted to a substrate 54, such as the body of a compliant balloon 12. One or more electrical connections 56 are provided to each of the electrically conductive probe needles 14, such as though wires, traces, or though an electrically conductive saline solution 148 (FIG. 25, FIG. 26), such as through a fluid conduit 58, or even directly through the interior 60 of the ablation apparatus 10, as seen in FIG. 8. The electrical connections 56 shown in FIG. 6 are used for impedance data 26, temperature data, and/or for applied energy 26.

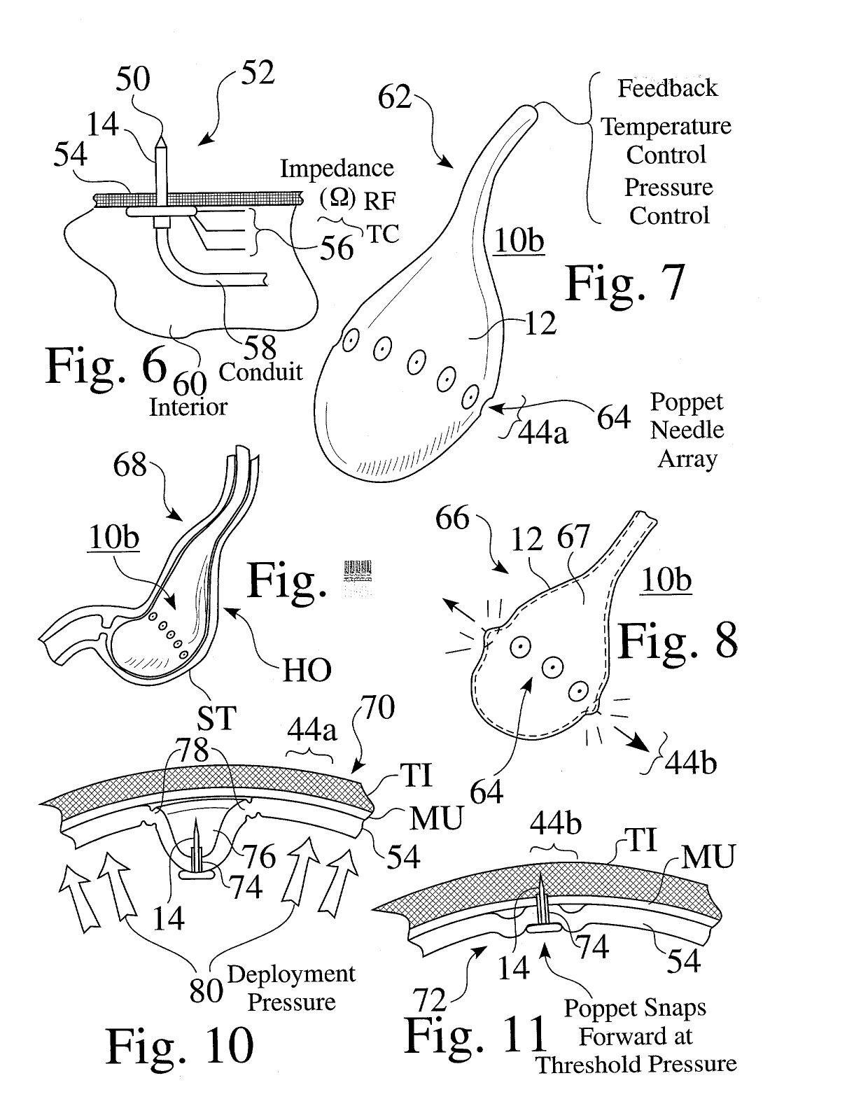

FIG. 7 is a first partial perspective view 62 of an expandable ablation apparatus 10b having a poppet needle array 64 of electrically conductive probe needles 14 in an undeployed, i.e. protected position 44a, in which the tips 50 of the probe needles 14 are protected from making contact with a hollow organ HO, such that the ablation apparatus 10b may readily be placed, positioned, or removed. FIG. 8 is a second partial perspective view 66 of an expandable ablation apparatus 10b having a poppet needle array 64 in an extended position 44b. While the poppet needle array 64 shown in FIG. 7 and FIG. 8 has a ring configuration, the poppet needle array 64 can preferably be located anywhere on the surface of the expandable ablation apparatus 10b, and can substantially cover all or only a portion of the surface of the expandable ablation apparatus 10b.

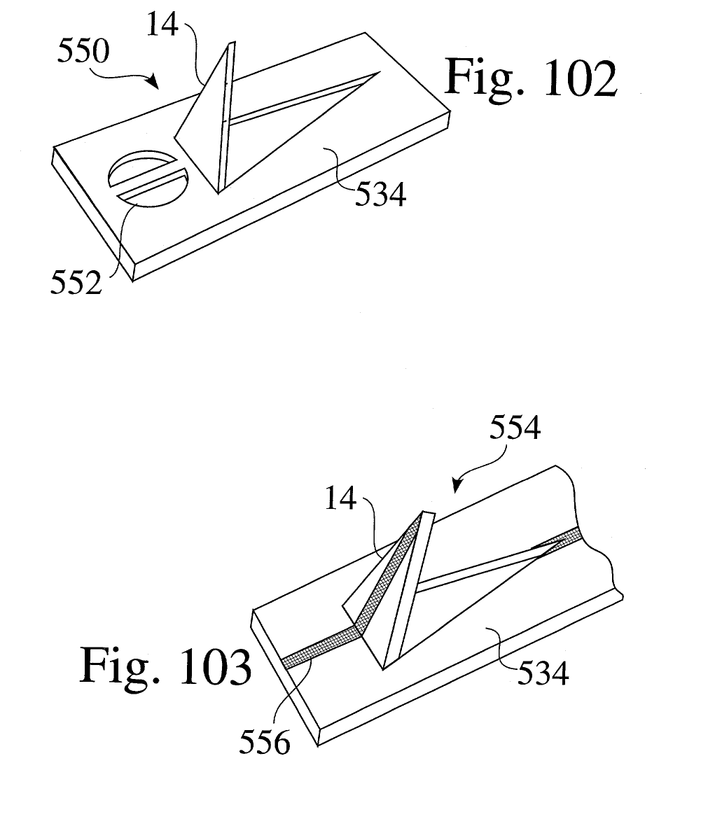

FIG. 9 is a partial cutaway view 68 of an expandable ablation apparatus 10b located within a hollow organ HO, such as a stomach ST. When the expandable ablation apparatus 10b is not distended 102 (FIG. 102) and is undeployed, 44a, the apparatus can easily be placed, positioned, or removed in relation to a hollow organ HO, as the tips 50 of the electrically conductive probe needles 14 do not make contact with the hollow organ HO.

FIG. 10 is a partial cross sectional view 70 of a poppet needle 14 in a protected position 44a in relation to tissue TI. FIG. 11 is a partial cross sectional view 72 of a poppet needle 14 in an extended position 44b in relation to tissue TI. The internal surface of a hollow organ HO typically includes a mucosal layer MU. The poppet needles 14 preferably include an electrically insulative region 74, which substantially insulates the mucosal layer MU from direct electrical contact with the needles 14. The insulative region 14 is preferably comprised of an inert polymer, such as nylon, or a fluoropolymer, such as PET.

For an ablation apparatus 10b having a poppet needle array 64, the substrate 54 typically includes recess regions 76 surrounding the needles 14, such that the needles 14 are located below the external surface of the apparatus 10b when the apparatus is in an undeployed position 44a. The recess region 76 shown in FIG. 11 further comprises an extension detail 78, such as a region having a ribbed cross section i.e. similar to a flexible ribbed region of an acoustic speaker, and/or a reduced substrate thickness, to promote movement of the recessed region 76 from the undeployed position 44a to the deployed position 44b, when the compliant balloon 12 is acted upon by a deployment pressure 80, such as provided by a pneumatic or hydraulic source 116 (FIG. 19). In FIG. 10, the deployment pressure 80 is provided directly to the interior 60 of the apparatus 10, wherein the deployment pressure 80 is greater than a distension pressure 102 (FIG. 17) that is applied to the interior 60 of the apparatus 10. In some embodiments of the ablation apparatus 10, the deployment pressure 80 is applied at a generally rapid rate, to promote movement of the needle probes 14 into the tissue TI, and to prevent localized “tenting”, i.e. deflection, the tissue TI.

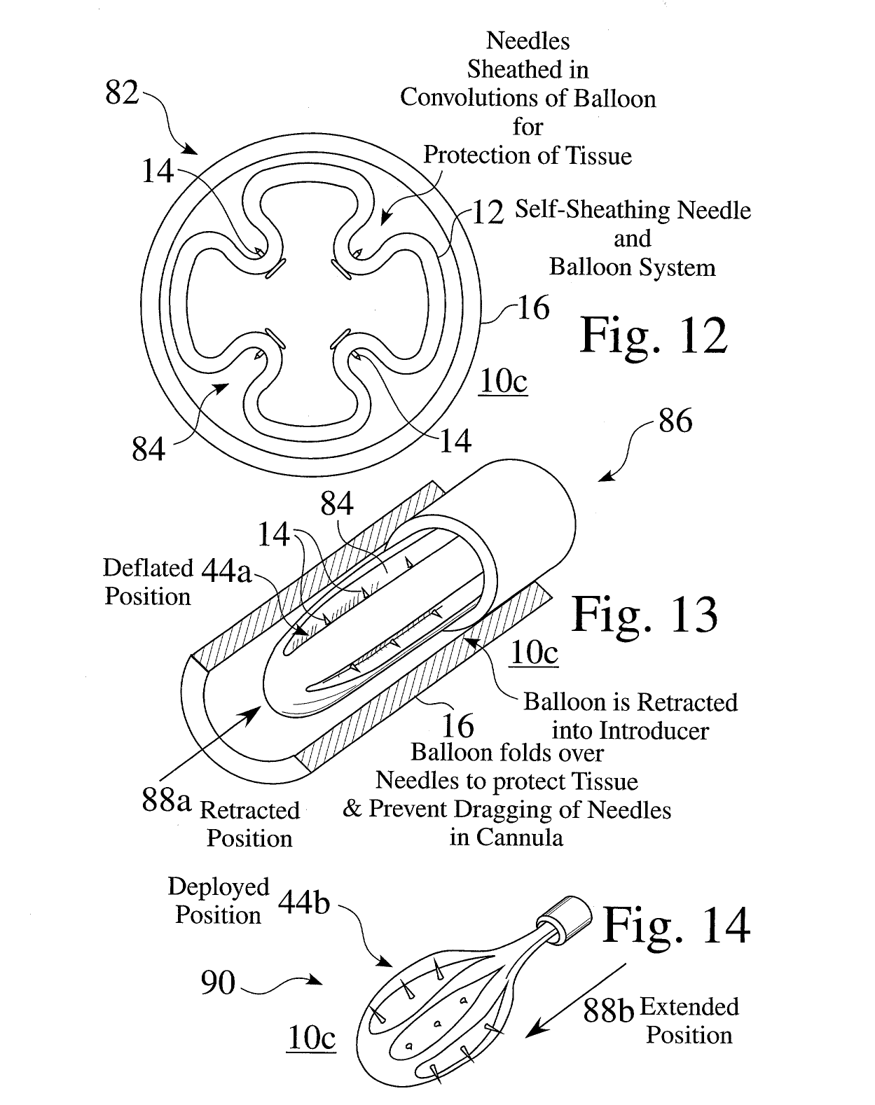

FIG. 12 is a partial cross sectional view 82 of a self-sheathing needle and balloon system 10c, in which the compliant balloon structure 12 has one or more convoluted recessed areas 84, such that the balloon 12 can be retracted within an introducer 16, and can be extended from the introducer 16, within a hollow organ HO. One or more electrically conductive probes 14 are located within each convolution 84. FIG. 13 is a partial cutaway perspective view 86 of a self-sheathing needle and balloon system 10c in a retracted position 88a. FIG. 14 is a perspective view 90 of a self-sheathing needle and balloon system 10c in an expanded position 88b. Once the compliant balloon 12 is extended 88b from the introducer 16 within a hollow organ HO, the balloon 12 is distended as necessary, and the electrically conductive probes 14 are controllably moved from their undeployed position 44a to a deployed position 44b, whereby the electrically conductive probes 14 extend outwardly into the tissue TI of the hollow organ HO. As described above, the electrically conductive probes 14 are then used for mapping data 26, such as by providing impedance measurements, and can be used to apply energy 36 to ablate the tissue TI surrounding the activated probe needles 14. One or more temperature sensors, such as thermocouples 458, may also be used in conjunction with the probe needles 14, to provide temperature data.

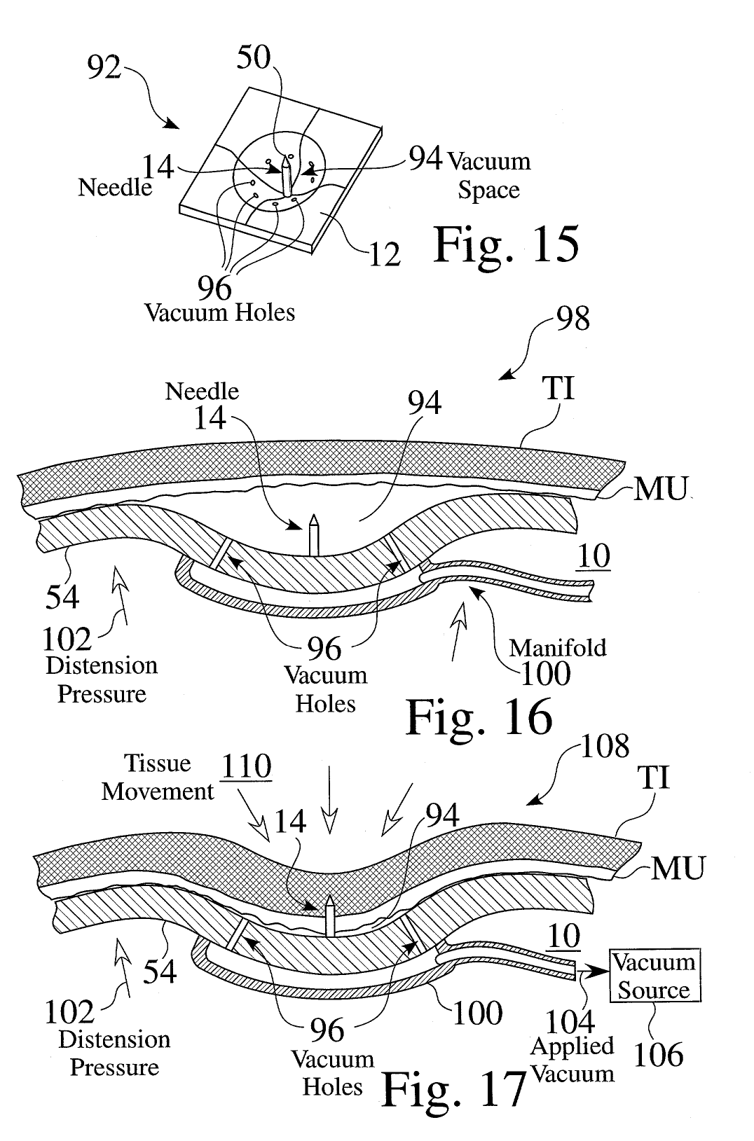

FIG. 15 is a detailed cross sectional view 92 of an alternate ablation probe needle 14 having vacuum actuation for tissue contact. The body of the ablation apparatus 10, such as a compliant balloon 12, includes a recessed area 94 where the electrically conductive needles 14 are located below the surface of the body 12. One or more vacuum holes 96 are also located within the recess area 94, and are interconnected to a vacuum source 106 (FIG. 17). When the body 12 of the ablation apparatus 10 establishes sufficient contact with the hollow organ HO, such as by distending 102 the hollow organ HO, the vacuum source 106 is activated, and the tissue TI of the hollow organ HO is brought into local contact with the probe needles 14.

FIG. 16 is a detailed partial cross sectional view 98 of an ablation structure 10 having a needle 14 located below the surface of the substrate 54 within a recess space 94. One or more vacuum passages 96 extend from the recess space 94 to a vacuum manifold 100, which is connectable to an external vacuum source 106 (FIG. 17). The substrate 54 of the ablation structure 10 establishes sufficient contact with the hollow organ HO, such as by distending 102 the hollow organ HO. As seen in FIG. 16, before vacuum activation, the tissue TI does not contact the probe needle 14. FIG. 17 is a detailed partial cross sectional view 108 of the ablation structure 10 of FIG. 16, having a needle 14 located below the surface of the substrate 54 within a recess space 94, with an applied vacuum 104. When the vacuum source 106 is activated, the tissue TI of the hollow organ HO is moved 110 into local contact with the probe needle 14, such that the needle 14 typically extends through a mucosal layer MU into the tissue TI.

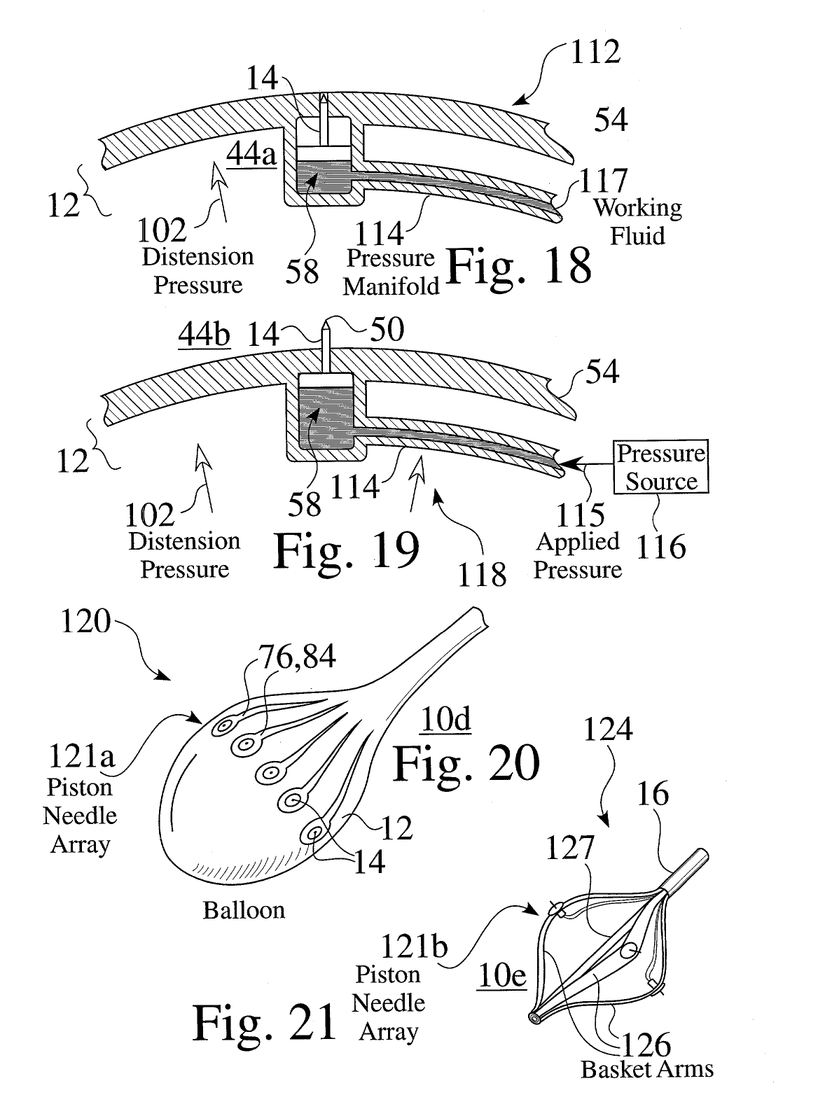

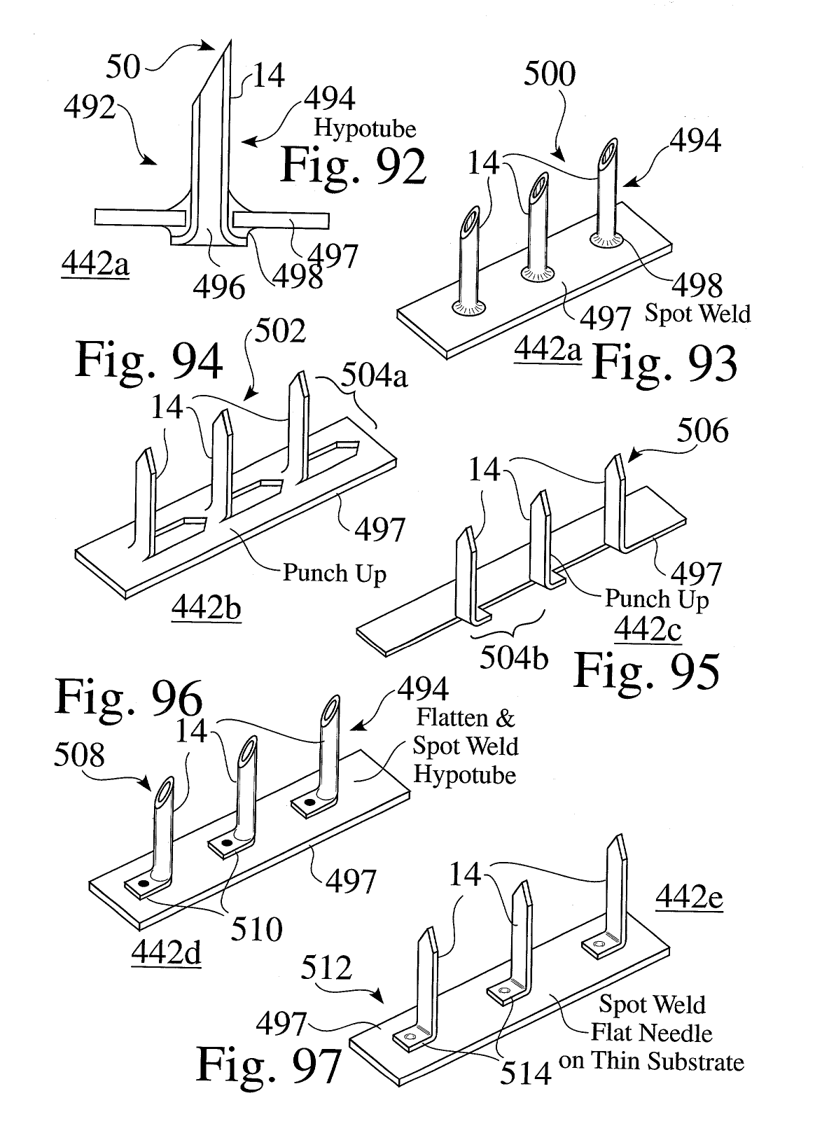

FIG. 18 is a detailed partial cross sectional view 112 of an ablation structure 12 having a hydraulically activatable ablation needle 14, in an unactivated activation 44a. A conduit 58 extends from the hydraulically activatable ablation needle through a pressure manifold 114, which is connectable to an external pressure source 116 (FIG. 19). The substrate 54 of the ablation structure 12 establishes sufficient contact with the hollow organ HO, such as by distending 102 the hollow organ HO. As seen in FIG. 18, before pressure activation 44b, the probe needle 14 is located below the surface of the substrate 54. The working fluid 117 is preferably an aqueous or saline solution 148, and may also preferably be used for localized cooling, such as through a needle port 496 (FIG. 92), or through coolant ports 150 (FIG. 26). FIG. 19 is a detailed partial cross sectional view 118 of the ablation structure 10 of FIG. 18, having a probe needle 14 extending above the surface of the substrate 54 in an activated position 44b, as a result of an applied pressure 115. When the pressure source 116 is activated, the needle 14 extends outwardly from the surface of the substrate 54, typically extending through a mucosal layer MU into tissue TI. As described above, the ablation needle 14, which is electrically connected to the external monitor and control unit 20, is then used for mapping 26 and/or for ablation 36. Temperature sensors 458 are also typically integrated with one or more of the needle structures 14 within an ablation structure 10.

FIG. 20 is a perspective view of a balloon ablation structure 10d having a pressure deployable piston needle array 121a. One or more pressure activatable needles 14, such as shown in FIG. 18 and FIG. 19, are located on the surface of a balloon 12, and may preferably also include convolutions or recessed regions 76,84. In an undeployed position 44a, the balloon structure may be readily inserted or moved within a hollow organ HO, as the tips 50 of the needles 14 do not extend from the balloon 12. In a deployed position 44b, the tips 50 of the needles 14 extend from the balloon 12, and the balloon ablation structure 10d can be used to map 26 or apply energy 36 to a hollow organ HO, through the needles 14 which make electrical contact and thermal contact with tissue TI.

FIG. 21 is a perspective view 124 of a basket ablation structure 10e having a pressure deployable piston needle array 121b. One or more pressure activatable needles 14, such as shown in FIG. 18 and FIG. 19, are located on flexible basket arms 126. The flexible basket arms 126 are connected at opposing ends, and are typically extended and/or retracted by use of a central rod 127. In an unextended position and undeployed position 44a, the basket structure 10e may be readily inserted or moved within a hollow organ HO, as the tips 50 of the needles 14 do not extend from the flexible basket arms 126. In an deployed position 44b, the tips 50 of the needles 14 extend from the flexible basket arms 126, and the basket ablation structure 10e can be used to map 26 or apply energy 36 to a hollow organ HO, such as a stomach ST or a duodenum DU, through the needles 14, which establish electrical contact and thermal contact with tissue TI.

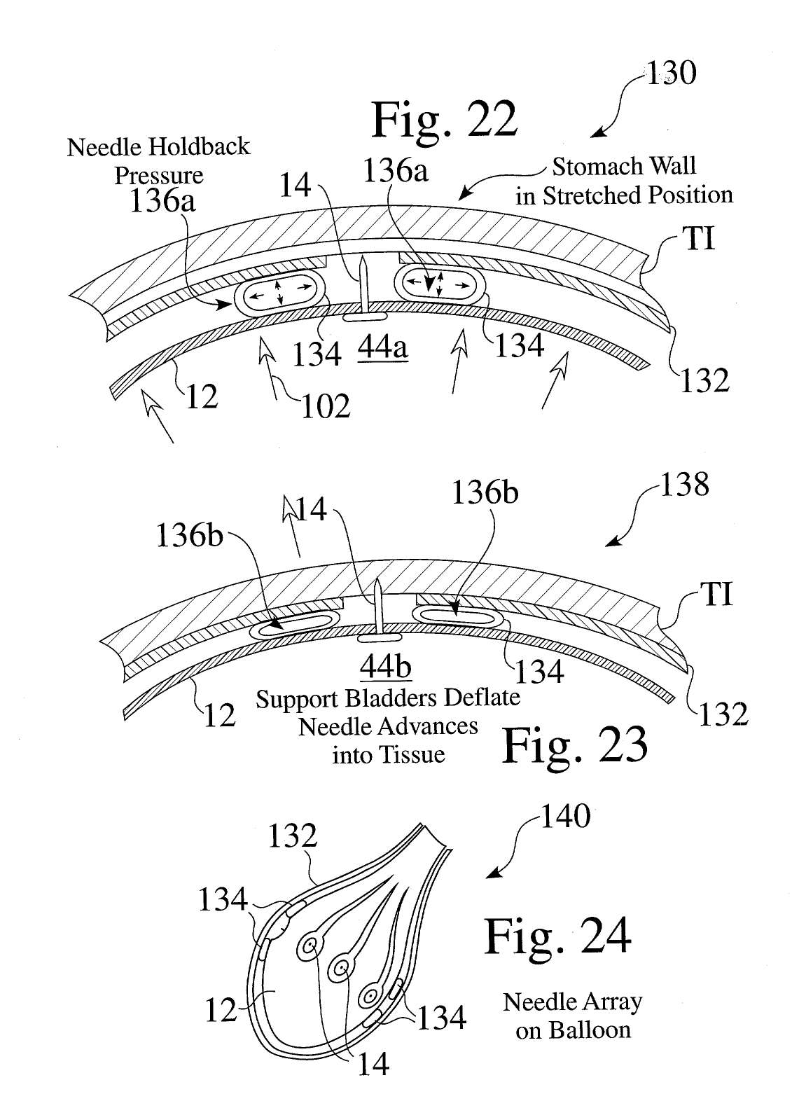

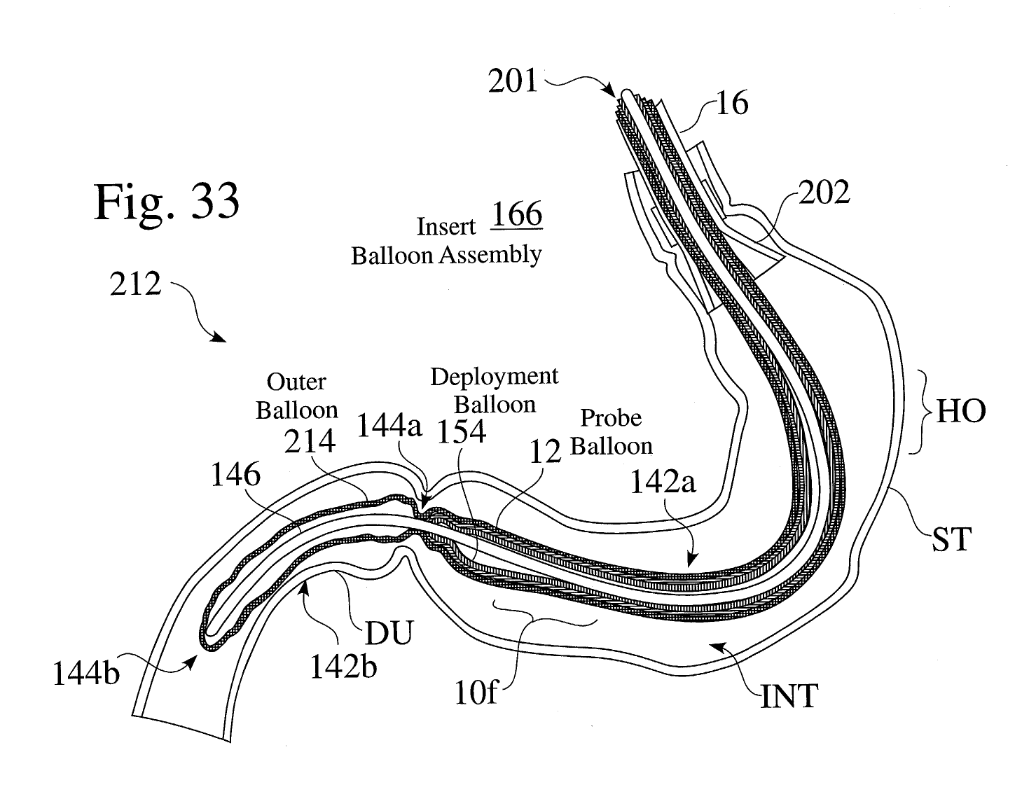

FIG. 22 is a partial cross sectional view 130 of an ablation structure 10 having a distending structure 132, before needle deployment 44b. The outer distending structure 132, such as an outer compliant balloon 214 (FIG. 33), provides a distension force 102 for a hollow organ HO. As seen in FIG. 22, an inner compliant balloon 12 includes one or more electrically conductive needle probes 14, which are located in an undeployed position 44a by inflatable compliant holdback elements 134. When a needle holdback pressure 136a is applied to the inflatable compliant holdback elements 134, the compliant probe balloon 12 is separated from the distending structure 132, and the tips 50 of the probe needles 14 do not make contact with the tissue TI of a distended hollow organ HO.

FIG. 23 is a partial cross sectional view of an ablation structure 10 having a distending structure 132, after needle deployment 44b. FIG. 24 is a partial cutaway view 140 of an ablation structure 10 having an expandable distension balloon structure 132, before needle deployment 132. As seen in FIG. 23, when a second needle pressure 136b is applied to the inflatable compliant holdback elements 134, e.g. such as by deflation, the compliant probe balloon 12 is controllably advanced toward the distending structure 132, and the tips 50 of the probe needles 14 make contact with the tissue TI of a distended hollow organ HO. FIG. 25 is a functional view of an ablation structure 10 having an expandable distension and probe balloon structure 12 and an integrated advancement and retrieval mechanism 146. The compliant balloon 12 shown in FIG. 25 includes a plurality of conductive probes 14, which further comprise fluid ports, such that a conductive fluid 148, such as a saline solution 148, can be dispensed into the ablation areas, such as for thermal cooling and/or for enhanced energy conduction during mapping or ablation processes. The compliant balloon 12 preferably comprises one or more expansion sections 142a,142b, which can be matched to any hollow organ HO for a patient PT, such as to conform to a stomach ST and a duodenum DU, to any portion of the intestinal tract, to a sphincter, or to a uterus. The compliant balloon 12 also preferably comprises one or more anchor sections 144a,144b, either between expansion areas 142, or at the end of the compliant balloon 12.

The integrated advancement and retrieval mechanism 146 shown in FIG. 25 is affixed to the end anchor section 144b, whereby the ablation apparatus 10 may readily be placed within a hollow organ. The integrated advancement and retrieval mechanism 146 is preferably a flexible rod, and may be integrated with the electrical connections 22 and/or process or vacuum connections 24.

FIG. 26 is a partial cross sectional view 152 of a compliant balloon structure 12 having a deployable needle and conductive solution ports 150. An inner compliant balloon 154 is preferably used to move the probe needles 14 between an undeployed position 44a to a deployed position, in which the probes 14 extend from the probe balloon 12. In the compliant balloon structure 12 shown in FIG. 25 and FIG. 26, a conductive saline solution 148 flows from the region between the inner deployment balloon 154 and the probe balloon, and is ejected from probe ports 150.

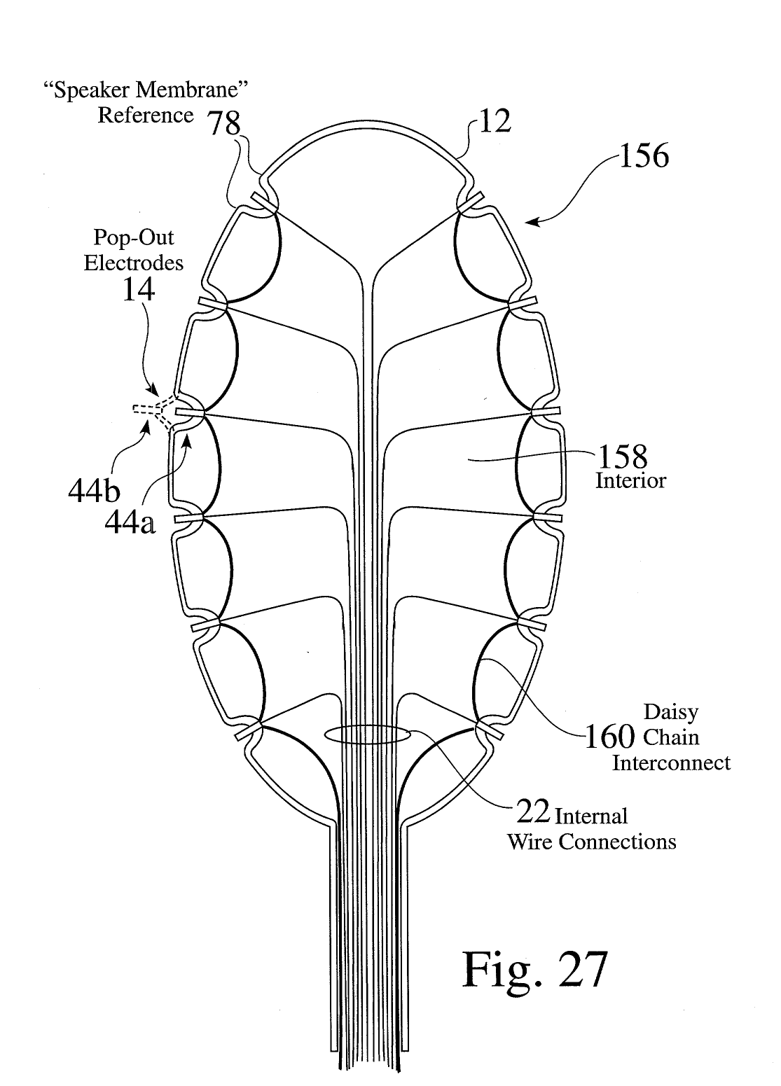

FIG. 27 is a functional cutaway side view 156 of internal electrical connections 22,160 for a compliant probe balloon 12 having deployable probe needle electrodes 14. As described above, some embodiments of the selective ablation system 11 comprise a single compliant balloon 12 having deployable probe needles 14. In alternate embodiments of the selective ablation system 11, a number of staged balloons 12, 154, 214 are integrated to provide distension, deployment, mapping, and ablation. As seen in FIG. 27, each of the probe needle electrodes 14 are deployable from a first unextended position 44a to a second deployed extended position 44b. As well, the compliant probe balloon 12 includes one or more electrical connections 22,160 to the probe needle electrodes 14, such as internal wire connections 22, and/or interconnections 160 between electrodes, e.g. such as a common lead 160. For a compliant probe balloon 12 providing monopolar ablation 36b (FIG. 118), a single power lead 22 is typically attached to a probe needle 14, while an external common electrode 638 (FIG. 118) is typically provided. For a compliant probe balloon 12 providing bipolar ablation 36a, a first power lead 22 is typically attached to a probe needle 14, while a second power lead 22, e.g. such as a ground lead 22, is also provided to the region surrounding each probe needle 14. In some embodiments of the ablation apparatus 10, a saline solution 148 provides an electrical connection to the probe needles 14. In alternate embodiments of the ablation apparatus 10, the compliant balloons further comprise a conductive surface, e.g. such as a conductive film, to provide an electrical connection to the probe needles 14.

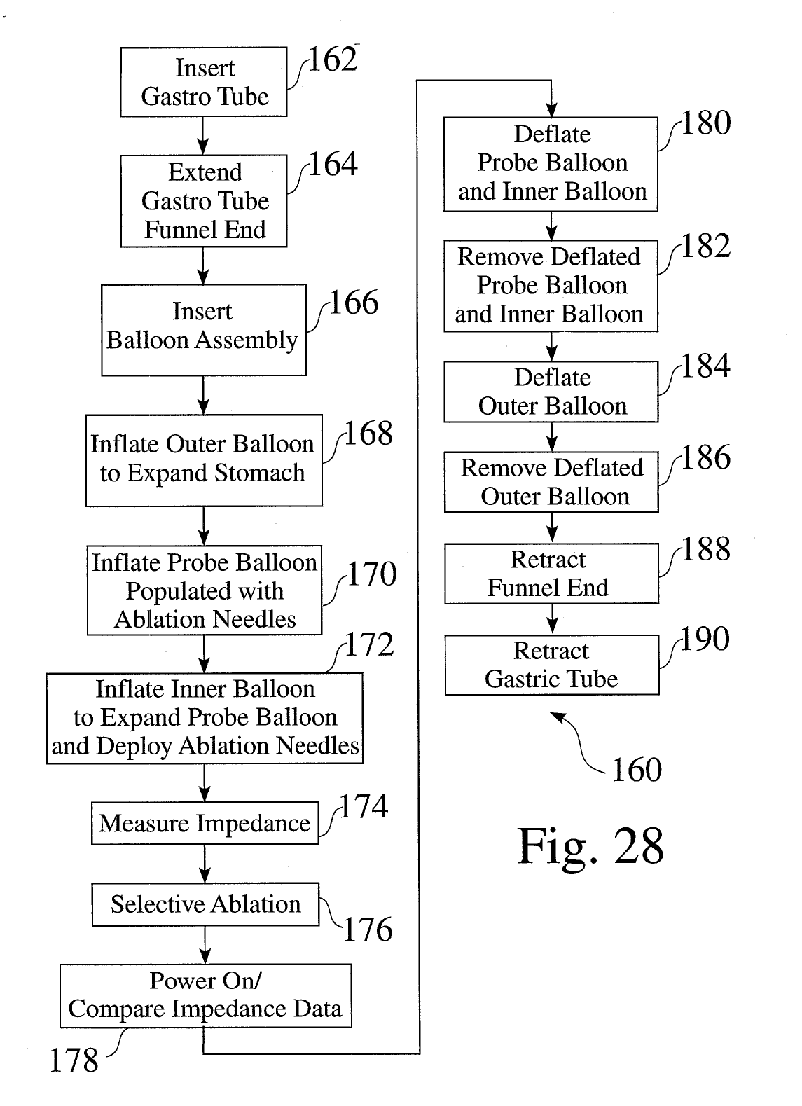

Staged Balloon Ablation Systems. FIG. 28 is a flow diagram of first embodiment of a staged balloon ablation process 160, for a selective ablation system 10f (FIG. 33) comprising an expandable outer distension balloon 214 having a hollow inner region, a second probe balloon assembly comprising a hollow expandable balloon 12 substantially located within the hollow region of the outer balloon 216, at least one deployable electrically conductive needle 14, and an electrical conductor 22 connected to the deployable electrically conductive needle 22 and extending from the interior 158 of the probe balloon 12, and an inner deployment balloon 154 comprising a hollow expandable region substantially located within the interior 158 of the probe balloon 12.

The staged balloon ablation process 160 typically comprises the steps of:

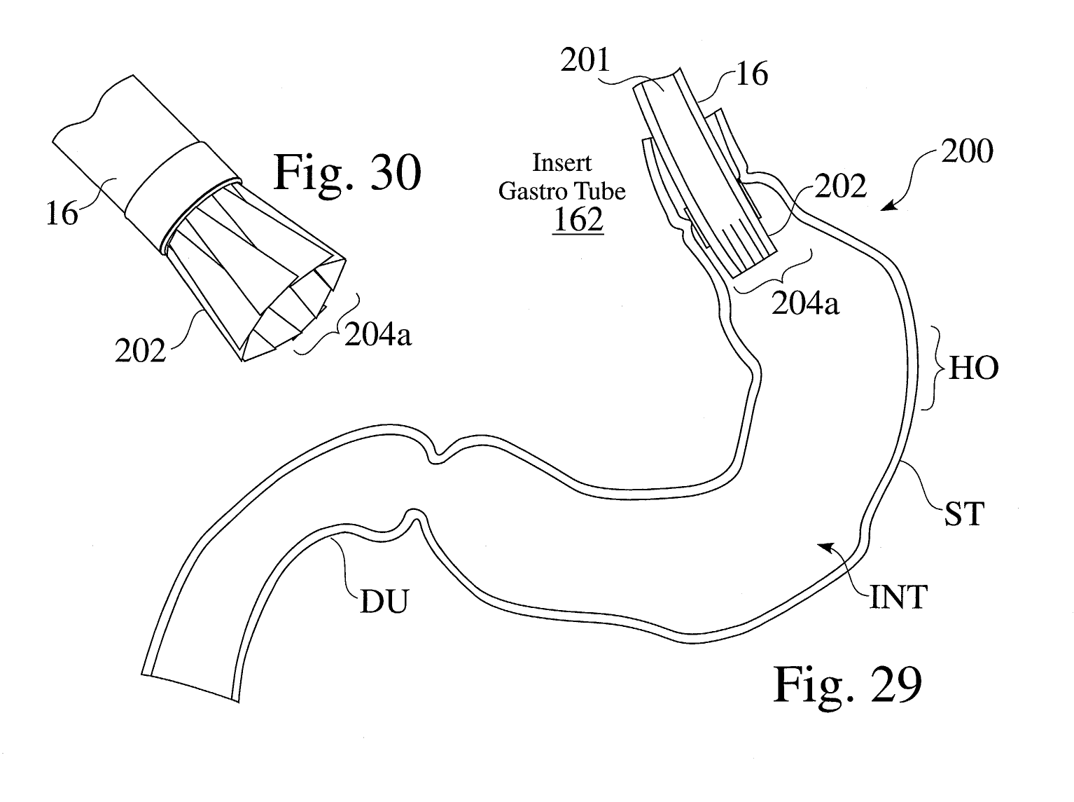

providing an introducer tube 16 having a hollow bore 201 (FIG. 29) between a first end and a second end 202, wherein the second end 202 is preferably expandable;

inserting the second end 202 of the introducer tube 16 into a hollow organ HO, at step 162;

preferably expanding the expandable second end 202, at step 164;

inserting the ablation system 10f through the hollow region 201 of the introducer tube 16 and extending from the second end 202 of the introducer tube 16 into the hollow organ HO, at step 166;

inflating the outer balloon 214 to distend the hollow organ HO, at step 168;

inflating the probe balloon 12 to substantially contact the inflated outer balloon, at step 170; and

inflating the inner balloon 154 to deploy the electrically conductive needles 14 though the outer compliant balloon 214 and into contact with the hollow organ HO, at step 172.

The staged balloon ablation process 160 then typically further comprises the measurement of impedance at the needles 14, at step 174, followed by the selective application of energy 36 through one or more of the needles 14 into the tissue TI of the hollow organ HO, at step 176. Once the ablation step 176 is performed, impedance measurements of the ablated tissue TI may be repeated, and compared to the first impedance data 26 (from step 174), at step 178.

Removal of the deployed ablation system 10f typically comprises the deflation of the deployment balloon 154 and the probe balloon 12, at step 180, removal of the inner deployment balloon 154 and the probe balloon 12, at step 182, deflation of the outer balloon 214, at step 184, removal of the deflated outer balloon 214, at step 186, retraction of the expandable funnel end 202 of the introducer tube 16, at step 188, and the removal of the introducer tube 16, at step 190.

FIG. 29 is a cutaway view 200 which shows the insertion 162 of an introducer tube 16 into the interior region INT of a hollow organ HO, such as a stomach ST, in the first embodiment of a staged balloon ablation process 160. As seen in FIG. 29, the lead end 202 of the introducer tube 16 is in an unexpanded position 204a.

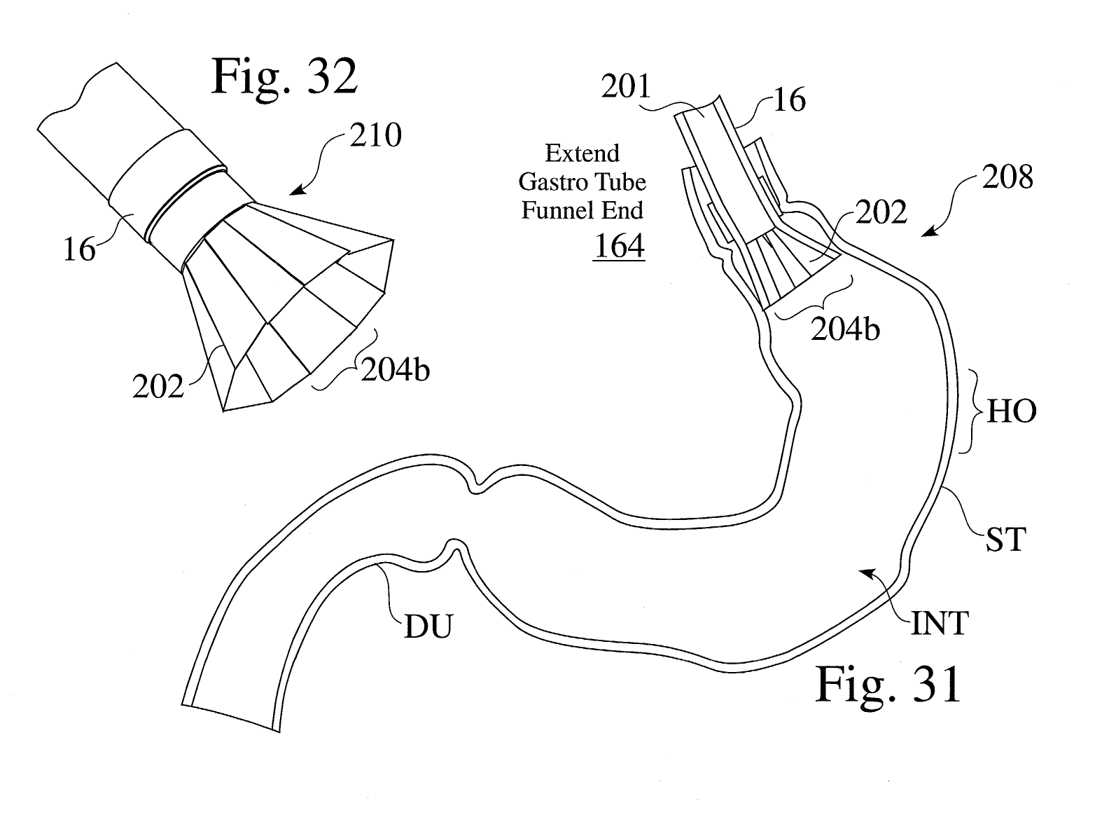

FIG. 30 is a detailed perspective view of an expandable funnel end 202 of an introducer tube 16, in an unexpanded position 204a. FIG. 31 is a cutaway view 208 which shows the expansion 164 of the expandable funnel end 202 of an introducer tube 16, which provides a tapered region for insertion and removal of the ablation apparatus 10f. FIG. 32 is a detailed perspective view 210 of an expandable funnel end 202 of an introducer tube 16, in an expanded position 204b.

FIG. 33 shows the insertion 166 of a staged balloon assembly 10f though a introducer tube 16 in the first embodiment of a staged balloon ablation process 160, wherein the staged balloon assembly 10f preferably includes a flexible internal rod 146, to guide the placement of the staged balloon assembly 10f within the interior INT of the hollow organ HO. As seen in FIG. 33, the outer balloon 214 preferably comprises one or more expansion sections 142a,142b and anchor sections 144a,144b, for accurate placement of the staged balloon assembly 10f within the hollow organ HO, such as within the stomach region ST and duodenum region DU of an intestinal tract.

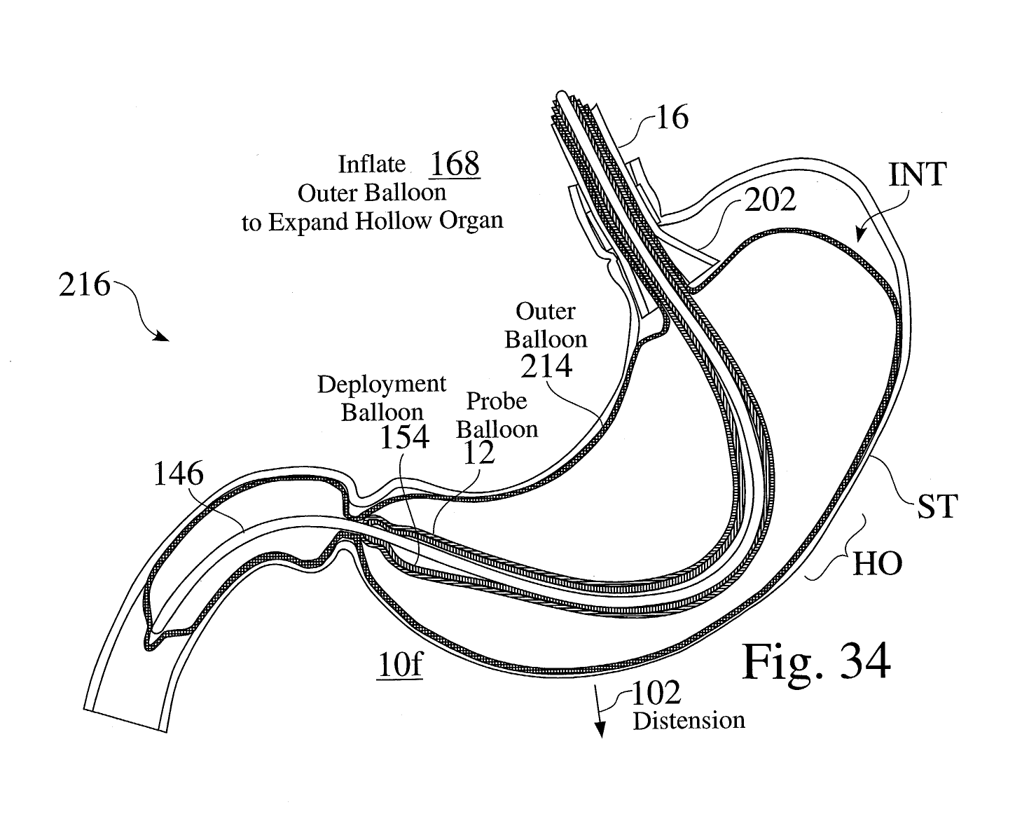

FIG. 34 is a cutaway view 216 which shows inflation 168 of the outer balloon 214 and distension 102 of a stomach ST in the first embodiment of a staged balloon ablation process 160. The expansion sections 142a,142b and anchor sections 144a,144b of the outer balloon 214 provide accurate and secure placement for the ablation assembly 10f. The distension 102 of the hollow organ HO provides access to a large portion of the surface area of the hollow organ HO, which in a non-distended position 602 is a typically pleated structure 600 (FIG. 111), comprising a plurality of pleats PL.

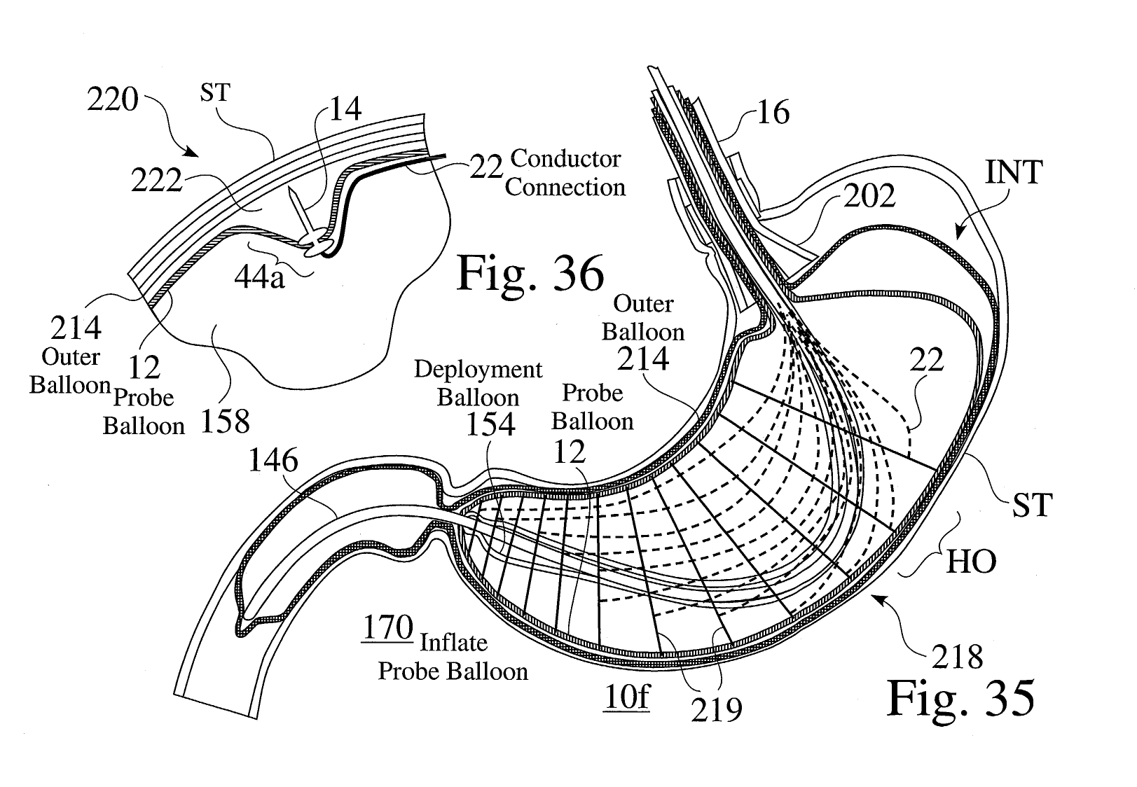

FIG. 35 is a cutaway view 218 which shows inflation 170 of probe needle balloon in the first embodiment of a staged balloon ablation process 160. FIG. 36 is a detailed view 220 of an inflated probe balloon 12 in the first embodiment of a staged balloon ablation process 160. In the probe balloon 12 shown in FIG. 35, electrically conductive connections 22 are provided from the exterior of the system 10f to the probe needles 14, such as for impedance measurement, application of energy, and/or for temperature measurement. While the electrical connections are shown as a plurality of wire leads 22 and conductive ring structures 219, a wide variety of electrical connections 22 can be provided, to one or more of the probe needle regions 14. For example, the probe balloon 12 may preferably comprise a carbon-filled electrically conductive polymeric structure, or may include metallic traces 22, 219. As seen in FIG. 36, while the stomach ST is distended 102 by the outer balloon 214, the probe needles 14 located on the inflated probe balloon 12 are located within the interior 222 of the outer balloon 214, while in an undeployed state 44a.

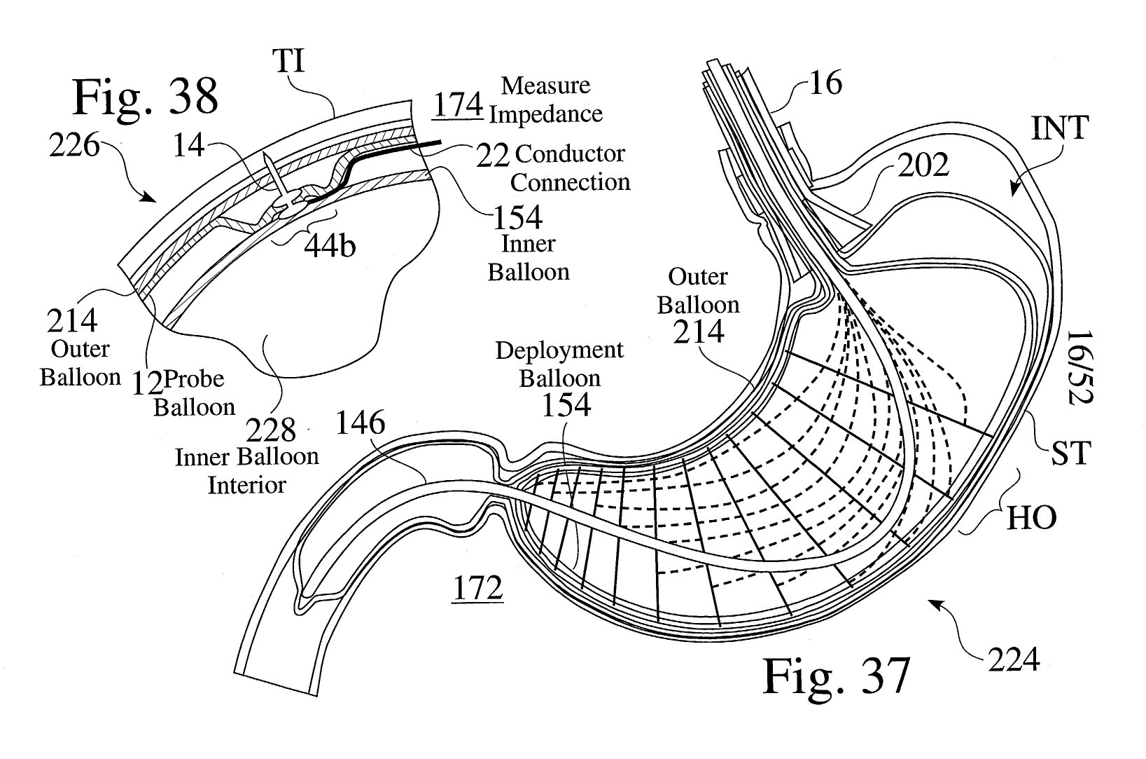

FIG. 37 is a cutaway view 224 which shows inflation 172 of the inner deployment balloon 154 in the first embodiment of a staged balloon ablation process 160. FIG. 38 is a detail view 226 of needle deployment 172 and impedance measurement 174 in the first embodiment of a staged balloon ablation process 160. As seen in FIG. 38, upon inflation 172 of the interior region 228 of the deployment balloon 154, the probe needles 14 located on the inflated probe balloon 12 extend through the outer balloon 214 and into the distended tissue TI, while in a deployed state 44b.

In some embodiments of the probe balloon 12 which is used in a stomach ST, the deployed probe needles 14 allow a physician to identify focal nerve sites in the stomach ST and/or upper duodenum DU that are associated with producing sensations of hunger and satiety.

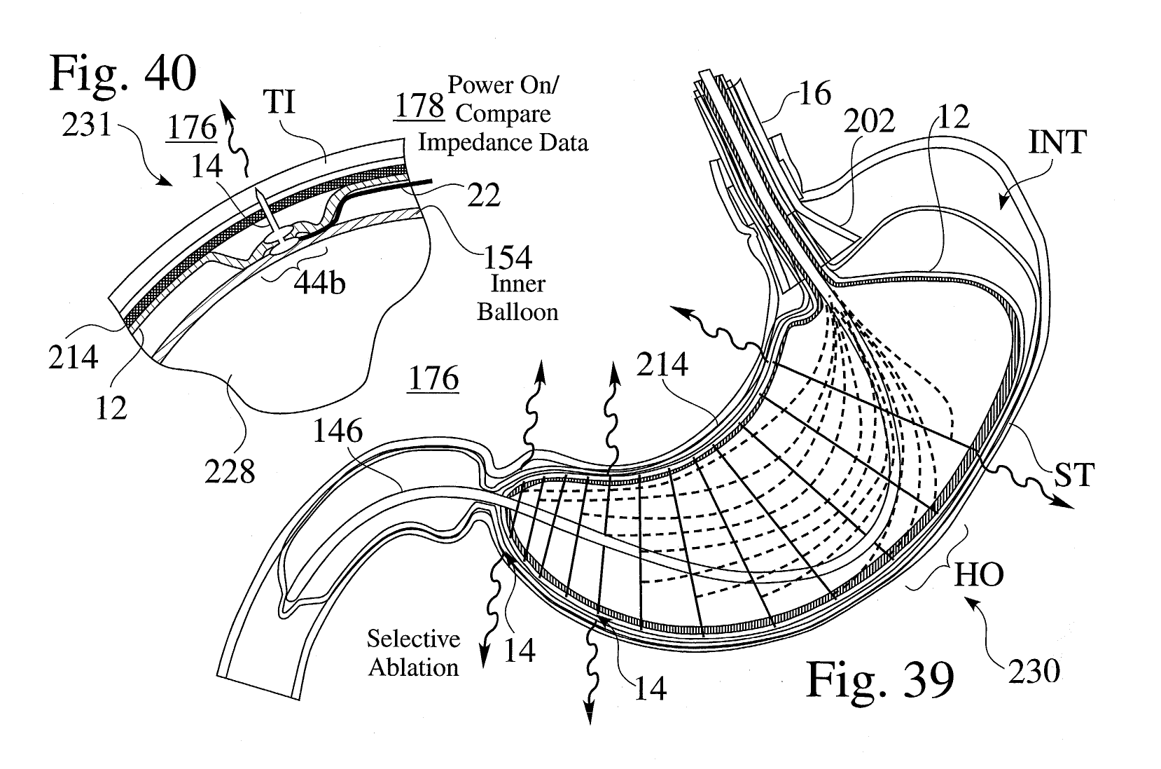

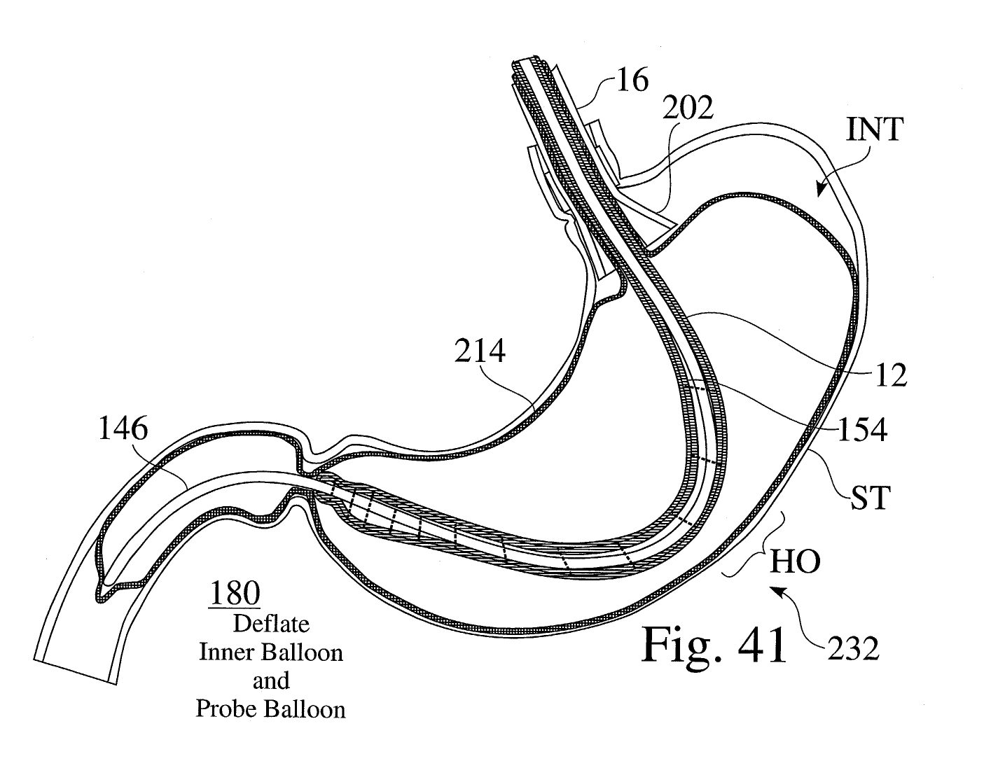

FIG. 39 is a cutaway view 230 which shows selective ablation 176 through deployed probe needles 14 in the first embodiment of a staged balloon ablation process 160. FIG. 40 is a detail view 231 of selective ablation 176 and subsequent impedance measurement 178 through a deployed needle 14 in the first embodiment of a staged balloon ablation process 160.

In some embodiments of the probe balloon 12 which is used in a stomach ST, the deployed probe needles 14 allow a physician to selectively ablate 36 focal nerve sites in the stomach ST and/or upper duodenum DU that are associated with producing sensations of hunger and satiety. As well, the ablation energy 36 can be used to shrink selected portions of the innermost oblique muscle and circular muscle layers of the stomach ST. This can be performed in a physician's office, using local anesthesia. Shrinkage of these muscles produces a feeling of satiety that enhances the patient's effort to restrict caloric intake.

FIG. 41 is a cutaway view 232 which shows deflation 180 of the inner deployment balloon 154 and the probe balloon 12 in the first embodiment of a staged balloon ablation process 160. The balloon deflation 180 moves the probe needles 14 to an undeployed state 44a, whereby the inner deployment balloon 154 and the probe balloon 12 are readily and safely removed, preventing further contact between the tips 50 of the needle probes 14 and the hollow organ HO.

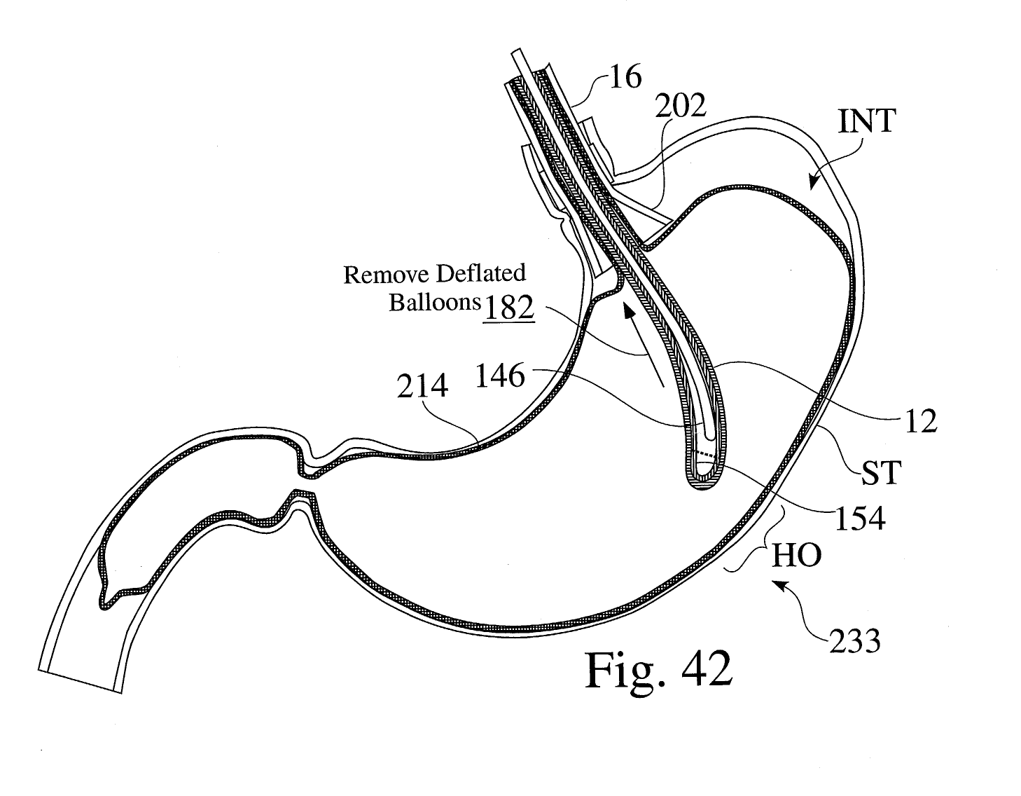

FIG. 42 is a cutaway view 233 which shows the removal of the deflated inner deployment balloon 154 and the probe balloon 12 in the first embodiment of a staged balloon ablation process 160. The introducer tube 16 and the outer balloon 214 provide a smooth transition region by which the center rod 146, the deflated inner deployment balloon 154, and the probe balloon 12 are readily guided during removal 180.

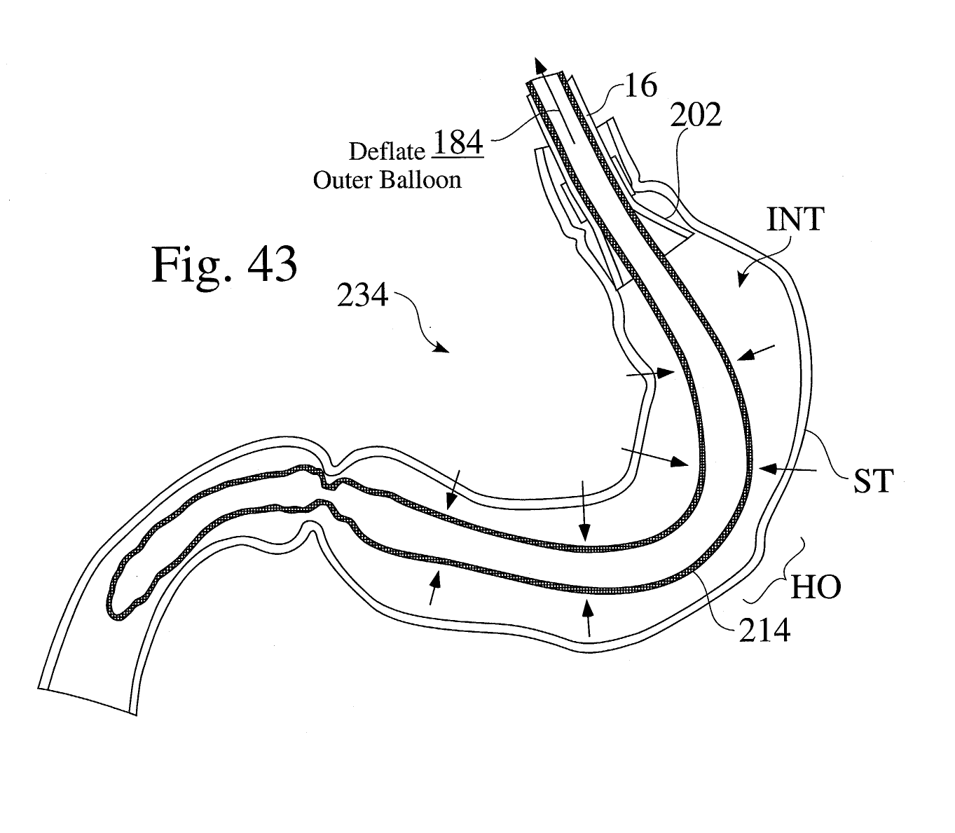

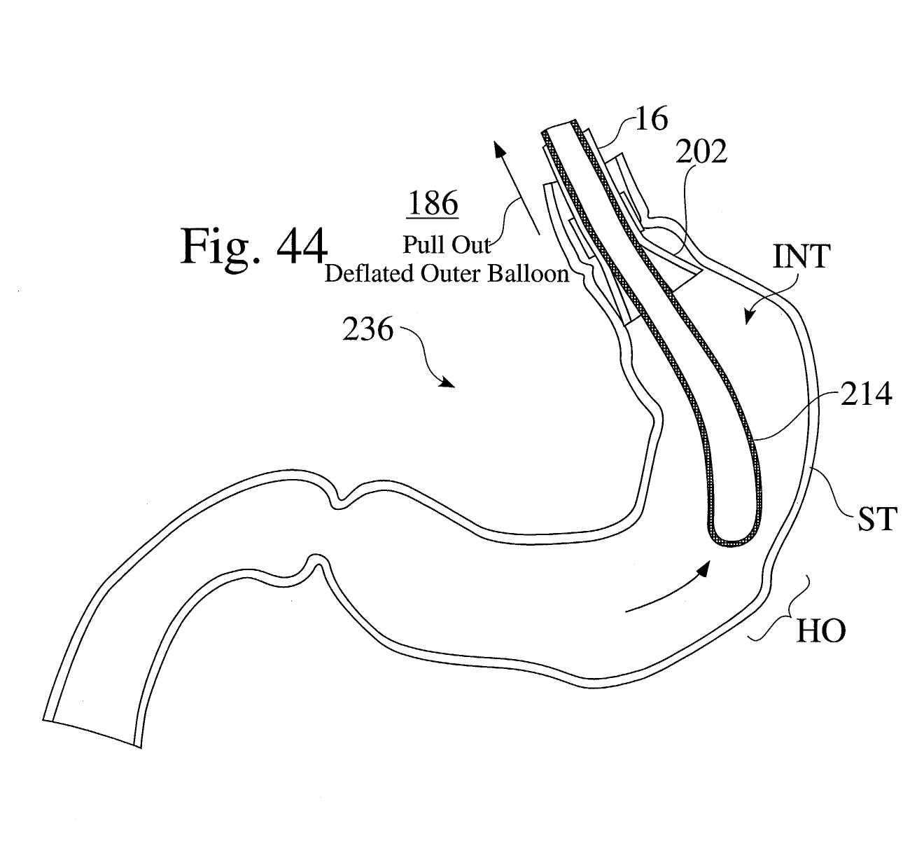

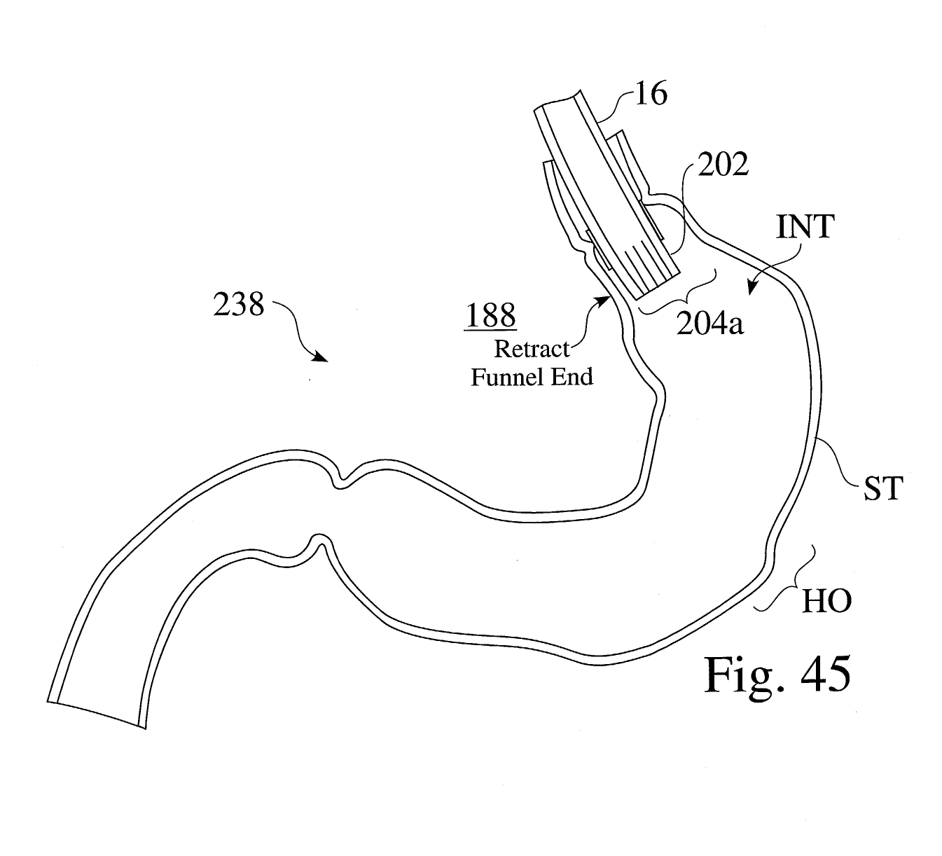

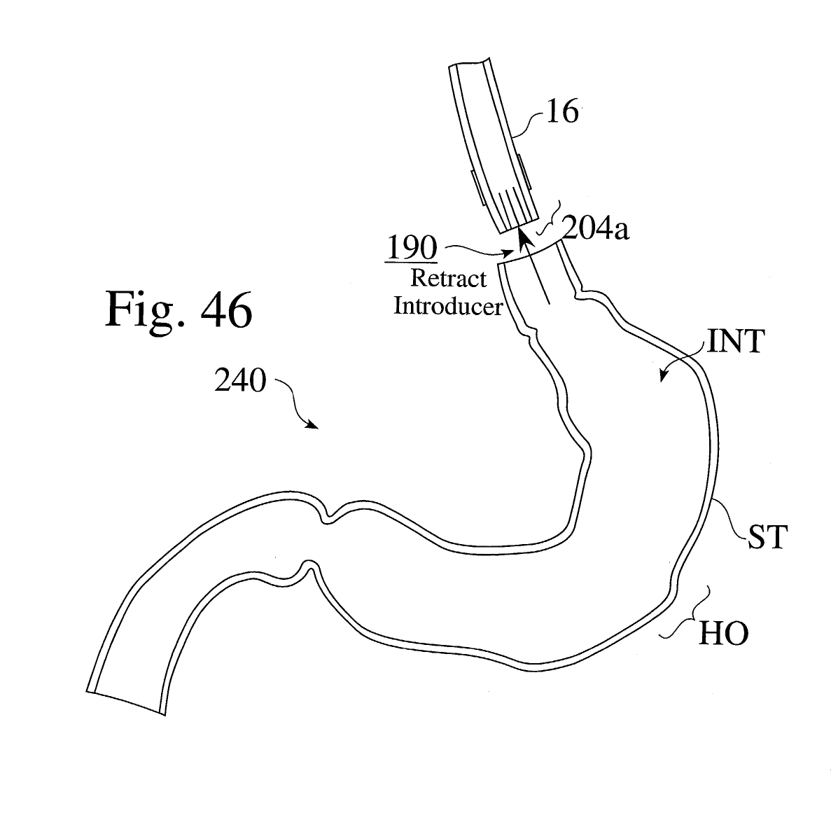

FIG. 43 is a cutaway view 234 which shows the deflation 184 of the outer balloon 214 in the first embodiment of a staged balloon ablation process 160. FIG. 44 is a cutaway view 236 which shows the removal 186 of the deflated outer balloon 214 from the interior INT of the hollow organ HO in the first embodiment of a staged balloon ablation process 160. The expanded funnel end 202 of the introducer tube 16 provides a smooth transition region by which the deflated outer balloon 214 is readily guided during removal 186. FIG. 45 is a cutaway view 238 which shows funnel-end retraction 188 for the introducer tube 16 in the first embodiment of a staged balloon ablation process 160. FIG. 46 is a cutaway view 240 which shows the removal 190 of the introducer 16 in the first embodiment of a staged balloon ablation process 16.

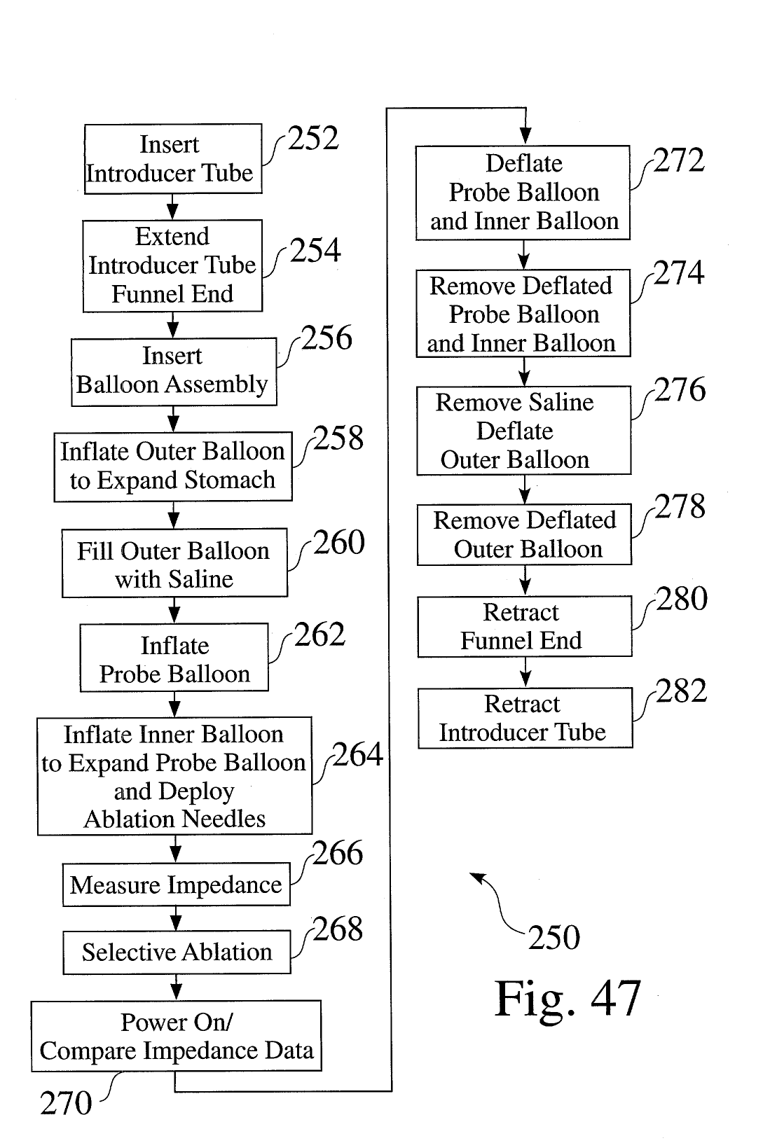

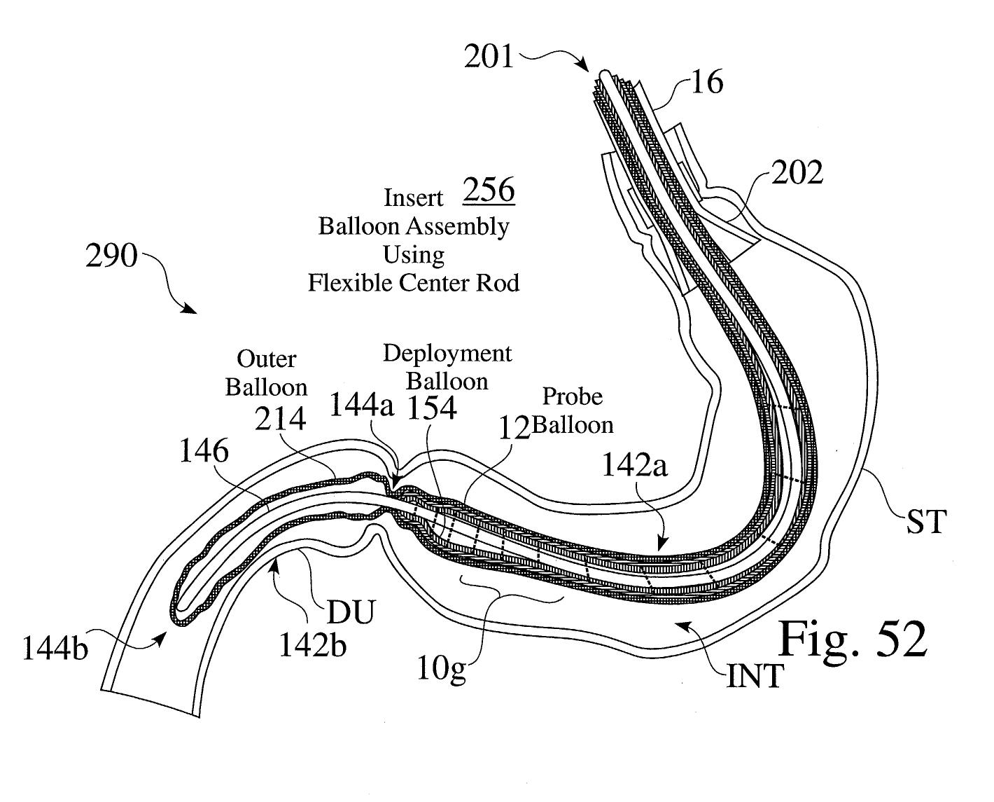

Saline Conductor Structure & Process. FIG. 47 is a flow diagram of second embodiment of a staged balloon ablation process 250, for a selective ablation system log (FIG. 52) comprising an expandable outer distension balloon 214 having a hollow inner region, a second probe balloon assembly comprising a hollow expandable balloon 12 substantially located within the hollow region of the outer balloon 216, at least one deployable electrically conductive needle 14, and means for establishing a fluid-based electrical connection 148 to the deployable electrically conductive needle 14 through the interior 158 of the probe balloon 12, and an inner deployment balloon 154 comprising a hollow expandable region substantially located within the interior 158 of the probe balloon 12.

In some embodiments of the selective ablation system 10g, the probe balloon 12 comprises as much as or more than fifty, seventy five, or one hundred probe needles 14. As well, in some embodiments of the selective ablation system log to be used for the ablation of a stomach ST, the probe needles 14 in generally located to coincide with designated areas within a stomach ST, such as within the upper stomach and/or the lower stomach or duodenum DU.

The staged balloon ablation process 250 typically comprises the steps of:

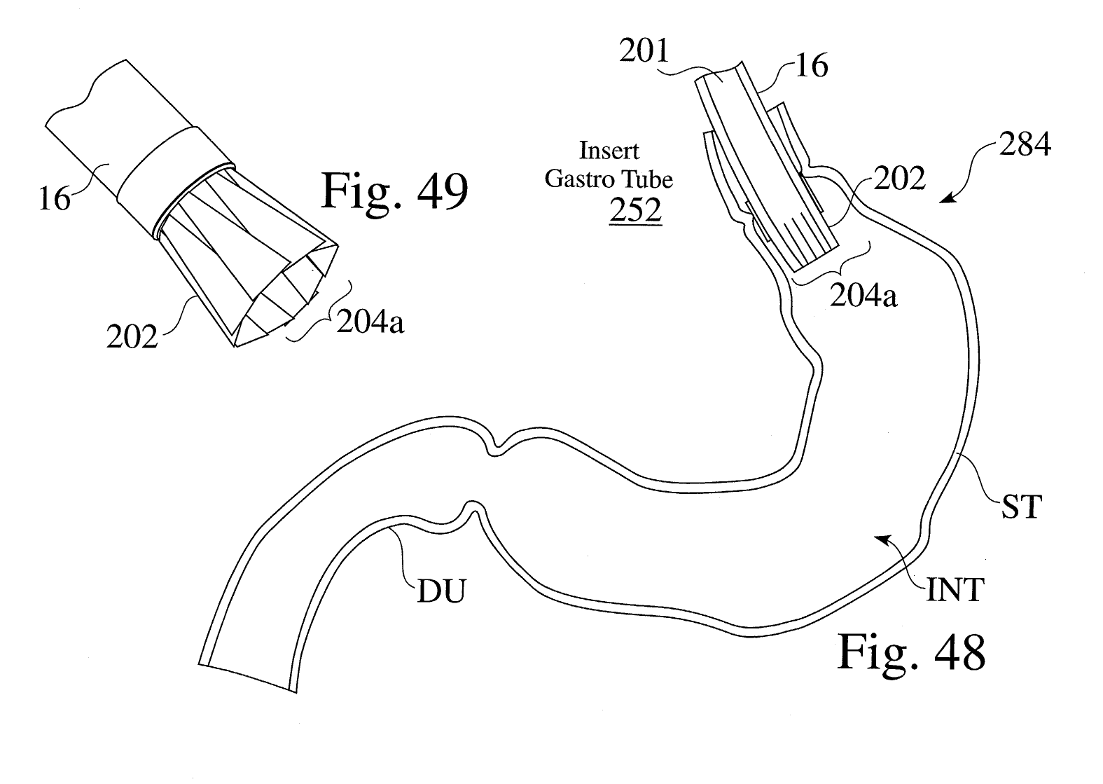

providing an introducer tube 16 having a hollow bore 201 (FIG. 48) between a first end and a second end 202, wherein the second end 202 is preferably expandable;

inserting the second end of the introducer tube 16 into a hollow organ HO, at step 252;

preferably expanding the expandable second end 202 of the introducer tube 16, at step 254;

inserting the ablation system log through the hollow region 201 of the introducer tube 16 and extending from the second end 202 of the introducer tube 16 into the hollow organ HO, at step 256;

inflating the outer balloon 214 to distend the hollow organ HO, at step 258;

introducing a conductive solution, such as saline 148, into the outer balloon 214, at step 260;

inflating the probe balloon 12 to substantially contact the inflated outer balloon 214, at step 260; and

inflating the inner balloon 154 to deploy electrically conductive needles 14 located on the probe balloon 12 though the outer compliant balloon 214 and into contact with the hollow organ HO, at step 264.

The staged balloon ablation process 250 then typically further comprises the measurement of impedance at the needles 14, at step 266, followed by the selective application of energy 36 through one or more of the needles 14 into the tissue TI of the hollow organ HO, at step 268. Once the ablation step 268 is performed, impedance measurements of the ablated tissue TI may be repeated, and compared to the first impedance data, at step 270.

Removal of the deployed ablation system log typically comprises the deflation of the deployment balloon 154 and the probe balloon 12, at step 272, removal of the deflated deployment balloon 154 and probe balloon 12, at step 274, removal of saline 148 and deflation of the outer balloon 214, at step 276, removal of the deflated outer balloon 214, at step 278, retraction of the expandable end 202 of the introducer tube 16, at step 280, and the removal of the introducer tube 16, at step 282.

FIG. 48 is a cutaway view 284 which shows the insertion 252 of an introducer tube 16 into the interior region INT of a hollow organ HO, such as a stomach ST, in the second embodiment of a staged balloon ablation process 250. As seen in FIG. 48, the lead end 202 of the introducer tube 16 is in an unexpanded position 204a. FIG. 49 is a detailed perspective view of an expandable funnel end 202 of an introducer tube 16, in an unexpanded position 204a.

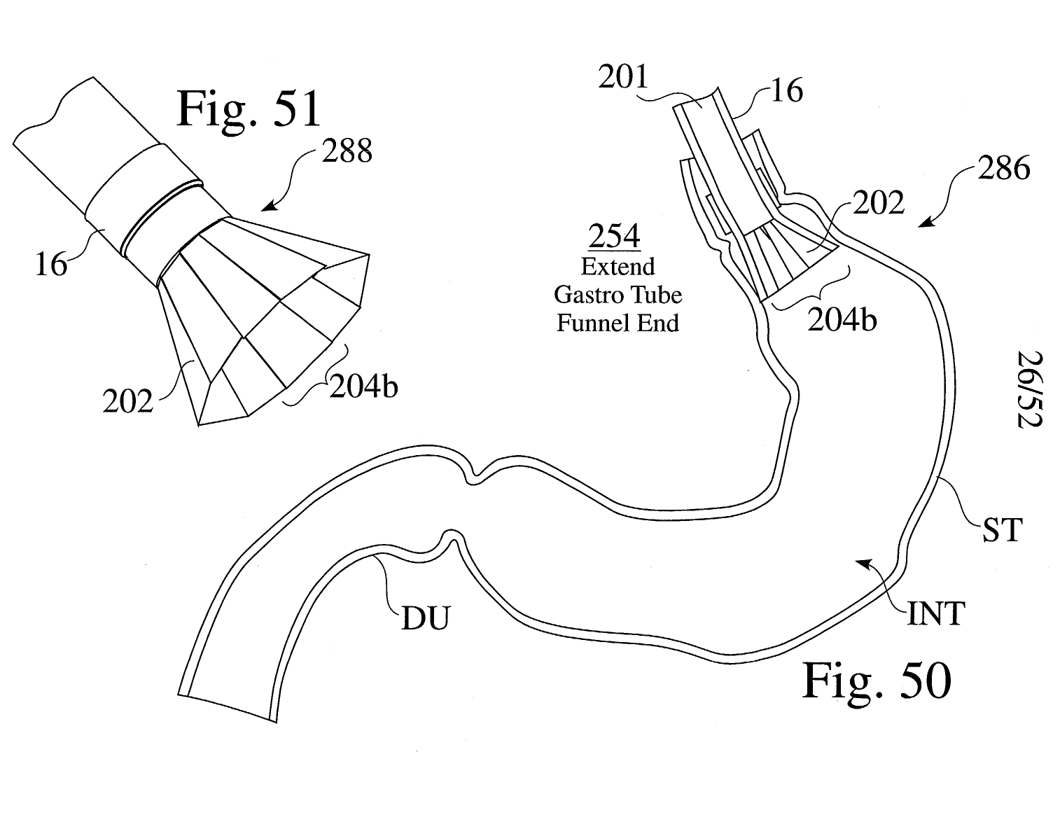

FIG. 50 is a cutaway view 286 which shows the expansion 254 of the expandable funnel end 202 of an introducer tube 16, which provides a tapered region for insertion and removal of the ablation apparatus 10g. FIG. 51 is a detailed perspective view 288 of an expandable funnel end 202 of an introducer tube 16, in an expanded position 204b.

FIG. 52 shows the insertion 256 of a staged balloon assembly log though a introducer tube 16 in the second embodiment of a staged balloon ablation process 250, wherein the staged balloon assembly log preferably includes a flexible internal rod 146, to guide the placement of the staged balloon assembly log within the interior INT of the hollow organ HO. As seen in FIG. 52, the outer balloon 214 preferably comprises one or more expansion sections 142a,142b and anchor sections 144a,144b, for accurate placement of the staged balloon assembly log within the hollow organ HO.

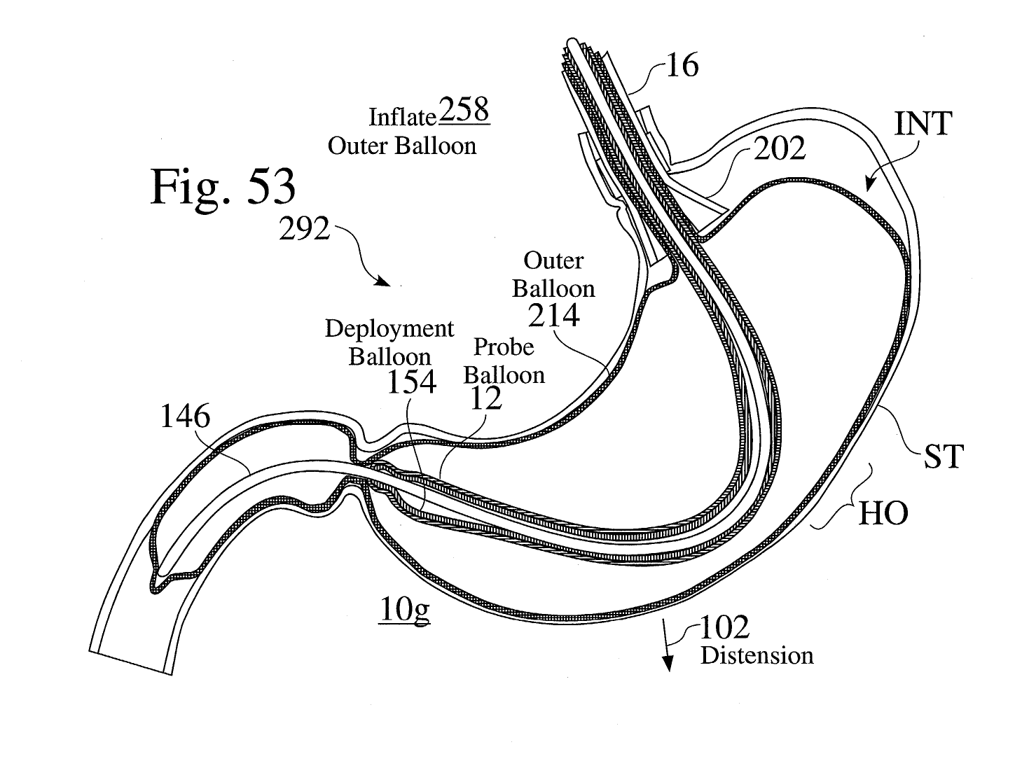

FIG. 53 is a cutaway view 292 which shows inflation 258 of the outer balloon and distension 102 of a hollow organ HO in the second embodiment of a staged balloon ablation process 250. The expansion sections 142a,142b and anchor sections 144a,144b of the outer balloon 214 provide accurate and secure placement for the ablation assembly 10g. The distension 102 of the hollow organ HO provides access to a large portion of the surface area of the hollow organ HO, which in a non-distended position 602 is a typically pleated structure 600 (FIG. 111), comprising a plurality of pleats PL.

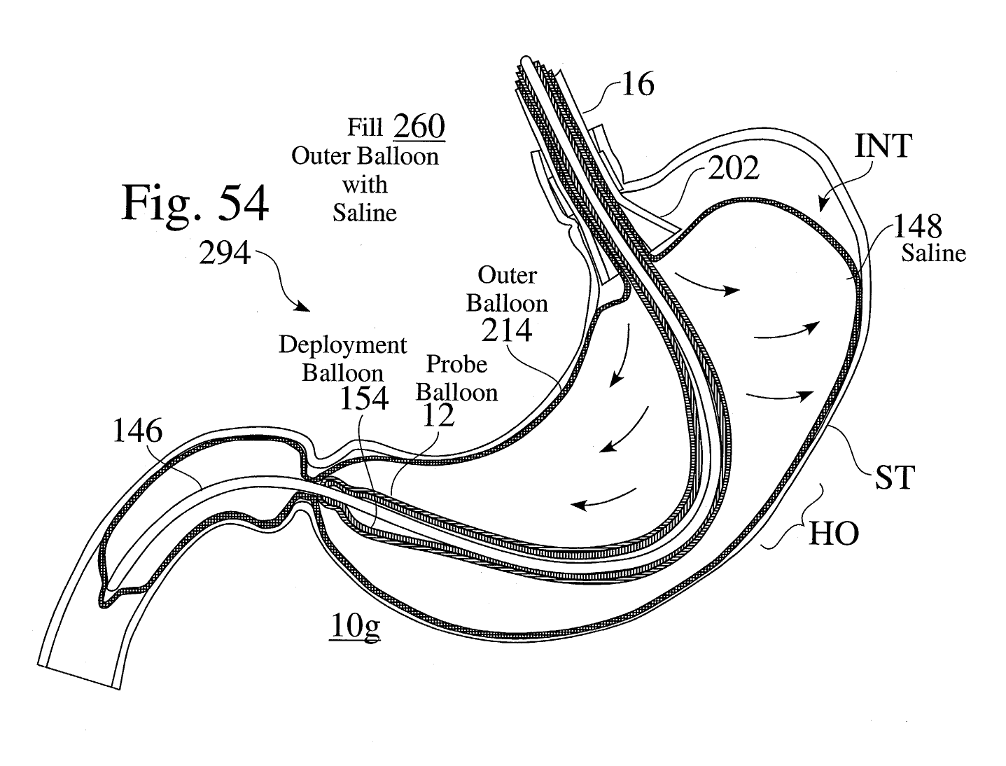

FIG. 54 is a cutaway view 294 which shows introduction 260 of a conductive solution 148, such as saline 148, into the interior region 22 of the outer balloon 214 in the second embodiment of a staged balloon ablation process 250. As described above, the saline 148 can be used to establish electrical connections to one or more of the probes, such as for the application of ablation energy 36, and/or for the measurement of impedance 26. As well, Saline 148 is preferably used in some selective ablation structures 10 for ablation zone cooling, such that the local tissue TI surrounding a needle probe 14 is not over-heated during an ablation process 36.

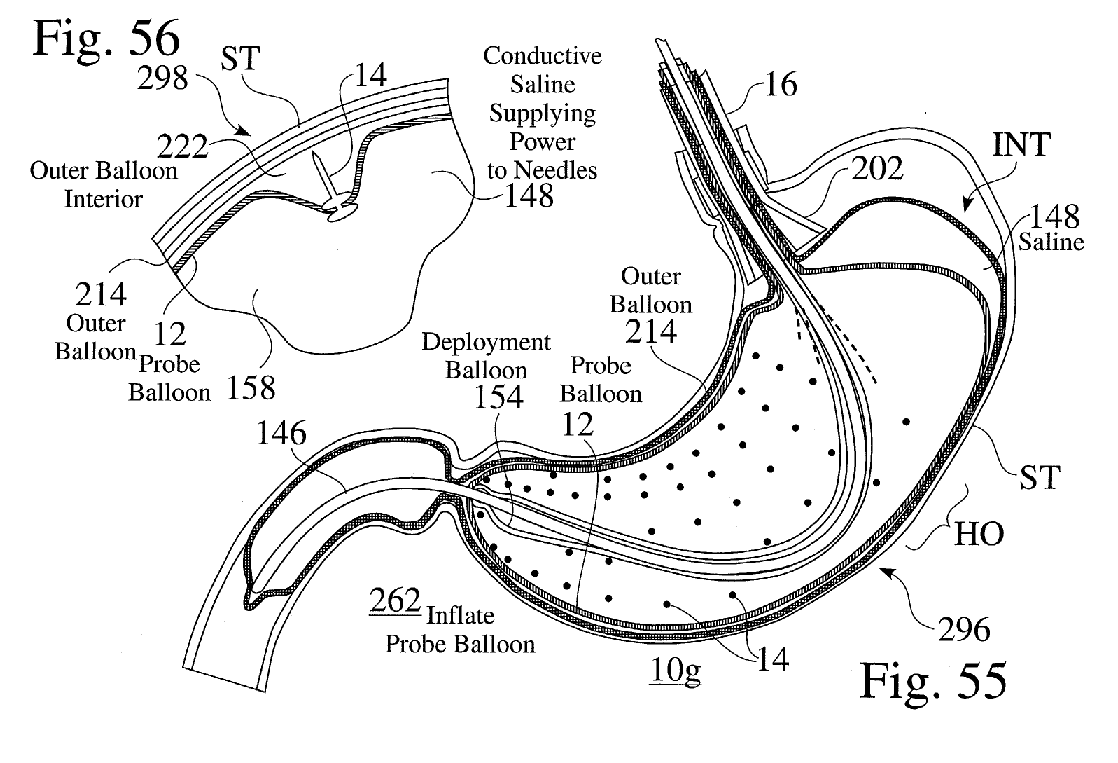

FIG. 55 is a cutaway view 296 which shows inflation 262 of probe needle balloon 12 in the second embodiment of a staged balloon ablation process 250. FIG. 56 is a detailed view 298 of an inflated probe balloon 12 in the second embodiment of a staged balloon ablation process 250.

In the probe balloon 12 shown in FIG. 55, electrically conductive connections 22 are established from the exterior of the system 10g to the probe needles 14 by use of the electrically conductive solution 148, such as for impedance measurement, application of energy, and/or for temperature measurement. While the electrical connections are shown as a saline connection 22, other electrical connections, such as wire leads 22 or conductive ring structures 219 may also be provided, to one or more of the probe needle regions 14. For example, the probe balloon 12 may preferably comprise a carbon-filled polymeric structure or layer, or may include metallic traces 22, 219. Furthermore, the surface of the probe balloon 12 may comprise a textured or patterned surface, such as to promote electrical contact between the probes 14 and the conductive solution 148.

As seen in the detail view 298 of FIG. 56, while the stomach ST is distended by the outer balloon 214, the probe needles 14 located on the inflated probe balloon 12 are located within the interior 222 of the outer balloon 214, while in an undeployed state 44a.

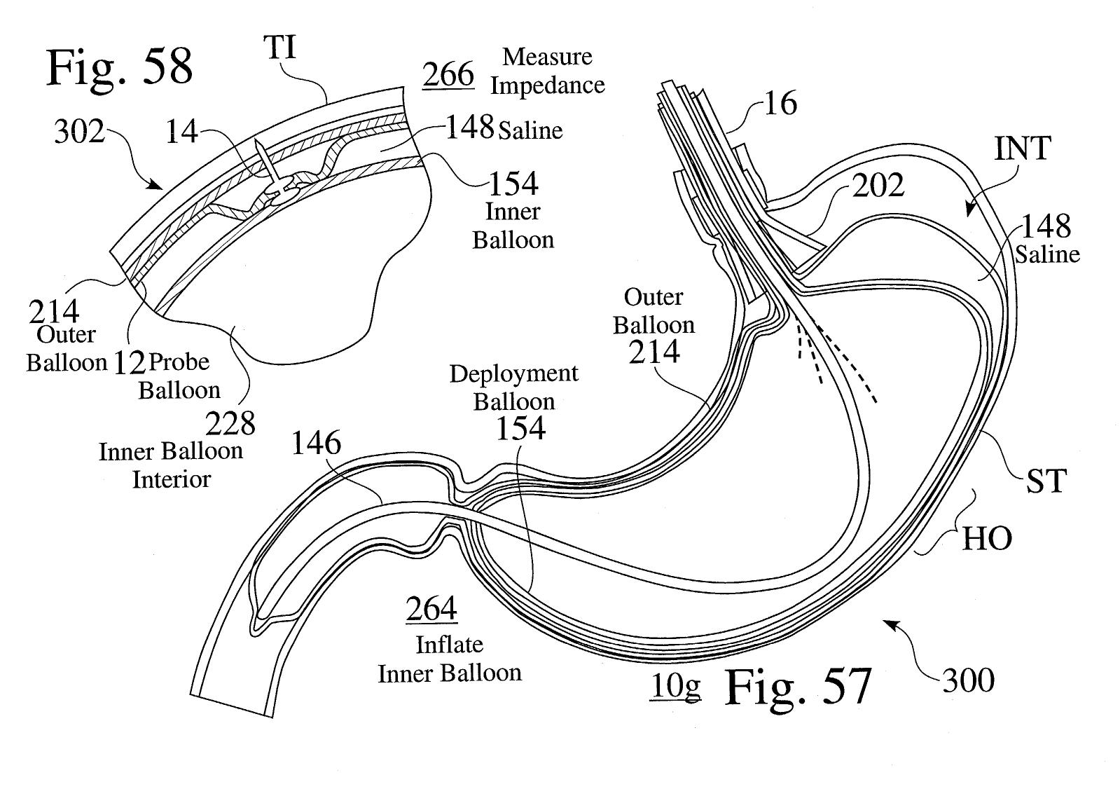

FIG. 57 is a cutaway view 300 which shows inflation 264 of the inner deployment balloon 154 in the second embodiment of a staged balloon ablation process 250. FIG. 58 is a detail view 302 of needle deployment 264 and impedance measurement 266 in the second embodiment of a staged balloon ablation process 250. As seen in FIG. 58, upon inflation 264 of the interior region 228 of the deployment balloon 154, the probe needles 14 located on the inflated probe balloon 12 extend through the outer balloon 214 and into the distended tissue TI, while in a deployed state 44b.

FIG. 59 is a cutaway view 304 which shows selective ablation 268 through deployed needles 14 in the second embodiment of a staged balloon ablation process 250. FIG. 60 is a detail view 306 of selective ablation 268 and subsequent impedance measurement 270 through a deployed needle 14 in the second embodiment of a staged balloon ablation process 250.

In some embodiments of the probe balloon 12 which is used in a stomach ST, the deployed probe needles 14 allow a physician to selectively ablate 36 focal nerve sites in the stomach ST and/or upper duodenum DU that are associated with producing sensations of hunger and satiety. As well, the ablation energy 36 can be used to shrink selected portions of the innermost oblique muscle and circular muscle layers of the stomach ST. This can be performed in a physician's office, using local anesthesia. Shrinkage of these muscles produces a feeling of satiety that enhances the patient's effort to restrict caloric intake.

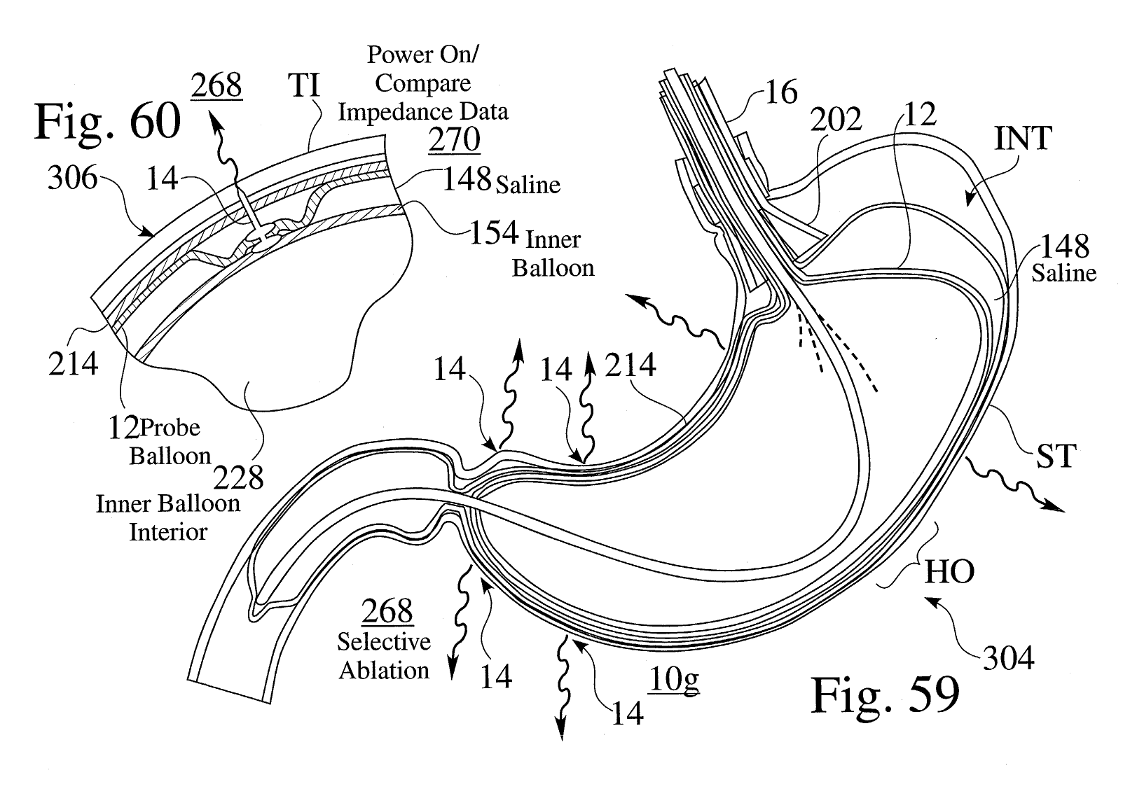

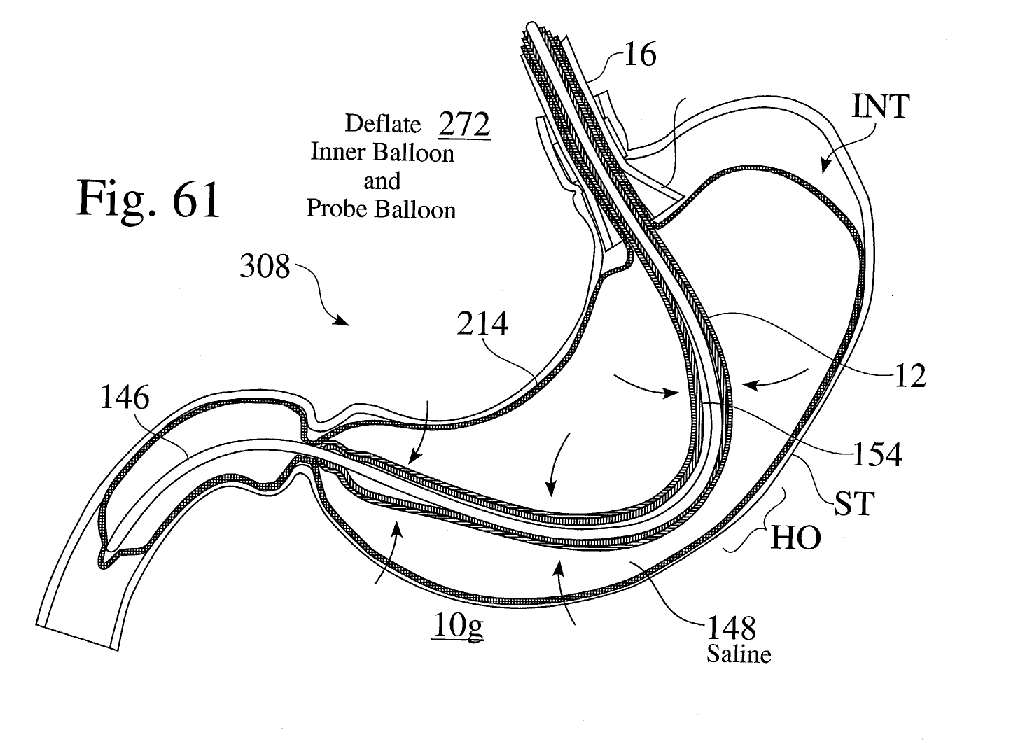

FIG. 61 is a cutaway view 308 which shows deflation 272 of the inner deployment balloon 154 and the probe balloon 12 in the second embodiment of a staged balloon ablation process 250. The balloon deflation 272 returns the probe needles 14 to an undeployed state 44a, whereby the inner deployment balloon 154 and the probe balloon 12 are readily and safely removed, preventing further contact between the tips 50 of the needle probes 14 and the hollow organ HO. The balloon deflation 272 may preferably be accompanied by the introduction of more saline 148 into the interior region 222 of the outer balloon 214, such as to promote deflation of the inner deployment balloon 154 and the probe balloon 12.

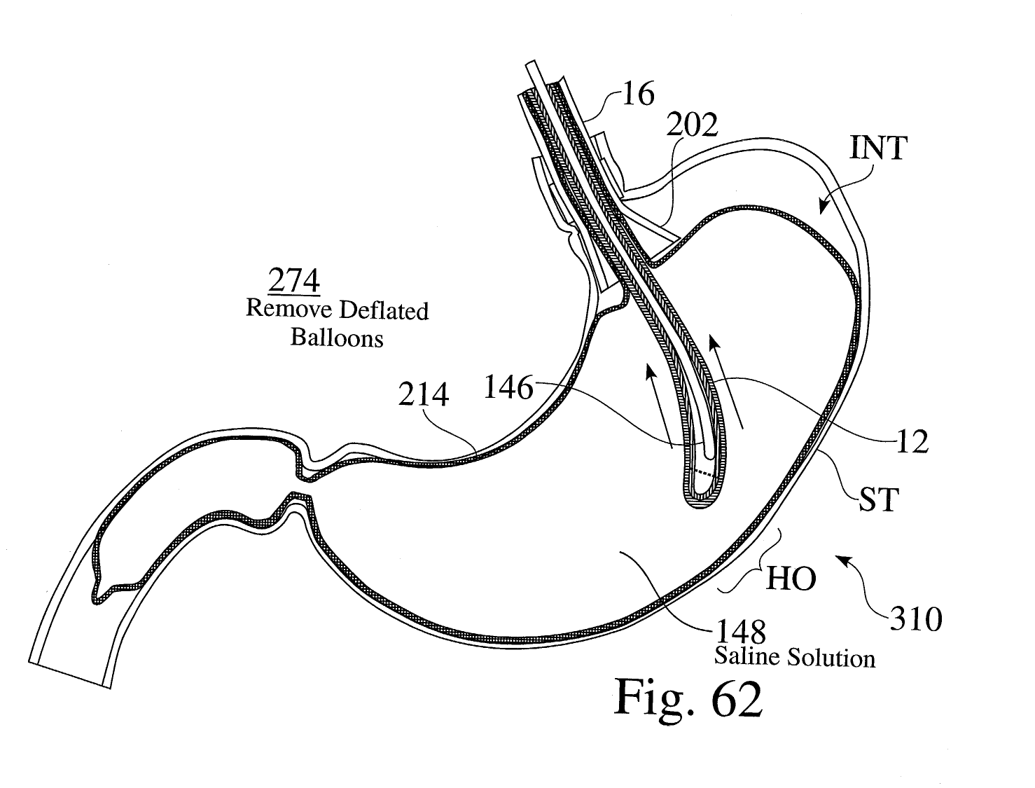

FIG. 62 is a cutaway view 310 which shows the removal 274 of the deflated inner deployment balloon 154 and the probe balloon 12 in the second embodiment of a staged balloon ablation process 250. The introducer tube 16 and the outer balloon 214 provide a smooth transition region by which the center rod 146, the deflated inner deployment balloon 154, and the probe balloon 12 are readily guided during removal 274.

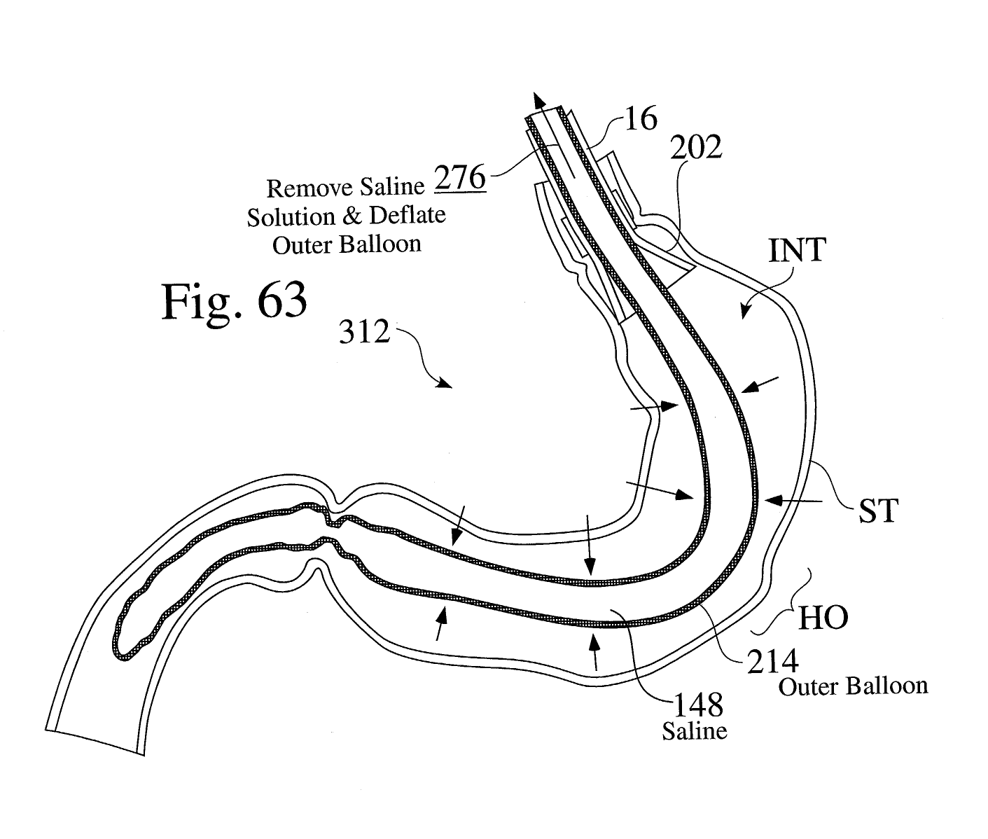

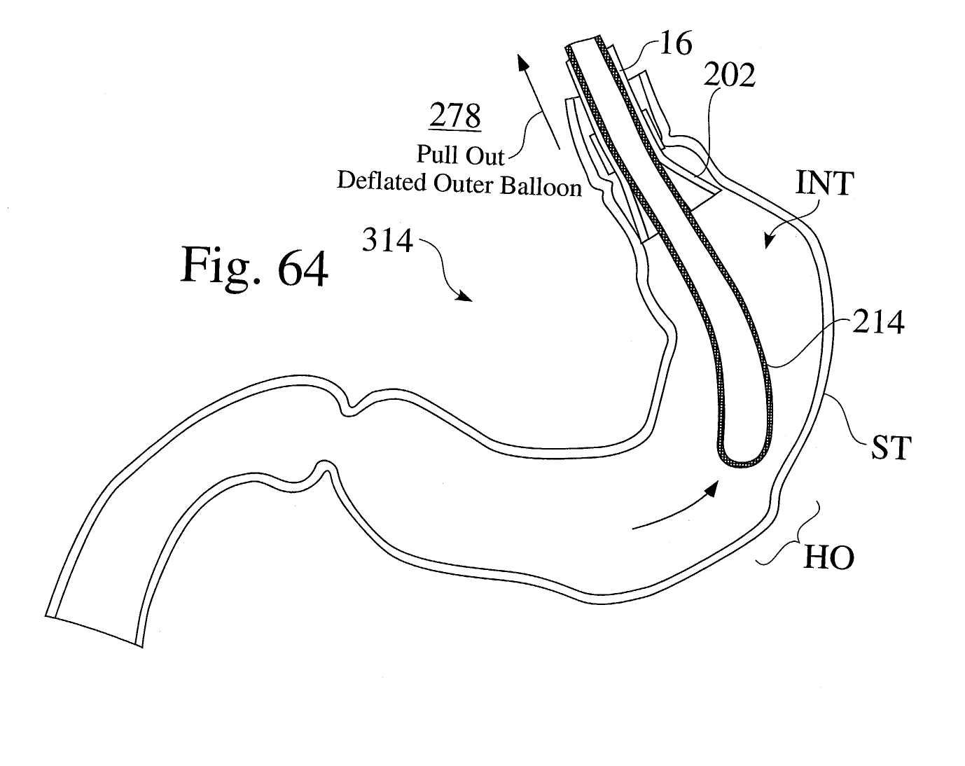

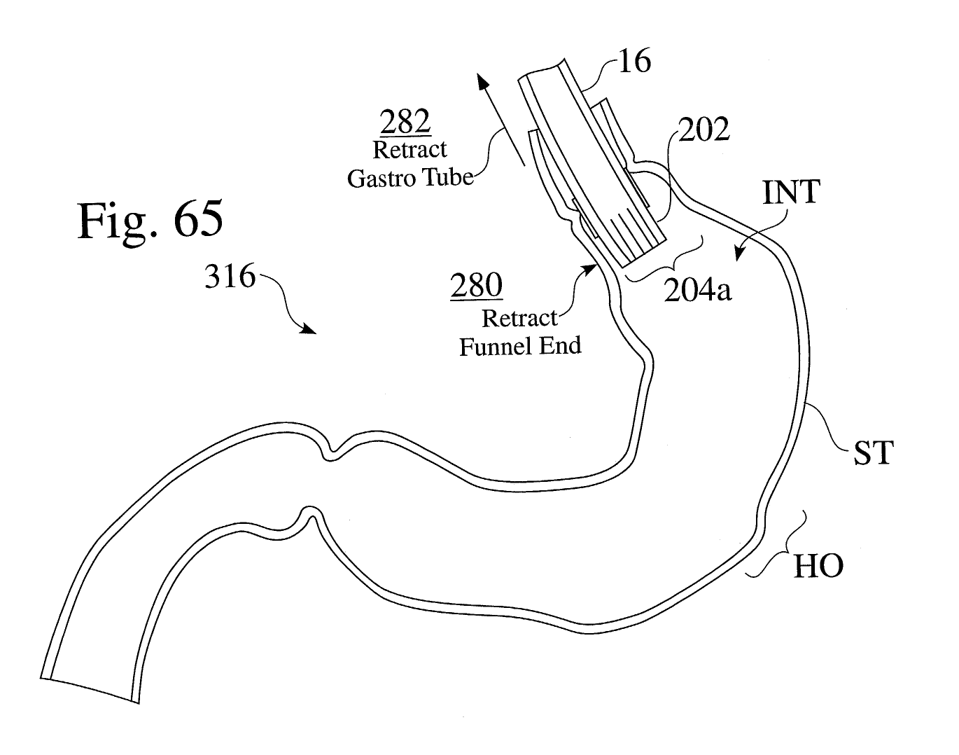

FIG. 63 is a cutaway view 312 which shows the saline removal and deflation 276 of the outer balloon 214 in the second embodiment of a staged balloon ablation process 250. FIG. 64 is a cutaway view 314 which shows the removal 278 of the deflated outer balloon 214 from the interior INT of the hollow organ HO in the second embodiment of a staged balloon ablation process 250. The expanded funnel end 202 of the introducer tube 16 provides a smooth transition region by which the outer balloon 214 is readily guided during removal 278. FIG. 65 is a cutaway view 316 which shows funnel-end retraction 280 and removal 282 of the introducer tube 16 in the second embodiment of a staged balloon ablation process 250.

Alternate Ablation Mechanisms. A compliant balloon 12 which provides surface ablation zones may alternately be provided, such as for hollow organs HO in which penetration into tissue TI is not required for the application of energy.

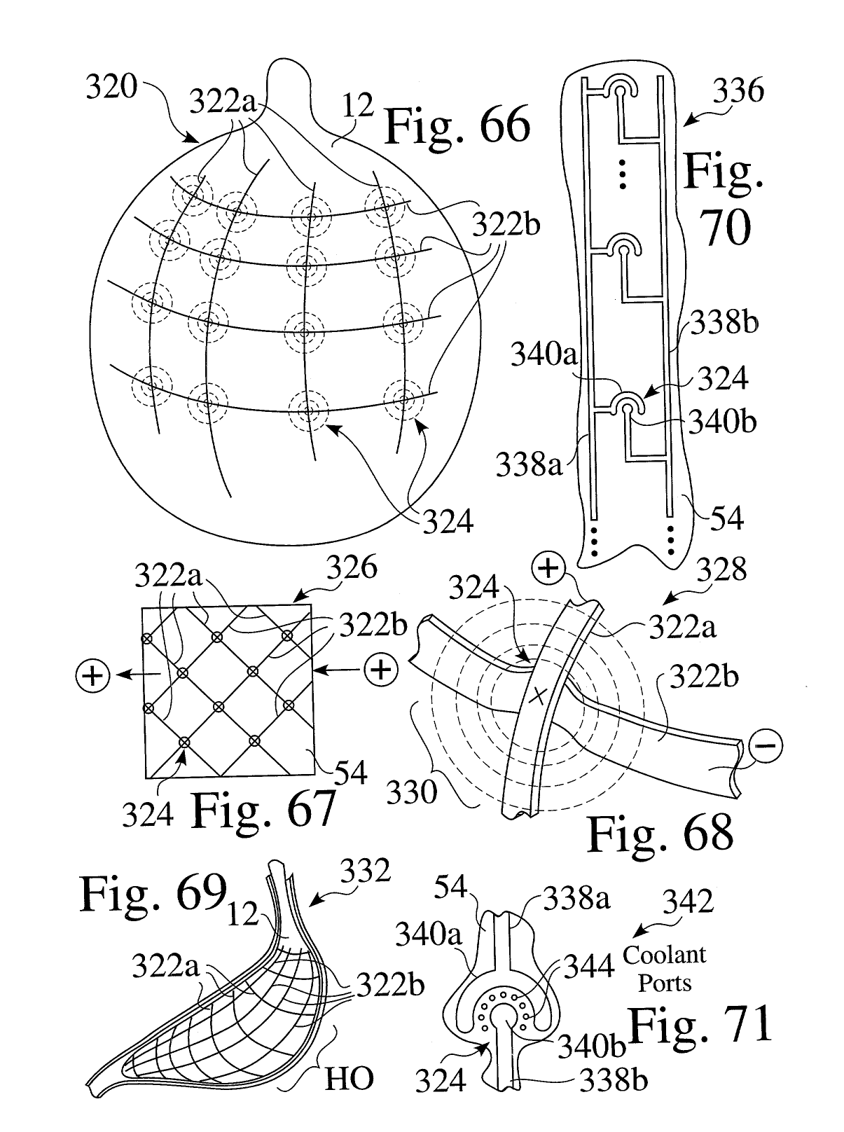

FIG. 66 is a partial perspective view 320 of bi-polar surface conductors 322a,322b for an ablation balloon 12, in which conductive traces 322a,322b are established on the balloon 12. FIG. 67 is a partial plan view 326 of conductive traces 322a,322b on a polymer substrate 54. FIG. 68 is a detailed partial perspective view of overlapping conductive traces and an ablation zone. FIG. 69 is a partial perspective view 332 of an ablation balloon 12 having overlaid bi-polar surface connections 322a,322b located within a stomach ST. The conductive traces 322 are typically comprised of an electrically conductive material, such as a carbon-filled polymer, or a metallic material which is patterned to expand with the complaint balloon 12. Ablation zones 324 are defined in intersecting regions between the sets of conductive traces 322a,322b. When energy 36, such as an RF energy potential 36, is applied across the intersecting regions 324, the regions 324 can be used to produce localized ablation 330, based on the applied energy level and the time of application.

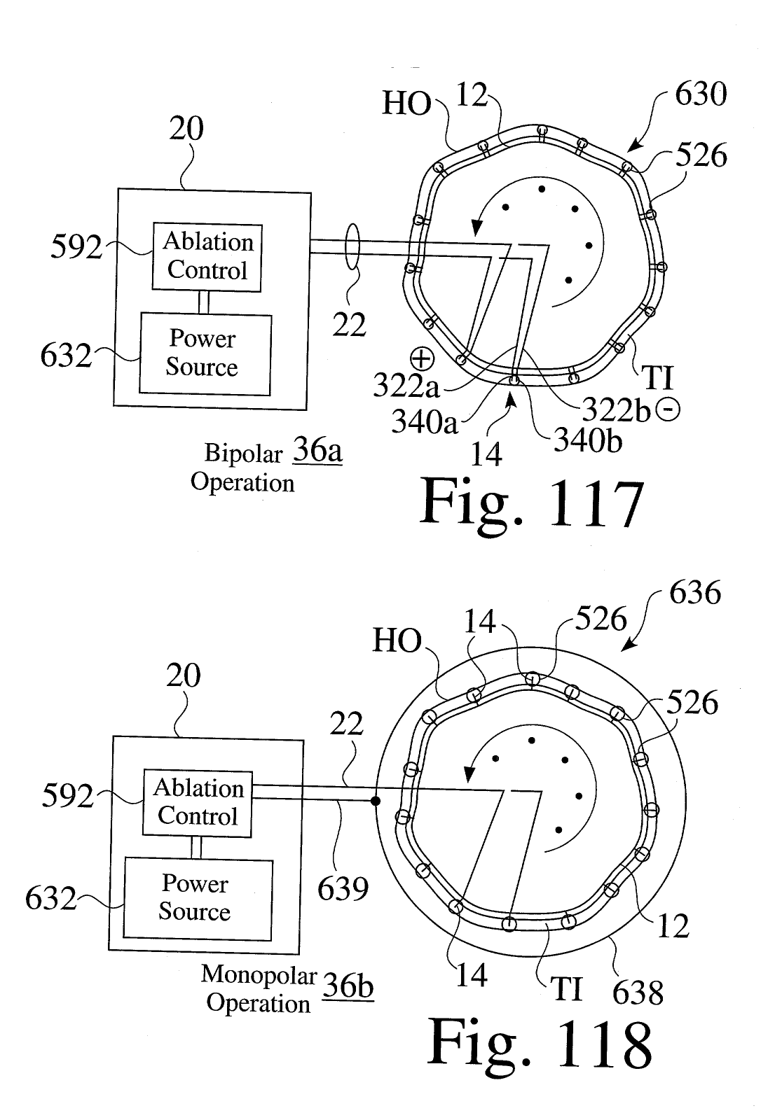

FIG. 70 is a schematic plan view 336 of an alternate embodiment for bi-polar surface conductors, in which conductors 338a, 338b are established on a substrate 54 which can be placed into contact with tissue TI. Probe electrodes 340a extend from the conductor 338a, while opposing probe electrodes 340b, in close proximity to the first probe electrodes 340a, extend from the second conductor 338b. The local regions between the opposing electrodes 340a,340b defines probe ablation zones 324 on the substrate 54, such as to locally apply energy 36 to a controlled region of a hollow organ HO. FIG. 71 is a detailed schematic plan view of bi-polar surface conductors 338a, 338b having coolant ports 344 with a defined ablation zone 324. As energy 36 may be controllably applied to the relatively small ablation zones 324. the use of coolant 148, such as a saline solution 148, can protect the tissue from local overheating during bipolar ablation 36a (FIG. 117).

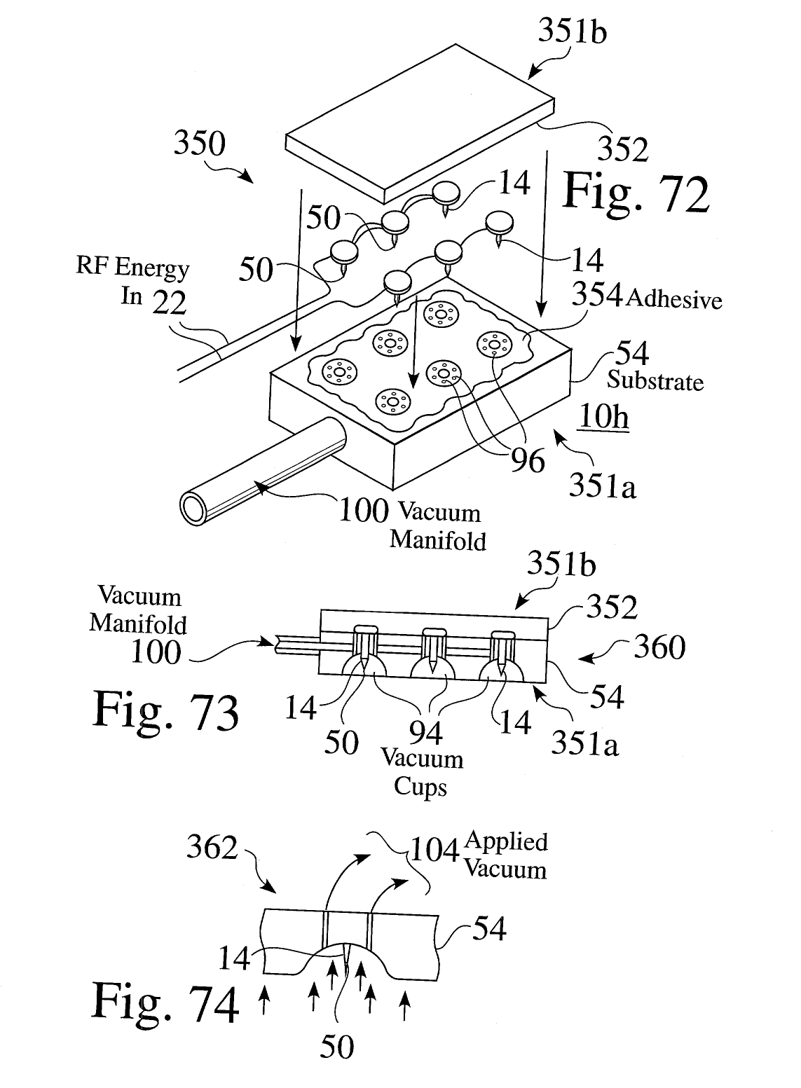

Alternate Ablation Systems. FIG. 72 is a perspective assembly view 350 of an alternate ablation apparatus 10h having vacuum deployment 100, which is typically deployed locally to tissue TI. FIG. 73 is a partial cross sectional view 360 of an ablation apparatus 10h. FIG. 74 is a detailed partial cross sectional view 362 of vacuum probe needle deployment for an ablation apparatus 10h. The ablation apparatus 10h includes probe needles 14 which extend into recess regions 94 on a probe face 351a. The probes 14 are fixedly positioned between a substrate 54 on the probe face 351a and a retainer 352 on the opposing face 351b. An adhesive 354 is typically used to affix the substrate 54 to the retaining layer 352. Vacuum ports 96 extend from the recess regions 94 to a vacuum manifold 100.

For applications in which the ablation apparatus 10h is deployed within a hollow organ HO, a secondary distension and/or positioning apparatus 431 (FIG. 81) may also be positioned within the hollow organ HO, to distend the hollow organ HO, and/or to correctly position the ablation apparatus 10h over a portion of tissue TI.

The ablation apparatus 10h is comprised of electrically conductive needle probes 14, having tips 50 which are located below the operational surface 351a of a substrate 54, within hollow cup regions 94. The ablation apparatus 10h includes one or more electrical connections 22 to each of the needles 14, for measurement or for the application of ablation energy. As well, the ablation apparatus 10h comprises a vacuum manifold 100 connected to the hollow cup regions 94. When the ablation apparatus 10h is positioned over tissue TI of a hollow organ HO, an applied vacuum 104 to the vacuum manifold 100 acts to draw the tissue TI into the cup regions 94, such that the tissue TI comes into contact with the needle probes 14.

The exemplary ablation apparatus 10h shown in FIG. 72 and FIG. 73 shows a layered construction, in which the electrically conductive needles are sandwiched between the substrate 54 and a rear cover 352, which is located on the back surface 351 of the ablation apparatus 10h. An adhesive 354 is typically used to bond the substrate 54 to the tear cover 352.

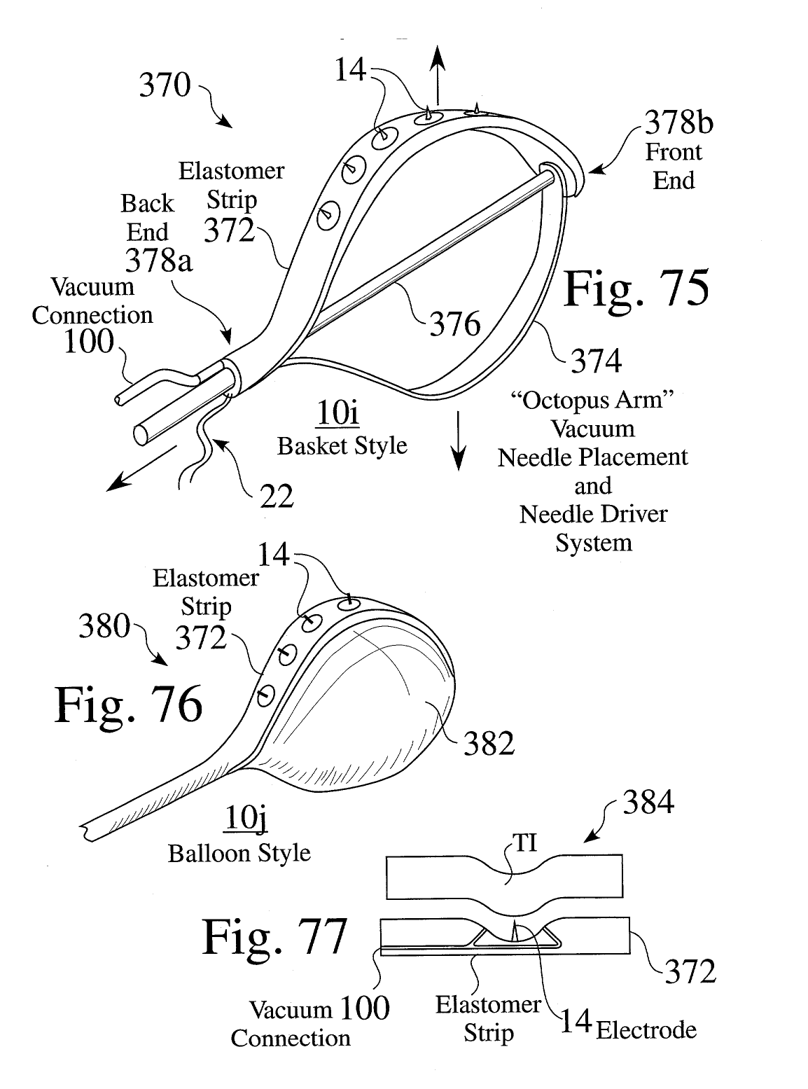

FIG. 75 is a perspective view 370 of an octopus basket arm ablation apparatus 10i having vacuum deployment. FIG. 76 is a perspective view 380 of a balloon arm ablation apparatus 10j having vacuum deployment. FIG. 77 is a detail view 384 of vacuum needle deployment for an octopus arm 372.

As seen in FIG. 75 and FIG. 76, a flexible octopus arm 372 is comprised of an elastomer strip and one or more deployable needles 14, having electrical connections 22. The elastomer strip 372 shown in FIG. 75 is relatively fixed between the front end 378b and the back end 378a, while the elastomer strip 372 shown in FIG. 76 forms a relatively open loop between the front end 378b and the back end 378a, as it conforms to inflation of the balloon 382.

One or more of the needle probe locations 14 may further comprise a thermal sensor, such as a thermocouple 458 (FIG. 85). The octopus arm 372 typically comprises a vacuum manifold 100 connected to hollow cup regions 94. When the ablation apparatus 10h is positioned over tissue TI of a hollow organ HO, an applied vacuum 104 to the vacuum manifold 100 acts to draw the tissue TI into the cup regions 94, such that the tissue TI comes into contact with the needle probes 14.

The octopus basket arm ablation apparatus 10i includes a deployer 376, such as a rod or cable 376, between a back end 378a and a slidably fixed front end 378b. The octopus basket arm ablation apparatus 10i also comprises one or more flexible basket arms 374, which are similarly anchored to the opposing ends of the flexible octopus arm 372. When the octopus basket arm ablation apparatus 10i is placed within a hollow organ HO, such as stomach ST, a pulling force on the deployer 376 creates a curved arch in the flexible octopus arm 372 and in the flexible basket arms 374, thereby expanding the ablation apparatus 10i while contacting and typically distending the hollow organ HO.

In operation, after the basket arm ablation apparatus 10i is expanded, the needles 14 are controllably brought into contact with the tissue TI of the hollow organ HO, such as by application of an applied vacuum 104 to the vacuum manifold 100. As described above, the needles 14 may preferably further comprise an insulating region 74 (FIG. 10, FIG. 11), such that the needles 14 do not electrically contact the mucosal layer MU of a hollow organ HO. When the ablation apparatus 10i is deployed, impedance measurement, application of energy, and monitoring is typically controlled by an attached processor and monitor unit 20 (FIG. 1).

The octopus basket arm ablation apparatus 10i is similarly removed from a hollow organ HO. After the probe needles 14 are returned to an undeployed position 44a, the deployer 376 is released or pushed to return the flexible octopus arm 372 and the flexible basket arms 374 to an unexpanded position. The ablation apparatus 10i is then removed from the hollow organ HO, such as by retraction through an introducer tube 16 (FIG. 32).

As seen in FIG. 76, the balloon arm ablation apparatus 10j is similarly comprised of a flexible octopus arm 372 having one or more deployable needles 14, having electrical connections 22. The balloon arm octopus arm ablation apparatus 10j includes a balloon 382, between a back end 378a and a front end 378b. When the balloon arm ablation apparatus 10j is placed within a hollow organ HO, such as stomach ST, inflation of the balloon 382, such through a pressure connection 24 from an applied pressure source 116, creates a curved arch in the flexible octopus arm 372, thereby expanding the ablation apparatus 10j, while contacting and typically distending the hollow organ HO. The needles 14 are then brought from an undeployed position 44a to a deployed position 44b, to controllably contact the tissue TI of the hollow organ HO.

Ablation System Having Inflatable Deployment. FIG. 78 is a perspective view 390 of an inflatable bladder needle driver ablation apparatus 10k. An inflatable bladder 392, having deployable electrically conductive probe needles 14, is located substantially within a channel shaped support structure 394. An external indeflator 398, comprising an inflator 400, is connected to the ablation apparatus 10k by connection 396. The inflator preferably includes a pressure monitor 402, such as a gauge or display 402. The apparatus also includes electrical connections 22, such as for impedance measurement 26, ablation energy 36, and/or temperature measurement. The electrical connections are preferably routed through the connector 396, by a junction 397, and typically include an adapter connector 404 for connection to a processor and monitor unit 20 (FIG. 1).

FIG. 79 is a partial perspective cutaway view 410 of an inflatable bladder 392 in a first undeployed position 412a, in which the probe needles 14 are located within the protective channel region 414. FIG. 80 is a partial perspective cutaway view 420 of an inflatable bladder 392 in a second deployed position 412b, in which the probe needles extend beyond the protective channel region 414.

FIG. 81 is a partial perspective view 430 of inflatable bladder needle driver ablation apparatus 10k located within a hollow organ HO, and further comprising a distending balloon 431. By placement of the channel 394 against the interior surface of a hollow organ HO, such as a stomach ST, the probe needles 14 may be controllably moved between an undeployed position 44a, in which the probe needles 14 do not contact the tissue TI, and a deployed position 44b, in which the probe needles 14 extend into the tissue TI, such as through a mucosal layer MU. The distending balloon 431 is controllably inflated to distend the hollow organ HO, such as to promote probe contact between the ablation apparatus 10k and the tissue TI.

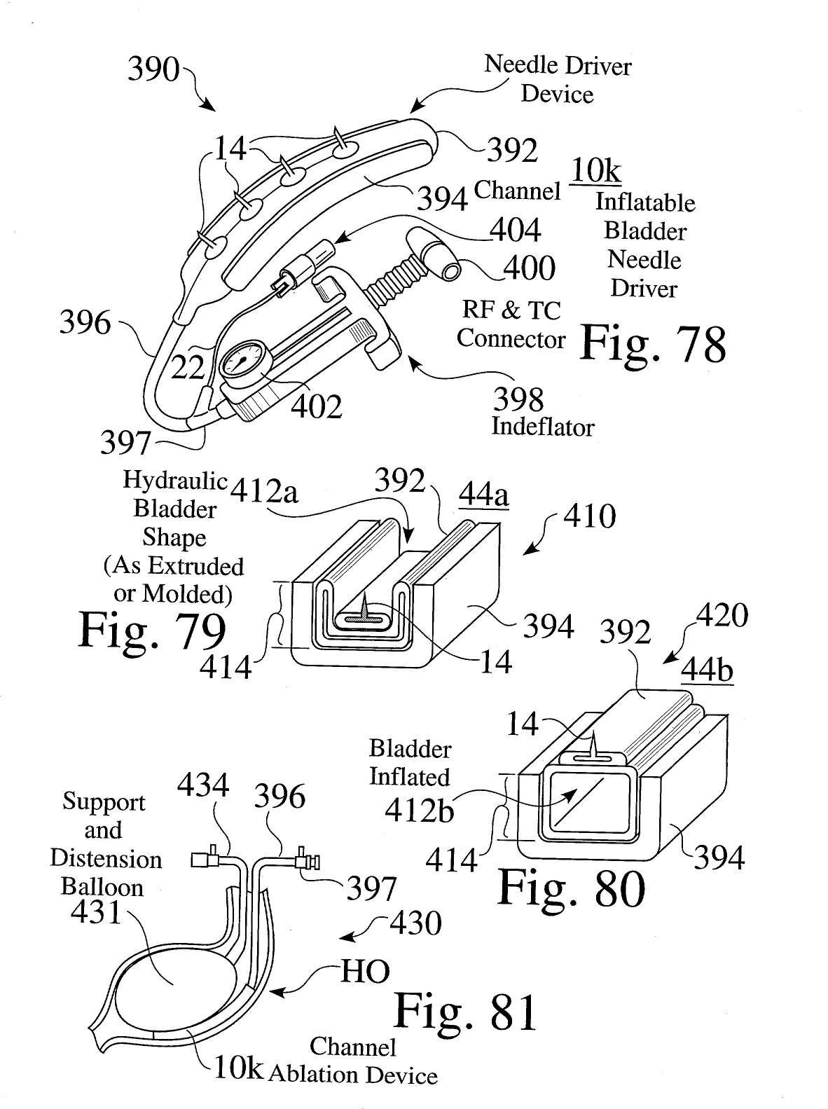

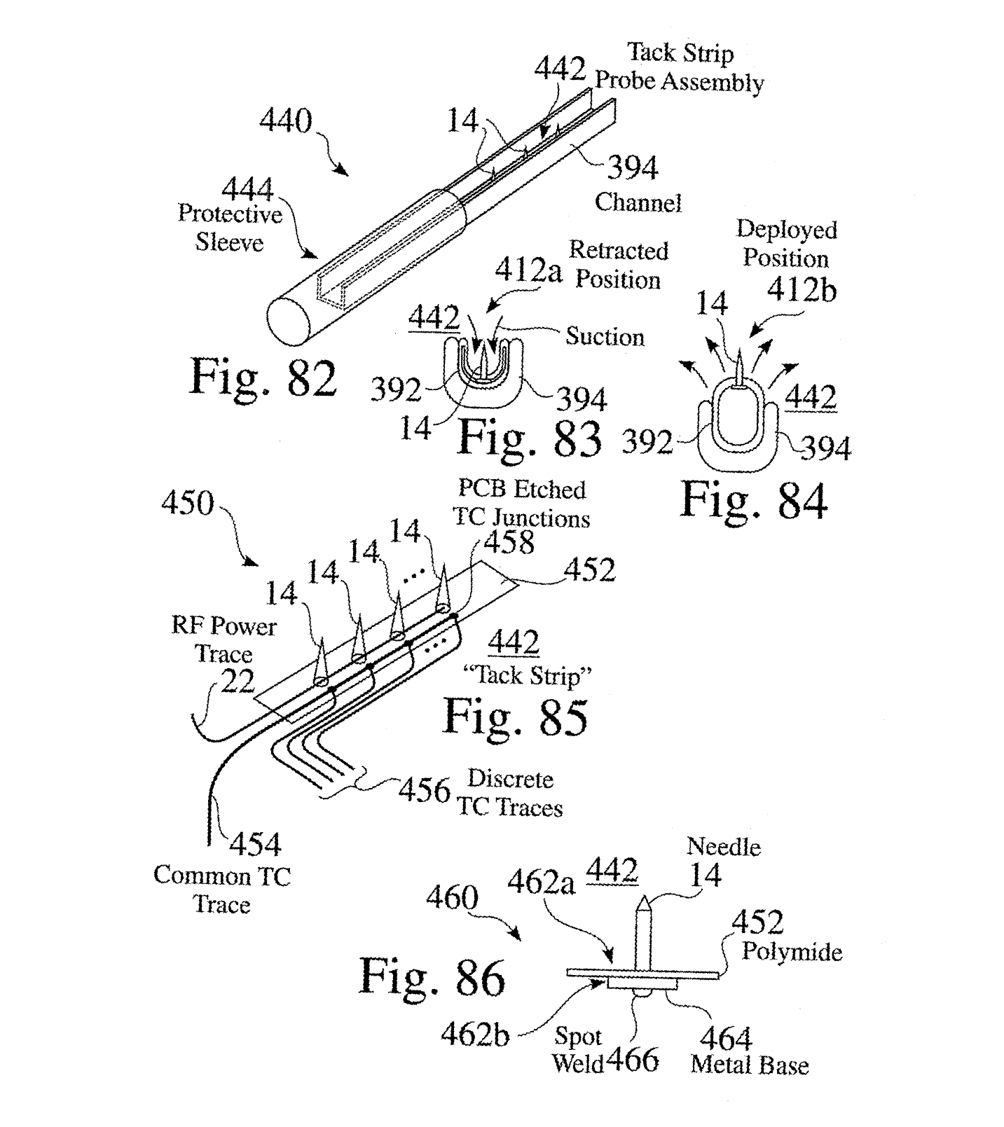

FIG. 82 is a perspective view 440 of a probe needle tack strip 442 and channel 394 which are slidably held and deployed by a protective sleeve 444. FIG. 83 is a partial cross sectional view of an RF needle tack strip having an inflatable bladder 392 in a first undeployed position 412a with a channel 394. FIG. 84 is a partial cross sectional view of an RF needle tack strip 442 having an inflatable bladder 392 in a second deployed position 412b extending from a channel 394.

Probe Needle and Sensor Mechanisms. Probe needles 14 can be fabricated either individually, or as a pre-fabricated structure or strip 442 comprising one or more probe needles 14. FIG. 85 is a perspective view 450 of an RF needle tack strip 442 having a plurality of probe needles 14 attached to a flex circuit 452. One or more electrical connections 22 are also established to the probe needles 14, such as by a common trace 22, or by discrete connections 22.

The tack strip 442 also preferably comprises an etched thermocouples 458, comprising one or more connections between thermocouple-pair metal traces 454,456, e.g. such as between copper-constantan type-T pairs 454,456, or between chromel-alumel type “K” pairs 454,456.

In various embodiments of the ablation systems 10, a wide variety of thermal sensors 458 may be used, such as but not limited to thermistors, RTDs, and thermocouples 458, and can be an integrally fabricated assembly, or may alternately be an attachable thermal sensor assembly 458. The thermal sensors 458 can be located within the needles 14, and can be located elsewhere within the assembly, such as within intimate thermal contact with the needles 14, or slightly thermally separated from the needles 14, such as to provide accurate temperature measurement for the surrounding ablated tissue.

FIG. 86 is a partial cross sectional view 460 of an RF needle tack strip 442 having a flex circuit 452, such as a polyimide substrate, and probe needles 14 which extend from the trace side 462a of the substrate 452. As seen in FIG. 86, the probe needles 14 are attached to a metal base 464 on the second side 462b of the substrate 452, by spot welds 466.

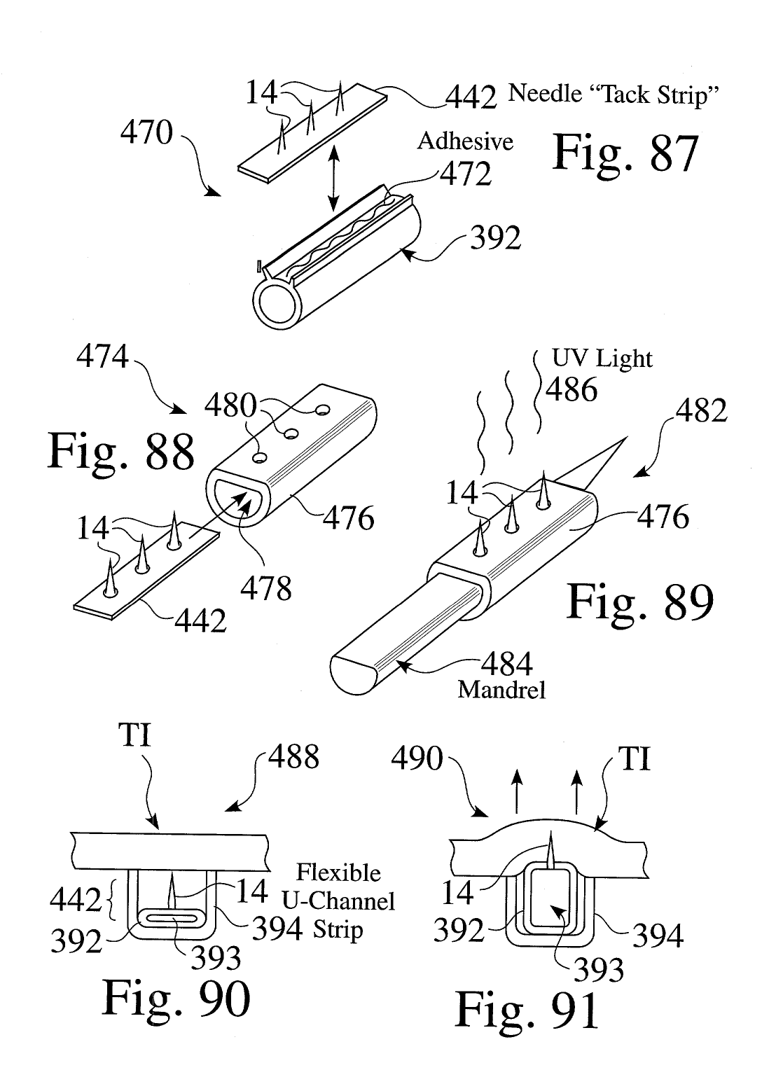

FIG. 87 is a perspective cutaway assembly view 470 of a needle driver apparatus having a one or more probe needles 14 on a tack strip 442, which is adhesively mounted 472 to the exterior of a hollow extrusion 392.

FIG. 88 is a perspective assembly view 474 of a mandrel needle driver apparatus having a one or more probe needles 14 on a tack strip 442. The tack strip 442 is mounted 472 within the interior 478 of a hollow extrusion 476, such that the probe needles 14 extend through holes 480 in the extrusion 476. FIG. 89 is a perspective view 482 of a mandrel needle driver apparatus, in which a mandrel 484 is located within the interior 478 of the hollow extrusion 476, which is typically comprised of a polymer, such as PVC or PET. The mandrel 476 fixedly holds the tack strip 442 in position. The hollow extrusion 476 may preferably be comprised of a UV or heat curable polymer, such that the hollow extrusion 476 shrinks to form a secure probe assembly.

FIG. 90 is a partial cross sectional view 488 of an RF needle tack strip 442 having an inflatable driver 392,393 in a first undeployed position within a channel 394. FIG. 91 is a partial cross sectional view 490 of an RF needle tack strip 442 having an inflatable driver 392,393 in a second deployed position within and extending from a channel 394, in which the probe needles 14 pierce and establish electrical contact with tissue TI.

Needle Tack Strips. FIG. 92 is a partial cross sectional view 492 of a hypotube ablation tack strip 442a, in which each probe needle 14 is comprised of a hypotube 494 having a hollow bore 496. The probe needles 14 are attached to a tack strip substrate 497 by a spot weld 498. FIG. 93 is a perspective view 500 of a hypo tube tack strip 442a. The tips 50 of the probe needles 14 are preferably cut at an angle across the hollow hypotube 494, to provide a sharp leading tip 50.

FIG. 94 is a perspective view 502 of a center punch-up tack strip 442b, in which one or more probe needles 14 are formed by punch areas 504a located within the inner region of an electrically conductive tack strip substrate 497. FIG. 95 is a perspective view 506 of a side punch-up tack strip 442c, in which one or more probe needles 14 are formed by punch areas 504b located along an edge of an electrically conductive tack strip substrate 497.

FIG. 96 is a perspective view 508 of a spot welded hypotube tack strip 442d, in which one or more hollow hypotubes 494 are flattened and spot-welded 510 to an electrically conductive tack strip substrate 497. FIG. 97 is a perspective view 512 of a spot welded flat needle tack strip 442e, in which one or more bent probe needles 14 are spot-welded 514 to an electrically conductive tack strip substrate 497.

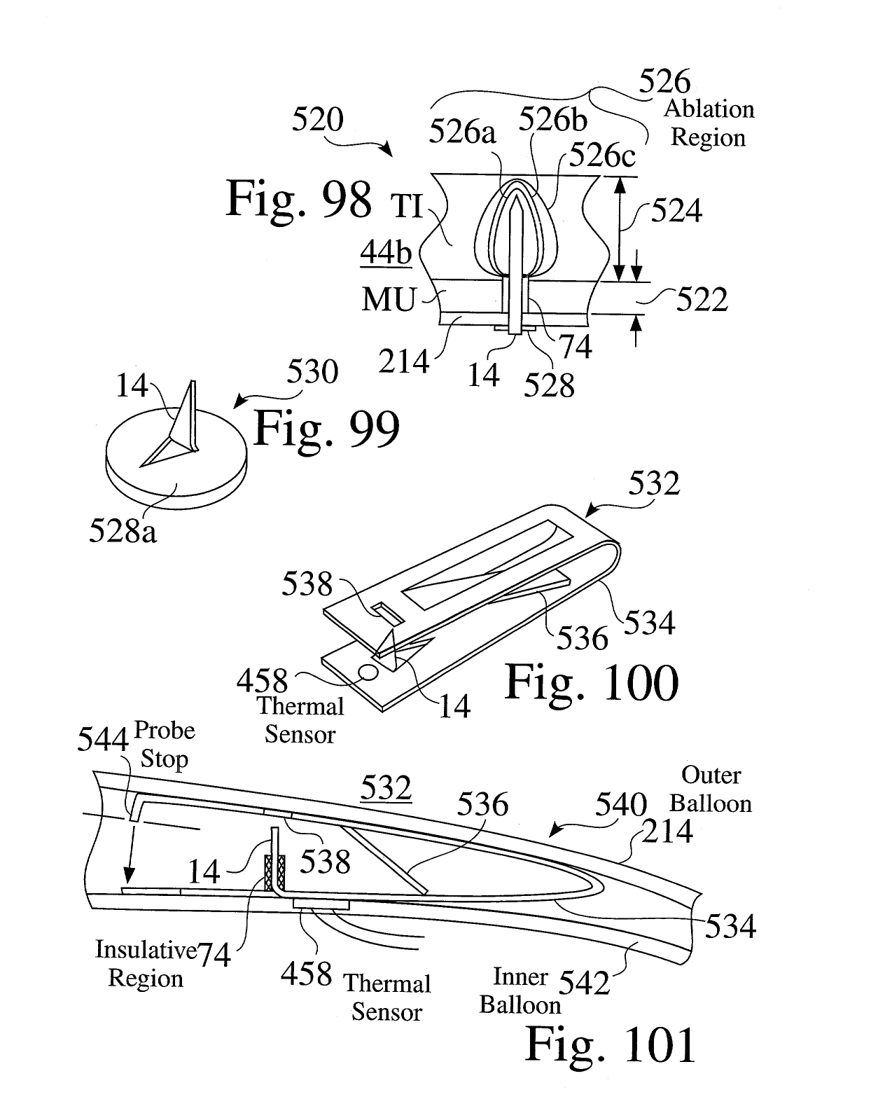

Tissue Ablation. In many of the embodiments of the ablation apparatus 10, the probe needles 14 act as a hypodermic “thumbtack”, to establish contact with the tissue TI of a hollow organ HO, and can be deployed by a wide variety of mechanisms and processes. FIG. 98 is a partial cutaway view 520 of ablation regions 526a,526b,526c established within the tissue TI of a hollow organ HO. As seen in FIG. 98, the probe needles 14 preferably comprise an insulative region 74, which provides electrical insulation between the probe needles 14 and the mycosal region MU of a hollow organ HO.

Before ablation energy 36 is applied to the tissue TI of a hollow organ HO, impedance/resistance data 26 is typically collected, whereby the applied ablation energy 36 may preferably be based upon the resistance and/or capacitance of the tissue TI.

As ablation energy 36, such as RF energy 36, is applied to the tissue TI, typically as a function of magnitude and time, the tissue TI surrounding the probe needles 14 is controllably ablated, with an increasing effective ablation region 526a, 526b,526c. The establishment of an ablation regions 526 results in a controlled cooking and eventual scarring of a portion of the tissue TI, which results in a controlled reduction in size of all or a portion of a hollow organ HO. As ablated tissue TI within the hollow organ HO starts to heal, the ablated tissue TI shrinks, and draws the surrounding tissue together, permanently. This controlled shrinkage can be used to reduce the overall size of the hollow organ HO, such as for shrinkage of a stomach ST. While different tissue TI within the hollow organ HO may shrink less or more in some ablation systems 10, the hollow organ HO is proportionally and controllably shrunken. The controlled shrinkage can alternately be used to ablate or shrink only a portion of a hollow organ HO, or to selectably ablate certain neural regions within a hollow organ HO.

Alternate Needle Diving Mechanisms. The driving force for probe needles 14 is typically hydraulic, pneumatic, or some form of a combined hydraulic/pneumatic system. FIG. 99 is a simplified perspective view of a formed needle probe assembly 530, in which a needle probe 14 is formed from a base section 528a.

FIG. 100 is a perspective view of an integrated spring needle probe assembly 532. A needle probe 14 is formed on a leaf spring base 534, which is typically comprised of a flexible metal, such as a surgical quality spring steel or stainless steel. Needle probes 14 may also preferably comprise an external plating layer, such as to provide an inert protective layer, or to improve electrical conductivity.

FIG. 101 is a partial cutaway view 540 of an integrated spring needle probe 532 located between an inner activation balloon and 542 an outer distension balloon 214, in an undeployed position 44a. The leaf spring base 534 shown in FIG. 100 and FIG. 101 also includes a spring tab 536, which adds a bias force to the assembly 532, during deployment 44b. The assembly 532 also includes needle access hole 538. A probe stop 544 provides controlled travel limit for the needle probe 14, whereby the needle probe 14 is deployable to a controlled depth into tissue TI of a hollow organ HO, thereby defining a penetration depth, and reducing the possibility of tissue perforation. As seen in FIG. 101, the integrated spring needle probe assembly 532 preferably includes an insulative region 74, providing isolation between the needle probe 14 and the mycosal region of a hollow organ HO. FIG. 102 is a detailed partial perspective view 550 of an integrated spring needle probe spring base 534, having a thermal sensor mounting region 552. FIG. 103 is a detailed partial perspective view 554 of an alternate integrated spring needle probe spring base 534, having an integrated conductor trace 556.

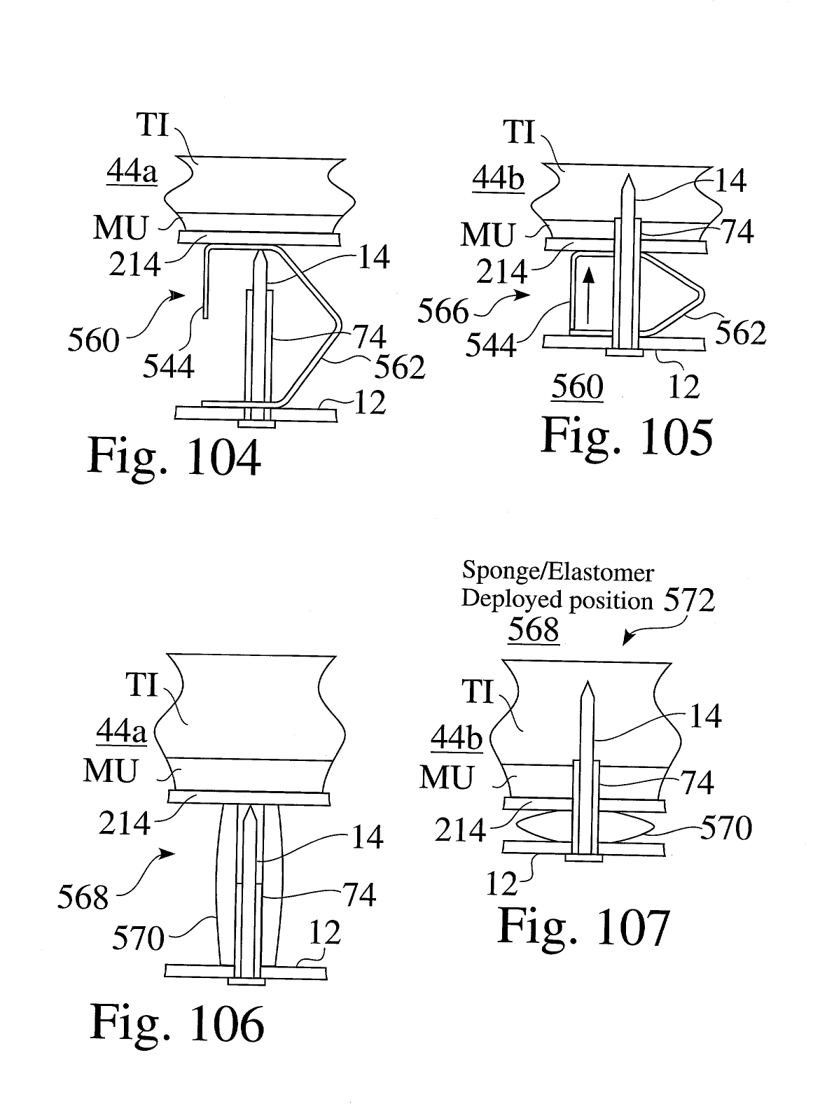

FIG. 104 is a partial cutaway view of a leaf spring needle probe assembly 560 in an undeployed position 44a. FIG. 105 is a partial cutaway view 566 of a leaf spring needle probe 560 in a deployed position 44b. The leaf spring 562 can be formed in a variety of shapes, such as to include a travel stop 544.

FIG. 106 is a partial cutaway view of a polymer spring needle probe assembly 568 in an undeployed position 44a. FIG. 107 is a partial cutaway view of a polymer spring needle probe 568 in a deployed position 44b. The polymer spring 570 is preferably comprised of an elastomer, such as a compliant solid elastomer, or a closed-cell or open-cell foam. While the polymer spring 570 is shown generally as a compressible cylinder, the polymer spring 570 can be formed in a wide variety of shapes, and the assembly can also comprise a depth control limit 544, either as an integrated detail of the spring 570, or as a separate assembly component.

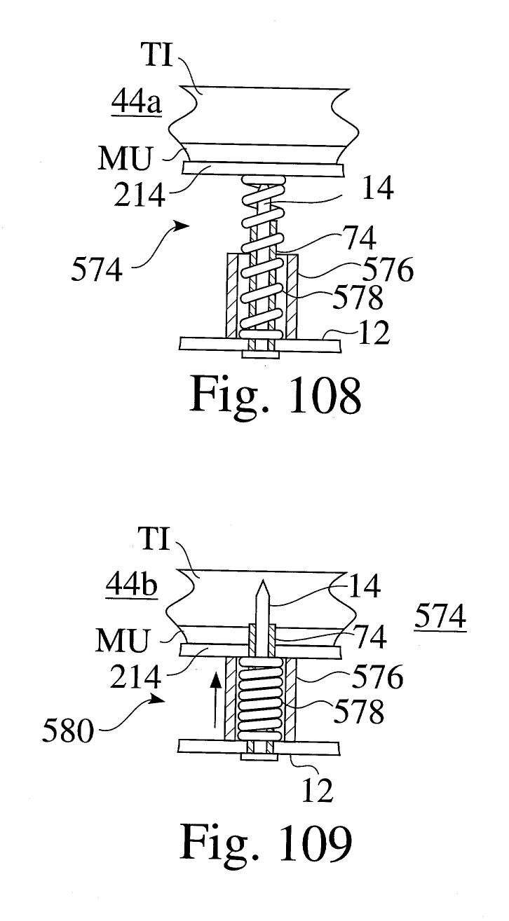

FIG. 108 is a partial cutaway view of a coil spring needle probe assembly 574 in 1 an undeployed position 44a. FIG. 109 is a partial cutaway view 580 of a coil spring needle probe 574 in a deployed position 44b. The coil spring needle probe assembly 574 comprise a depth control limit 576, either as an integrated detail of the spring 570, or as a separate assembly component.

FIG. 109 shows a mycosal layer MU of approximately 1 mm, with a stomach wall tissue of approximately 2-3 mm. As seen in FIG. 109, when a probe needle assembly is in a deployed position 44b, the probe needles 14 extend through the mycosal layer MU and beyond, into the tissue TI of a hollow organ HO, such as into a stomach wall. It is preferable to protect the mycosal layer MU of a stomach ST, such that the mycosal layer MU is not overheated during a ablation steps 36. For example, ablation may be controlled as a function of temperature and time, e.g. such as a controlled temperature of 50 to 75° C., for intervals of 5 to 15 minutes. As well, as described above, a portion of the needle probes 14 may preferably comprise an insulative section 74, typically comprised of an electrically insulative material, such as polyimide, nylon, or polyester, to prevent the localized overheating of a mycosal layer MU.

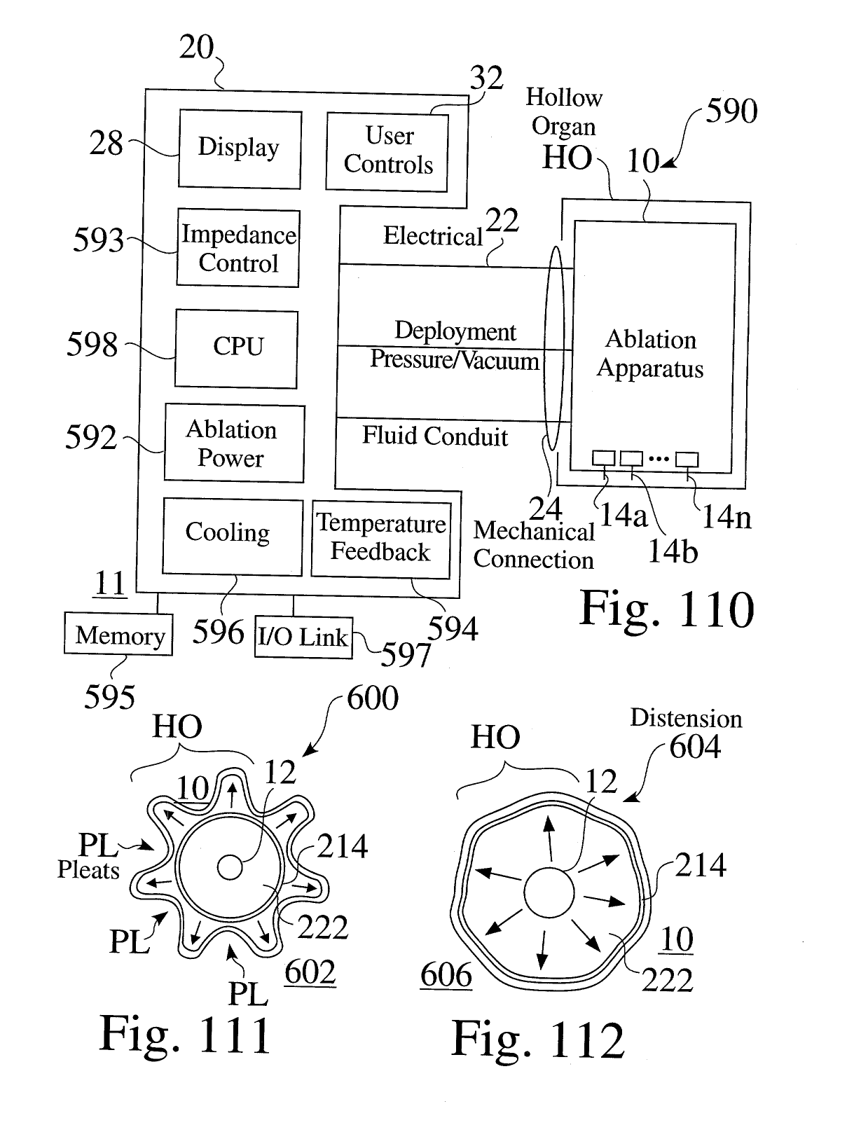

System Block Diagram. FIG. 110 is a simplified functional block diagram 590 of the deployable ablation system 11, in which an ablation apparatus 10, having one or more deployable needle probes 14a-14n, is controllably positioned within a hollow organ HO. The ablation apparatus 10 is connected to an external monitoring and processing unit 20, by electrical connections 22 and mechanical connections 24, such as pressure, vacuum, and/or process fluid connections, as described above.

The external monitoring and processing unit 20 shown in FIG. 110 includes impedance control 593, ablation power 592, temperature feedback 594, cooling 596, and central processing unit CPU 598, as well as a user interface 32 and display 28. As well, the external monitoring and processing unit 20 may further comprise memory storage 595 for acquired data and/or to record applied energy 36, and may include an I/O link 597, such as to connect the external monitoring and processing unit 20 to a printer, to a computer, or to a network.

The cooling system 596 is preferably used in some embodiments of the selective ablation system 11, such as to provide a larger ablation region 526 in the tissue TI around the needle probes 14, without localized overheating of the tissue TI or mycosal layer MU. As well, the cooling system 596 can protect the ablation apparatus 10, e.g. such as a probe balloon 12, from local overheating during the application of ablation energy 36.

For some embodiments of the selective ablation system 11 having process fluid delivery, such as saline 148 for cooling and/or electrical conduction, the external monitoring and processing unit 20 preferably includes or is compatible with other fluid delivery systems, such as for the controlled delivery of pharmaceutical solutions.

While the current embodiments are described as using RF powered ablation, e.g. such as 650 MHz), alternative ablation systems may use a variety of energy sources, such as microwave, laser, and/or radiant heat. The external monitoring and processing unit 20 typically controls the application of energy 36, based upon the desired magnitude and location of ablation 36 within the hollow organ HO. The ablation power 592 is typically controllable, based upon parameters such as but not limited to control data 26, desired ablation temperature, time of application of energy 36, and the location of probes 14.

In some embodiments of the external monitoring and processing unit 20, the frequency of the ablation power 592 is variable. In alternate embodiments of the external monitoring and processing unit 20, the power module 592 comprises a plurality of energy sources, such as to provide different energy 36 to any or all regions of a hollow organ HO in an integrated procedure, e.g. such as the application of ablation energy 36 for tissue shrinkage, as well as the application of the same or different energy 36 for identified focal nerve sites.

Hollow Organ Distension and Ablation System Positioning. FIG. 111 is a partial cutaway view 600 of an expandable ablation device 10 within a hollow organ HO, such as a stomach ST. Hollow organs HO typically comprise a large number of pleats PL, while in a natural non-distended position 602. The selective ablation system 10 is therefore preferably expandable, such as through the use of an outer compliant balloon 214 and a compliant probe balloon 12, whereby the hollow organ HO can be distended. FIG. 112 is a partial cutaway view 604 of an expanded outer balloon 214, which extends a pleated hollow organ HO to an distended position 606, in which the outer balloon 214 substantially contacts a large portion of the interior surface are of the hollow organ HO, including the pleated regions PL.

As seen in FIG. 111 and FIG. 112, a compliant probe balloon 12 is located within the interior region 222 (FIG. 36) of the outer balloon 214. The compliant probe balloon 12 is then inflated, as described above, such as by the introduction of a gas or a process fluid 148, e.g. saline, to substantially conform to the inflated outer balloon 214 and to the distended hollow organ HO.

Once the compliant probe balloon 12 is expanded to substantially conform to the inflated outer balloon 214, the needle probes 14, which populate any portion of the surface of the probe balloon 12, are deployed 44b to contact the tissue TI of the hollow organ HO. In some embodiments of the expandable ablation device 10, the compliant probe balloon 12 is more compliant than the inflated compliant outer balloon 214, such that the probe balloon 12 initially conforms to the interior 222 of the inflated outer balloon 214, and upon deployment of the probes 14 to a deployed position 44b, the probes extend through the surface of the inflated compliant outer balloon 214, rather than causing further distension of the inflated compliant outer balloon 214.

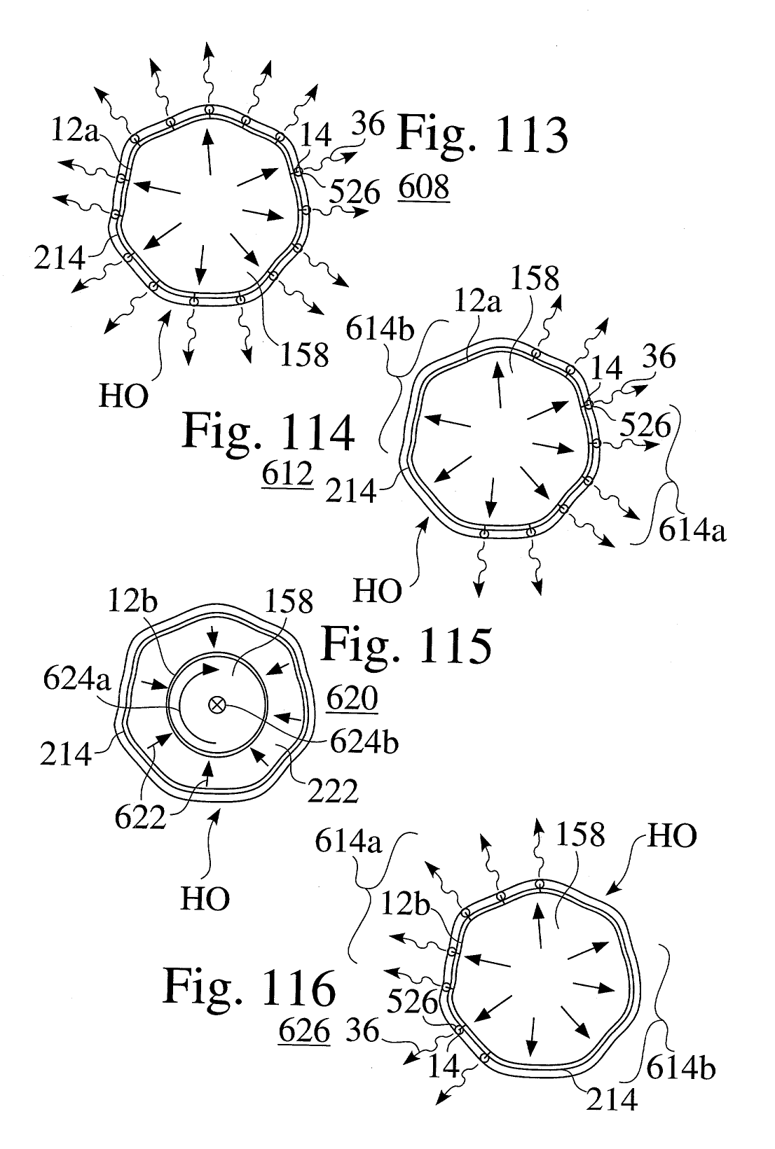

FIG. 113 is a partial cutaway view 608 of an expanded probe balloon 12a, having ablation energy 36 applied to probe needles 14 which are located across the entire perimeter of a distended pleated hollow organ HO. As described above, some embodiments of the selective ablation system 10 provide substantial needle probe coverage, whereby ablation 36 can be controllably performed in a single probe balloon position, as seen in FIG. 113.

FIG. 114 is a partial cutaway view 612 of selective ablation 36 over a portion of a distended pleated hollow organ HO. Alternate embodiments of the compliant probe balloon 12b include probe needles 14 on a portion 614a of the perimeter of the probe balloon 12b, while other portions 614b do not include needle probes 14. In some embodiments of the selective ablation system, a compliant probe balloon 12b is used for selective reshaping of a hollow organ HO, such as to reduce the surface area of a specific interior region of a hollow organ HO.

In other embodiments of the selective ablation system 10, a compliant probe balloon 12b is repositioned one or more times, such as to acquire impedance data 26 or to apply ablation energy 36 to different areas of a hollow organ HO. FIG. 115 is a partial cutaway view 620 showing the partial deflation 622 and rotation 624a of a compliant probe balloon 12b within distended pleated hollow organ HO. The outer balloon 214 is typically retained in an expanded position, whereby the deflated probe balloon 12 is readily rotationally positioned 624a and/or axially repositioned 624b within the interior of the hollow organ HO. Saline solution 148 can also be introduced within the interior region 222 of the outer balloon 214, such as for cooling, electrical conduction, and/or to reduce friction between the probe balloon and the out balloons during repositioning 624.

FIG. 116 is a partial cutaway view 626 of selective ablation 36 over a portion of a distended pleated hollow organ HO from a repositioned compliant probe balloon 12b.

System Configurations. Embodiments of the selective ablation system 11 can be configured for both bipolar ablation 36a and/or monopolar ablation 36b. FIG. 117 is a functional block diagram 630 showing bipolar ablation 36a within a hollow organ HO. Some embodiments of the selective ablation system 10 include probe regions 14 comprising locally opposing electrodes 340a,340b (FIG. 66-FIG. 71), creating localized ablation regions 526 between electrode paths 322a,322b. Coolant 148, such as saline 148, is commonly provided, through coolant ports 344 (FIG. 71) or needle coolant ports 150 (FIG. 26), to prevent local overheating during bipolar ablation 35a. As described above, some embodiments of the selective ablation system 10 include at least one opposing electrode 322, e.g. 322a, which comprises a deployable needle probe 14, which is deployable 44b to establish direct contact with a hollow organ HO. In alternate embodiments of the selective ablation system 10, the opposing electrodes 340a,340b are located on the surface of the probe balloon 12.

FIG. 118 is a functional block diagram 636 showing monopolar ablation 36b within a hollow organ HO. Some embodiments of the selective ablation system 11 include an electrical path 22 to deployable electrodes 14 on an ablation apparatus 10 which is positioned within a hollow organ HO, as well as an external connection 639 to one or more external band or patch electrodes 638. The band or patch electrodes 638 are typically placed outside the body of the patient PT, such as generally surrounding the region surrounding the location of the hollow organ HO to be mapped 26 and/or ablated 36. In alternate embodiments of the selective ablation system 11, the band or patch electrodes 638 are placed inside the body of the patient PT, surrounding the hollow organ HO to be mapped 26 and/or ablated 36.

The use band or patch electrodes 638 exterior to the hollow organ creates a generally distributed ablation region 526 surrounding the probe needles 14 during monopolar ablation 36b. While coolant 148, such as saline 148, may also be provided in a monopolar ablation system 10, such as through coolant ports 344 (FIG. 71) or needle coolant ports 150 (FIG. 26), monopolar ablation 36b typically provides less localized heating than bipolar ablation 36a.

Probe Groups. As described above, the deployable probe needles 14 can be selectably used, either individually or as a group, for any of the system operations, e.g. such as for impedance measurement 26, for the application of ablation energy 36, and/or for temperature measurement. It is preferable in several embodiments of the selective ablation system 10 to provide a large number of needle probes 14, to provide simple and rapid impedance measurement 26 and ablation 36, i.e. mapping and zapping, procedures. In some embodiments of the selective ablation system 10, the probe needles 14 are selectively addressed for data and diagnosis 26, while ablation energy 36 is controllably applied to all the probe needles 14 at the same time.

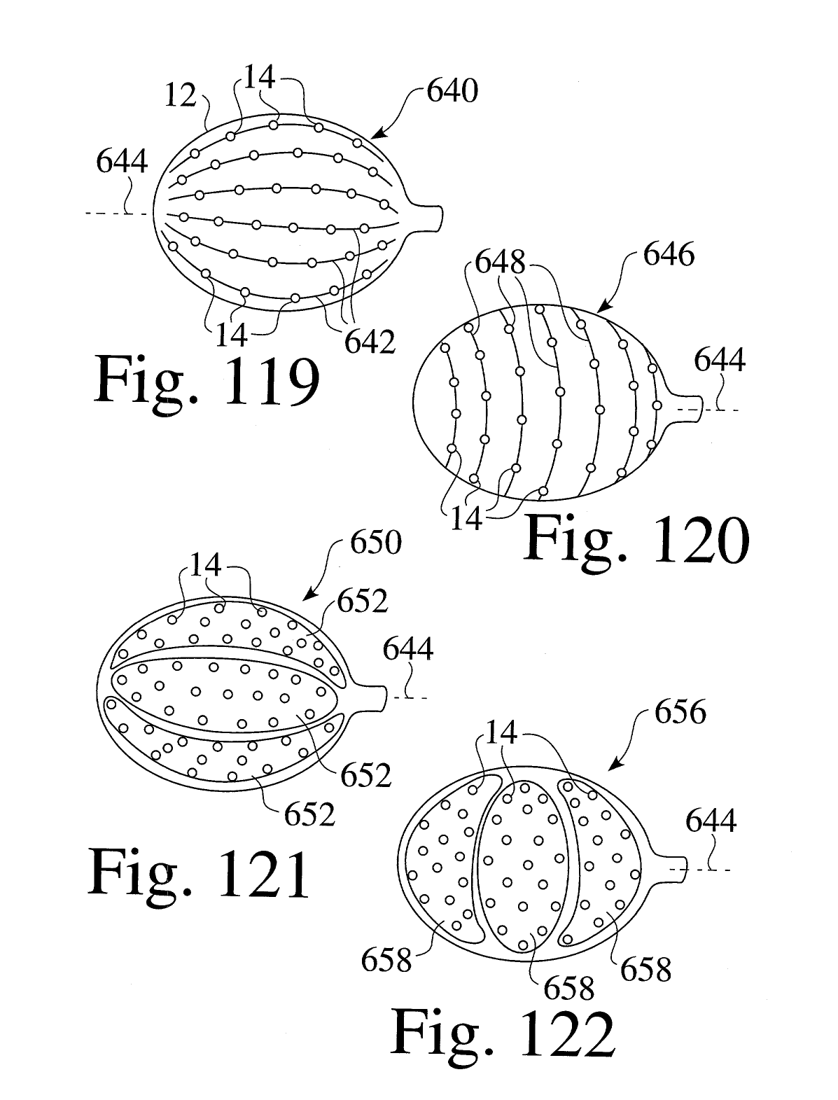

FIG. 119 is a side view 640 of a compliant probe balloon 12, generally aligned along a balloon axis 644, having one or more needle probes 14 arranged and electrically connected in axial, i.e. longitudinal, probe groups 642. FIG. 120 is a side view 646 of a compliant probe balloon 12, generally aligned with a balloon axis 644, having one or more needle probes 14 arranged and electrically connected in meridian, i.e. latitudinal, probe groups 648. FIG. 121 is a side view 650 of a compliant probe balloon 12, generally aligned along a balloon axis 644, having one or more needle probes 14 arranged and electrically connected longitudinal quadrant probe groups 652. FIG. 122 is a side view 656 of a compliant probe balloon, generally aligned along a balloon axis 644, having one or more needle probes 14 arranged and electrically connected in latitudinal quadrant probe groups 658.

While a probe balloon 12 may typically comprise a large number of needle locations 14, e.g. such as 50 to 70 needles 14, not all needle locations 14 are typically required to include temperature measurement devices 458. Temperature sensors 458, located at the one or more discrete locations in thermal contact with the needle probes 14, are typically used as representative locations for temperature measurement and monitoring. The temperature sensors 458 provide a temperature map for the probe balloon 12, which is collected by the central monitor and control unit 20, in which the temperature data is preferably used to monitor and control ablation 36. The central monitor and control unit 20 uses the temperature data to estimate a statistical temperature map for the ablation system and the hollow organ HO, with the estimated temperature range plotted over the local ablation zones 526, the surface area of the hollow organ, and/or the surface area of the ablation device 10.

Ablation Mechanism Testing. Testing of ablation mechanisms was performed on three Yucatan pigs on Nov. 27, 2001. A deployable electrode array 442, comprising a plurality of 3.5 mm needles 14, was used to deliver high density RF lesions across the outer surface of the stomach ST, covering antral, pyloric, and corporal regions. While ablation can be applied to either the inner surface of the outer surface of a hollow organ HO, such as a stomach, the application of energy to the outer surface during testing was readily achieved.

Pressure-volume curves of the stomach ST of each pig were measured prior before and after surgery. During the measurement of the pressure-volume curves, the abdomen was closed in the first pig, while the abdomens were open for the second and third pigs. A barostat was used to establish the measured pressure against an inflated balloon, before and after surgery.

Identical areas were treated in each of the pigs. In the first pig (Pig 1), a deployable electrode array 442 having a large number of deployable needles 14 was used to deliver high density RF lesions across the outer surface of the stomach ST, using several power settings and device parameters, over a period of approximately 4-5 hours. While the deployable electrode array 442 produced ablation areas in Pig 1, irregular lesions were produced. Removal of half of the electrodes appeared to improve the distribution of lesions. Table 1 provides ablation procedure data for Pig 1.

TABLE 4
Delivered Data - 3.5 mm Device - Pig 1

| | | Temp | | Set | | | |
|---|---|---|---|---|---|---|---|
| | Time | Set | Temp | Watt | | Dlvrd | Needle |
| Step | (min) | (° C.) | (° C.) | (W) | Ω | Watt | Density |
| 1 | 0 | 70 | 37 max | 50 | 110 | 10 | 100% |
| 2 | 1 | 70 | 37 max | 60 | 125 | 15 | 100% |
| 3 | 3 | 70 | 38->55 | 40 | 101-> | 40 | 100% |
| 4 | 5 | 70 | 55 | 42 | 79 | 42 | 100% |
| 5 | 4 | 70 | 53 | 42-45 | 85 | 45 | 100% |
| 6 | 4 | 70 | 41 | 42-45 | 87->75 | 45 | 100% |
| 7 | 3.4 | 70 | 36->55 | 45 | 78 | 45 | 100% |
| 8 | 2.9 | ″ | 41 | 45 | 78 | 45 | 100% |
| 9 | 1 | ″ | 41 | 45 | 78 | 45 | 100% |
| 10 | 4 | ″ | 71 | 60 | 70 | 60 | 100% |
| 11 | 2.6 | wet | 65 | 50 | 79 | 50 | 100% |
| | | with | | | | | |
| 12 | 8 | saline | 43 | 35 | 70 | 45 | 100% |
| 13 | 4 | turn | 51 | 25 | 70 | 50 | 50% |
| 14 | 4 | needle | 52 | 30 | 70 | 50 | 50% |
| 15 | 5 | up | 70 | 55 | 70 | 55 | 50% |
| 16 | 4.5 | ″ | 71 | 60 | 70 | 60 | 50% |
| 17 | 2.8 | ″ | 70 | 120 | 70 | 70 | 50% |
| 18 | 1.7 | ″ | 70 | 120 | 71 | 70 | 50% |
| 19 | 2 | ″ | 70 | 120 | 70 | 70 | 50% |
| 20 | 4 | 65 | 70 | 120 | 60 | 70 | 50% |
| 21 | 2 | 60 | 40 | 120 | 60 | 70 | 50% |
| 22 | 1.8 | 60 | 60 | 20 | 60 | 70 | 50% |

In the second pig (Pig 2), a deployable electrode array 442 having the reduced number of deployable 3.5 mm needles 442 was used to deliver high density RF lesions over the outer surface of the stomach ST, over a period of approximately 2 hours. When the set target temperature was reached, e.g. typically set at 80 C, the power was terminated Table 2 shows ablation procedure data for Pig 2.

TABLE 2
Delivered Ablation Data- 3.5 mm Device - Pig 2

| | | Temp | | Set | | | |
|---|---|---|---|---|---|---|---|
| | Time | Set | Temp | Watt | | Dlvrd | Needle |
| Step | (min) | (° C.) | (° C.) | (W) | Ω | Watt | Density |
| 1 | 1.6 | 60 | 42 | 120 | 100 | 70 | 50% |
| 2 | 3.6 | 60 | 60 | 120 | 73 | 60 | 50% |
| 3 | 3.2 | 60 | 60 | 120 | 74 | 60 | 50% |
| 4 | 2.8 | 60 | 60 | 120 | 72 | 60 | 50% |
| 5 | 1 | 70 | 70 | 120 | 70 | 60 | 50% |
| 6 | 1.5 | 70 | 73 | 120 | 70 | 60 | 50% |
| 7 | 1.5 | 70 | 70 | 120 | 70 | 60 | |
| 8 | 1.6 | 70 | 70 | 120 | 70 | 60 | |
| 9 | 2 | 70 | 70 | 120 | 66 | 60 | |
| 10 | 2 | 70 | 70 | 120 | 65 | 60 | |
| 11 | 2 | 70 | 70 | 120 | 68 | 60 | |
| 12 | 1.3 | | 70 | 80 | 80 | — | 30% |
| 13 | 0.7 | — | — | | 80 | 80 | 30% |
| 14 | 2 | 70 | 70 | 120 | 70 | 60 | |
| 15 | 3 | 70 | 72 | 120 | 70 | 60 | |
| 16 | 2 | 80 | 69 | 120 | 70 | 60 | |
| 17 | 2 | 80 | 80 | 120 | 70 | 60 | 30% |
| 18 | 2 | 80 | 82 | 120 | 70 | 60 | 30% |
| 19 | 2.5 | 80 | 86 | 120 | 70 | 60 | |
| 20 | 2 | 80 | 80 | 120 | 70 | 60 | |
| 21 | 2 | 80 | 80 | 120 | 70 | 60 | |
| 22 | 2 | 80 | 80 | 120 | 70 | 60 | |
| 23 | 1.5 | 80 | 80 | 120 | 70 | 60 | |
| 24 | 1.05 | 80 | 80 | 120 | 70 | 60 | |
| 25 | 2 | 80 | 80 | 120 | 70 | 60 | |
| 26 | 2.5 | 80 | 80 | 120 | 70 | 60 | |
| 27 | 2.5 | 80 | 80 | 120 | 70 | 60 | 30% |
| 28 | 2.5 | 80 | 80 | 120 | 70 | 60 | 30% |

For the third pig (Pig 3), the deployable electrode array 442, comprising a reduced number of 3.5 mm needles 14, was used to deliver high density RF lesions for approximately 15 lesion applications, over the outer surface of the stomach ST, over a period of approximately 1 hour. Three treatments were made to the antrum (one in the front region and two in the back region). Table 3 provides ablation procedure data for Pig 3.

TABLE 3
Delivered Ablation Data- 3.5 mm Device - Pig 3

| | | Temp | | Set | | | |
|---|---|---|---|---|---|---|---|
| | Time | Set | Temp | Watt | | Dlvrd | Needle |
| Step | (min) | (° C.) | (° C.) | (W) | Ω | Watt | Density |
| 1 | 2 | 80 | 80 | 120 | 130 | 60 | 50% |
| 2 | 1.5 | 80 | 80 | 120 | 100 | 60 | 50% |
| 3 | 1.5 | 80 | 65 | 120 | 70 | 60 | 50% |
| 4 | 2 | 80 | 80 | 120 | 85 | 60 | 50% |
| 5 | 1.7 | 80 | 78 | 120 | 80 | 60 | 50% |
| 6 | 2 | 80 | 77 | 120 | 76 | 60 | 50% |
| 7 | 2 | 80 | 76 | 120 | 80 | 60 | 50% |
| 8 | 1.3 | 80 | 78 | 120 | 80 | 60 | 50% |
| 9 | 2 | 80 | 82 | 120 | 81 | 60 | 50% |
| 10 | 1.8 | 80 | 81 | 120 | 78 | 60 | 50% |
| 11 | 2 | 80 | 81 | 120 | 73 | 60 | 50% |
| 12 | 2 | 80 | 78 | 120 | 70 | 60 | 50% |
| 13 | 1.8 | 80 | 93 | 120 | 75 | 80 | 50% |
| 14 | 2 | 80 | 78 | 120 | 61 | 60 | 50% |
| 15 | 2 | 80 | 80 | 120 | 80 | 60 | 50% |
| 16 | 2 | 80 | 80 | 120 | 60 | 60 | 50% |

While the application of energy through the needle arrays 442 produced ablation in both the first pig and the second pig, the impact was too severe. The application of lower density energy to the third pig resulted in successful ablation of the stomach ST. Upon recovery from surgery, the appetite of the pig was suppressed, eventually resulting in a 30 percent reduction in weight.

Alternate Applications for Deployable Probe Systems. While the exemplary embodiments have been particularly described for the ablation of a hollow organ HO, such as a stomach ST, the structures and processes are readily adapted for other applications, such as for node sensing and disablement, and/or for applications within a wide variety of other hollow organs, such as within a duodenum, jejunum, ileum, sphincter, or within any desired portion of an upper or lower gastrointestinal tract, or within other hollow organs HO, such as within a uterus. Furthermore, while the exemplary embodiments have been particularly described for the ablation through the interior surface of a hollow organ HO, such as a stomach ST, the structures and processes are readily adapted for ablation through the exterior surface of a hollow organ HO, such as a stomach ST.

As well, while although preferred embodiments are disclosed herein, many variations and/or combinations are possible which remain within the concept, scope, and spirit of the invention. For example, while Applicant has disclosed a deployable apparatus for the application of energy herein, it will be appreciated by those skilled in the art that such the deployable apparatus readily encompasses any device and or process that can be substituted therefore to effect a similar result as is achieved by the deployable apparatus.

Although the ablation systems, mechanisms, and related methods of use are described herein in connection with hollow organ reduction and neural ablation, the systems, mechanisms and techniques can be implemented for a wide variety of applications and uses, or any combination thereof, as desired.

For example, while the exemplary embodiments have been particularly described for the ablation of a hollow organ HO, the structures, processes, and mechanisms are readily adapted for other applications, such as for the acquisition of data and/or the ablation of tissue through electrodes and/or deployable probes as accessed from the outer surface of an organ.

Accordingly, although the invention has been described in detail with reference to a particular preferred embodiment, persons possessing ordinary skill in the art to which this invention pertains will appreciate that various modifications and enhancements may be made without departing from the spirit and scope of the claims that follow.

What is claimed is:

1. A process for providing ablation to a hollow organ, comprising the steps of:
providing an assembly comprising an expandable first balloon having a hollow inner region, a second balloon assembly comprising a hollow expandable region substantially located within the hollow region of the first balloon, at least one deployable active probe which is optionally electrically conductive, and an active element which is optionally an electrical conductor connected to the deployable active probe and extending from the hollow expandable region, and a third balloon comprising a hollow expandable region substantially located within the hollow expandable region of the second balloon;
providing a tube having a first end and a second end, the tube comprising a hollow region defined between the first end and the second end;
inserting the second end of the tube into the hollow organ;
inserting the assembly through the hollow region of the tube and extending from the second end of the tube into the hollow organ;
inflating the first compliant balloon assembly to expand the hollow organ;
inflating the second balloon to substantially contact the first compliant balloon;
inflating the third balloon to deploy the active probe though the first compliant balloon and into contact with the hollow organ.

2. The process of claim 1, wherein the hollow organ is any of a stomach,
a duodenum,
an ileum,
a jejunum,
a sphincter, and
a uterus.

3. The process of claim 1, wherein the second end of the tube is expandable between a first position and a second expanded position.

4. The process of claim 1, wherein the active element is an electrical conductor, further comprising the step of:
measuring the impedance of the deployed probe through the electrical conductor.

5. The process of claim 1, further comprising the step of:
applying energy to the deployed probe through the active element.

6. The process of claim 5, wherein the applied energy is any of RF energy,
radiant energy,
microwave energy, and
laser energy.

7. The process of claim 5, wherein the applied energy is variable.

8. The process of claim 1, wherein the active element is an electrical conductor, further comprising the step of:
measuring the impedance of the deployed probe through the electrical conductor; applying energy to the deployed probe through the electrical conductor;
remeasuring the impedance of the deployed probe through the electrical conductor; and
comparing the measured impedance.

9. The process of claim 1, wherein the assembly further comprises at least one thermal sensor.

10. The process of claim 9, wherein the thermal sensor is any of a thermocouple,
a thermistor, and
an RTD.

11. The process of claim 19, wherein the thermal sensor is in thermal contact with the deployed probe.

12. The process of claim 1, wherein the assembly further comprises a flexible center rod extending into the hollow expandable region of the third balloon.

13. The process of claim 1, further comprising the step of:
filling the inflated first compliant balloon assembly with an electrically conductive solution.

14. The process of claim 13, wherein the electrically conductive solution comprises a saline solution.

15. The process of claim 1, wherein the second balloon assembly further comprises:
means for moving the active probe between an undeployed position and a deployed position.

16. The process of claim 15, wherein the moving means comprises any of a leaf spring,
a coil spring, and
an elastomer spring.

17. The process of claim 15, wherein the moving means further comprises:
a deployment travel limiter.

18. The process of claim 1, wherein the probe is a needle.

19. The process of claim 1, wherein the probe further comprises a hollow region which extends through the probe.

20. The process of claim 1, wherein the probe further comprises an electrically insulative region.

21. The process of claim 1, wherein the probe further comprises a coolant port.

22. The process of claim 1, wherein the second balloon assembly is at least partially electrically conductive.

23. A process for providing ablation to a hollow organ, comprising the steps of:
providing an assembly comprising an expandable first balloon having a hollow inner region, a second balloon assembly comprising a hollow expandable region substantially located within the hollow region of the first balloon, at least one deployable active, electrically conductive probe and a third balloon comprising a hollow expandable region substantially located within the hollow expandable region of the second balloon;
providing a tube having a first end and a second end, the tube comprising a hollow region defined between the first end and the second end;
inserting the second end of the tube into the hollow organ;
inserting the assembly through the hollow region of the tube and extending from the second end of the tube into the hollow organ;
inflating the first compliant balloon assembly to expand the hollow organ;
inflating the second balloon to substantially contact the first compliant balloon;
inflating the third balloon to deploy the electrically conductive probe though the first compliant balloon and into contact with the hollow organ.

24. The process of claim 23, further comprising the step of:
at least partially filling the inflated first compliant balloon with a fluid.

25. The process of claim 23, wherein the fluid is electrically conductive.

26. The process of claim 25, further comprising the step of:
measuring the impedance of the deployed needle through the electrically conductive fluid.

27. The process of claim 25, further comprising the step of:
applying energy to the deployed needle through the electrically conductive fluid.

28. The process of claim 27, wherein the applied energy is any of RF energy,
radiant energy,
microwave energy, and
laser energy.

29. The process of claim 27, wherein the applied energy is variable.

30. The process of claim 25, further comprising the step of:
measuring the impedance of the deployed needle through the electrically conductive fluid;
applying energy to the deployed needle through the electrically conductive fluid;
remeasuring the impedance of the deployed needle through the electrically conductive fluid; and
comparing the measured impedance.

31. The process of claim 24, wherein the fluid comprises a saline solution.

32. The process of claim 31, wherein the fluid further comprises a pharmaceutical solution.

33. The process of claim 23, wherein the hollow organ is any of a stomach,
a duodenum,
an ileum,
a jejunum,
a sphincter, and
a uterus.

34. The process of claim 23, wherein the second end of the tube is expandable between a first position and a second expanded position.

35. The process of claim 23, wherein the assembly further comprises at least one thermal sensor.

36. The process of claim 34, wherein the thermal sensor is any of a thermocouple,
a thermistor, and
an RTD.

37. The process of claim 35, wherein the thermal sensor is in thermal contact with the deployed needle.

38. The process of claim 23, wherein the assembly further comprises a flexible center rod extending into the hollow expandable region of the third balloon.

39. The process of claim 23, wherein the second balloon assembly further comprises:
means for moving the active probe between an undeployed position and a deployed position.

40. The process of claim 38, wherein the moving means comprises any of a leaf spring,
a coil spring,
an elastomer spring, and
a deployment travel limiter.

41. The process of claim 23, wherein the probe is a needle.

42. The process of claim 23, wherein the probe further comprises a hollow region which extends through the probe.

43. The process of claim 23, wherein the probe further comprises an electrically insulative region

44. The process of claim 23, wherein the probe further comprises a coolant port.

45. The process of claim 23, wherein the second balloon assembly is at least partially electrically conductive.

46. An apparatus, comprising:
an expandable first balloon having a hollow inner region;
a second balloon assembly comprising a hollow expandable region substantially located within the hollow region of the first balloon, at least one deployable, active, optionally electrically conductive, needle and an active element, which optionally comprises an electrical conductor, connected to the deployable, active needle and extending from the hollow expandable region; and
a third balloon comprising a hollow expandable region substantially located within the hollow expandable region of the second balloon.

47. The apparatus of claim 46, further comprising:
a flexible center rod extending into the hollow expandable region of the third balloon.

48. The apparatus of claim 46, wherein the second balloon assembly further comprises a thermal sensor.

49. The apparatus of claim 48, wherein the thermal sensor is attached to the deployable active needle.

50. The apparatus of claim 46, wherein the deployable active needle as electrically conductive further comprises an electrically insulative section.

51. The apparatus of 50, wherein the electrically insulative section comprises any of polyimide,
nylon, and
polyester.

52. The apparatus of claim 46, wherein the active needle has a first position, in which the tip is located below the outer surface of the balloon, and a second position, in which the tip is extended from the outer surface of the balloon.

53. The apparatus of claim 51, wherein the second balloon further comprises a hydraulic actuator for movement of the active needle between the first position and the second position.

54. An apparatus, comprising:
an expandable balloon having an outer surface and a hollow inner region having an entrance;
at least one active, optionally electrically conductive probe having a tip located on the balloon, the active probe having a first position, in which the tip is located below the outer surface of the balloon, and a second position, in which the tip is extended from the outer surface of the balloon.

55. The apparatus of claim 54, further comprising:
at least one thermal sensor in thermal contact with the outer surface of the balloon.

56. The apparatus of claim 54, further comprising:
means for applying energy to the active probe.

57. The apparatus of claim 55, wherein the energy is any of RF energy; radiant energy; microwave energy; and laser energy.

58. An apparatus, comprising:
a body having an outer surface;
at least one active electrically conductive probe having a tip located on the body, the active probe having a first position, in which the tip is located below the outer surface of the body, and a second position, in which the tip is extended from the outer surface of the body;
an energy conveying connection to the active probe.

59. The apparatus of claim 58, further comprising:
means for moving the probe from the first position to the second position.

60. The apparatus of claim 58, further comprising:
means for moving the probe from the second position to the first position.

61. The apparatus of claim 58, further comprising:
means for applying energy to the active probe.

62. An apparatus, comprising:
a body having an outer surface, the outer surface having a recessed region;
at least one active, optionally electrically conductive, probe having a tip located on the body within the recessed region;
an orifice extending from the recess region and through the body; and
an energy conveying connection to the active probe.

63. The apparatus of claim 62, further comprising:
means for applying a vacuum to the orifice.

64. The apparatus of claim 62, further comprising:
means for applying energy to the energy conveying connection.

65. An apparatus, comprising:
an expandable balloon having an outer surface and a hollow inner region having an entrance;
at least two electrically conductive probe traces located on the outer surface of the balloon, the electrically conductive probe traces having at least one defined probe region defined there between; and
means for applying energy between the electrically conductive probe traces.

66. The apparatus of claim 65, further comprising:
at least one thermal sensor in thermal contact with the outer surface of the balloon.

67. The apparatus of claim 65, wherein the energy is any of RF energy and microwave energy.

68. The apparatus of claim 65, wherein the expandable balloon further comprises holes extending between the hollow inner region and the outer surface.

69. The apparatus of claim 68, wherein the holes are located in the defined probe region.
